(19) United States
(12) Patent Application Publication
(10) Pub. No.: US 2008/0312271 A1
(43) Pub. Date: Dec. 18, 2008

(54) AZABENZIMIDAZOLYL COMPOUNDS

(75) Inventors: Ivan Efremov, Gales Ferry, CT (US); Bruce N. Rogers, Mystic, CT (US); Allen J. Duplantier, Ledyard, CT (US); Lei Zhang, Waterford, CT (US); Qian Zhang, Levittown, NY (US); Noha S. Maklad, Waterford, CT (US)

Correspondence Address: PFIZER INC;Steve T. Zelson, 150 EAST 42ND STREET, 5TH FLOOR - STOP 49, NEW YORK, NY, 10017-5612, US

(73) Assignee: Pfizer Inc.

(21) Appl. No.: 11/780,569

(22) Filed: Jul. 20, 2007

Related U.S. Application Data
(60) Provisional application No. 60/833,136, filed on Jul. 25, 2006.

(51) Int. Cl. A61K 31/437; C07D 471/04; A61P 25/00; A61P 25/18

(52) U.S. Cl. 514/303; 546/118

(57) ABSTRACT

Compounds and pharmaceutically acceptable salts of the compounds are disclosed, wherein the compounds have the structure of Formula I

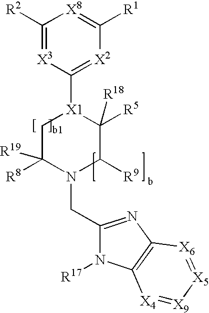

as defined in the specification. Corresponding pharmaceutical compositions, methods of treatment, methods of synthesis, and intermediates are also disclosed.

### CROSS-REFERENCE TO RELATED APPLICATIONS

This application claims priority from U.S. Provisional application Ser. No. 60/833,136 Filed Jul. 7, 2006, which is incorporated by reference in its entirety.

### FIELD OF THE INVENTION

The present invention comprises a novel class of azabenzimidazolyl compounds having the structure of formula (Including tautomers and salts of those compounds) and pharmaceutical compositions comprising a compound of formula I. The present invention also comprises methods of treating a subject by administering a therapeutically effective amount of a compound of formula I to the subject. These compounds are useful for the conditions disclosed herein. The present invention further comprises methods for making the compounds of formula I and corresponding intermediates.

### BACKGROUND OF THE INVENTION

The present invention provides potentiators of glutamate receptors (compounds of formula I), pharmaceutical compositions thereof, and methods of using the same, processes for preparing the same, and intermediates thereof.

Glutamate is an abundant and important neurotransmitter in mammalian CNS that is involved in a variety of normal CNS functions and has been suggested to be involved in CNS disorders. The functions of glutamate as a neurotransmitter are mediated by two families of glutamate receptors on cells in the CNS—the ionotropic glutamate receptor family, which contain integral ion channels, and the metabotropic glutamate receptor family whose members are linked to G-proteins (Ozawa et al., Prog. Neurobiol., 1998, 54, 581-618). The mGlu receptors are part of the Type III G-protein coupled receptor (GPCR) superfamily, which also includes the GABA-B receptors, calcium-sensing receptor, putative pheromone receptors, and taste receptors (Pin et al., Pharmacol Ther., 2003, 98, 325-354).

A key feature in the understanding of many members of the Type III GPCR superfamily that has emerged recently is the recognition of multiple binding sites on these receptors for different classes of pharmacological agents. One class of agents bind to the extracellular endogenous ligand binding site on the receptor (the orthosteric site)—both pharmacological agonists and antagonists that bind to this site have been described for members of the Type III receptor superfamily (Conn and Pin, Ann. Rev. Pharmacol. Toxicol., 1997, 37, 205-237). More recently, for many receptors in the Type III superfamily (including multiple types of mGlu receptors), compounds have been described that bind to regions of the receptor distinct from the orthosteric site (Pin et al., Mol. Pharmacol., 2001, 60, 881-8841. These are termed allosteric ligands, and for many type III receptors the discovery of allosteric ligands has provided pharmacological tools which can be differentiated in chemical structure from orthosteric ligands.

Allosteric compounds may also provide pharmacological distinctions not possible with orthosteric ligands. For example, allosteric compounds may not directly activate a receptor, but rather modulate (by enhancing or reducing) the activity of the endogenous ligand upon its binding to the orthosteric site. In addition, pharmacological distinctions include the potential for pharmacological specificity between related receptors types that share the same endogenous ligand. For example, the structural similarity of the glutamate binding site on closely related members of the mGlu receptor family has resulted in the development of agonist and antagonist compounds that bind to this site which are similar in potency toward multiple receptor within a family. There may be advantages to targeting the development of novel, selective pharmacological agents for these receptors that bind at allosteric sites, since other regions of the receptors show less homology across receptor subtypes than the glutamate binding site.

The metabotropic glutamate (mGlu) receptors include eight subtypes which have been categorized into three groups based on their structural homologies, the second messenger systems to which they are linked, and their pharmacology. The mGlu receptors are found on both CNS neurons and glia, and have been implicated in a variety of CNS functions. Because of the key role of glutamate in CNS function, pharmacological manipulation of this class of glutarnate receptors has been suggested as an avenue to treat a variety of diseases (Conn and Pin, Ann. Rev. Pharmacol. Toxicol., 1997, 37, 205-237; Schoepp and Conn, Trends Pharmacol. Sci., 1993, 14, 13-20).

The present invention relates to the mGluR2 subtype of mGlu receptor, which together with mGluR3 receptors comprise the group II mGlu receptors. mGluR2 receptors have been shown to modulate synaptic transmission at both excitatory glutamate-releasing and inhibitory GABA-releasing neurons (Schoepp, J. Pharmacol Exp. Ther, 2001, 299, 12-20). The pharmacological tools that have been used to probe the functions of mGluR2 receptors are direct agonist and competitive antagonist compounds that have activity at both mGluR2 and mGluR3 receptors. Compounds that bind to allosteric sites of the mGluR2 receptor may allow differentiation from the activities of these orthosteric ligands. Pharmacological manipulations of mGluR2 have been suggested to be useful for a variety of disorders (Marek, Current Opinion in Pharmacology: 2004, 4, 18-22. These include anxiety and related disorders (Tizzano et al., Pharmacol. Biochem., Behav., 2002, 73, 367-374), stress disorders (Eur. J. Pharmacol., 2002, 435, 161-170), depression (Feinberg et al., Pharmacol Biochem, Behav 2002, 73, 467-474), schizophrenia (Klodzinska et al Pharmacol Biochem, Behav., 2002, 73, 327-332; Moghaddam and Adams, Science, 1998, 281, 1349-1352), pain disorders including chronic pain syndromes (Varney and Gereau, Curr. Drug Target CNS Neurol. Disorders, 2002, 1, 283-296), seizure disorders and epilepsy (Moldrich et al. Neuropharmacol., 2001, 41, 8-18), Parkinson's (Bradley et al., J. Neurosci. 2000, 20, 3085-3094), neurodegenerative disorders and brain injury (Bond et al, J Pharmacol Exp. Ther, 2000, 294, 80>$9; Allen et al., J. Pharmacol Exp. Ther., 1999, 290, 112290), and substance abuse (Helton et a, Neuropharmacol., 1998, 36, 1511-1516).

Pin et at., European J, Pharmacology 375 (1999), pp. 277-294, describes the role of mGluR2 agonists and antagonists in regulating the activity of many synapses in the central nervous system, thereby affecting a wide number of physiological and pathological processes.

Johnson et at., J. Med. Chem. 2003, 46, 3189-3192, describes mGluR2 potentiators that have antianxiolytic activity.

All journal articles cited hereinabove are incorporated by reference herein in their entirety.

WO 01/56990 states that mGluR2 receptor potentiators may be effective in the treatment of neurological and psychiatric disorders associated with glutamate dysfunction, including: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache) urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, panic disorder, and obsessive compulsive disorder), mood disorders (including depression, mania, bipolar disorders), trigeminal neuralgia, hearing loss, tinnitus, macular degeneration of the eye, emesis, brain edema, pain (including acute and chronic pain states, severe pain, intractable pain, neuropathic pain, and post-traumatic pain), tardive dyskinesia, sleep disorders (including narcolepsy), attention deficit/hyperactivity disorder, and conduct disorder.

A need still exists for new drug therapies for the treatment of subjects suffering from or susceptible to the above disorders or conditions. In particular, a need still exists for new drugs having one or more improved properties (such as safety profile, efficacy, or physical properties) relative to those currently available.

### SUMMARY OF THE INVENTION

The invention is directed to a class of compounds, including the pharmaceutically acceptable salts of the compounds, having the structure of formula I:

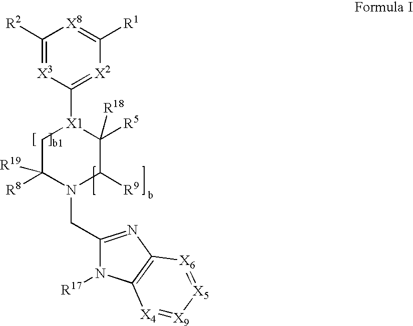

wherein,

X³CR⁶:

X²═CR⁴;

X⁸═CR³;

R¹, R², R³, R⁴ and R⁶ and are each independently selected from the group consisting of hydrogen halogen, —CN, —OR¹⁰¹, alkyl, alkenyl, cycloalkyl, cycloalkenyl, heterocycloalkylaryl, heteroaryl, —C(O)OR¹⁰¹, —C(O)NR¹⁰¹R¹⁰², —NR¹⁰¹R¹⁰², and —NR¹⁰¹S(O)₂R¹⁰³, wherein each of R¹, R², R³, R⁴ and R⁵ alkyl, alkenyl, cycloalkyl, cycloalkenyl, heterocycloalkyl, aryl or heteroaryl is optionally independently substituted with one or more substituents independently selected from the group consisting of halogen, cyano, —R¹⁰¹, —OR¹⁰¹, —NR¹⁰¹R¹⁰², —S(O)_{q}R¹⁰³, —S(O)₂NR¹⁰¹R¹⁰², —NR¹⁰¹S(O)₂R¹⁰³, —OC(O)R¹⁰³, —C(O)OR¹⁰³, —C(O)NR¹⁰¹R¹⁰², —NR¹⁰¹C(O)R¹⁰³, and —C(O)R¹⁰³;

or two substituents bonded to adjacent carbon atoms of the ring containing X², X³ and X⁸, together with the adjacent carbon atoms, form a heterocyclic or carbocyclic ring which is optionally substituted with—one or more R¹⁰, wherein each R¹⁰ is independently selected from the group consisting of hydrogen, cyano, halogen, —C(O)R¹⁰¹, —C(O)NR¹⁰¹R¹⁰², —NR¹⁰¹R¹⁰², —OR¹⁰¹, or —R¹⁰¹;

q is 0, 1 or 2;

each R¹⁰¹ and each R¹⁰² is independently selected from the group consisting of hydrogen, alkyl, alkenyl, alkynyl, cycloalkyl, aryl, heterocycloalkyl and heteroaryl;

wherein each R¹⁰¹ and R¹⁰² alkyl, alkenyl, alkynyl, cycloalkyl, aryl, heterocycloalkyl or heteroaryl is optionally independently substituted with one or more substituents independently selected from the group consisting of halogen, hydroxy, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, haloalkyl, hydroxyalkyl, carboxy, alkoxy, aryloxy, alkoxy carbonyl, aminocarbonyl, alkylaminocarbonyl and dialkylaminocarbonyl;

R¹⁰³ is independently selected from the group consisting of alkyl, alkenyl, cycloalkyl, aryl, heterocycloalkyl and heteroaryl and is optionally substituted with one or more substituents independently selected from the group consisting of halogen hydroxy, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, haloalkyl hydroxyalkyl, carboxy, alkoxy, aryloxy, alkoxycarbonyl, aminocarbonyl, alkylaminocarbonyl and dialkylaminocarbonyl;

X¹═CR⁷;

b=0, 1 or 2;

b1=1 or 2

each of R⁵, R⁸ and R⁹ is independently selected from the group consisting of halogen, cyano, —R⁴⁰¹, —OR⁴⁰¹, —C(O)OR⁴⁰¹ and —NR⁴⁰¹R⁴⁰²;

R⁷ is hydrogen, halogen, hydroxyl, alkyl, alkoxy, cyano or alkyl-CO—;

or R⁵ and R⁷ taken together form a second bond;

R¹⁸ is hydrogen, halogen or alkyl;

R¹⁹ is H or —R⁸ and —R¹⁹ together may form ═O;

wherein R⁴⁰¹ and R⁴⁰² are independently selected from the group consisting of hydrogen, alkyl, alkenyl, cycloalkyl, aryl, heterocycloalkyl and heteroaryl;

wherein each of the R⁴⁰¹ and R⁴⁰² alkyl, alkenyl, cycloalkyl, aryl, heterocycloalkyl and heteroaryl substituents is optionally independently substituted with one or more substituents independently selected from the group consisting of halogen, hydroxy, cyano, nitro, —R⁴¹¹, —C(O)R⁴¹³, —C(O)OR⁴¹³, —C(O)NR⁴¹¹R⁴¹², —OR⁴¹¹, —OC(O)R⁴¹³, —NR⁴¹¹R⁴¹², —NR⁴¹¹C(O)R⁴¹³, NR⁴¹¹C(O)OR⁴¹³, —NR⁴¹¹S(O)₂R⁴¹³, —S(O)ₜR⁴¹³, —S(O)₂NR⁴¹¹R⁴¹²;

t is 0, 1, or 2;

R⁴¹¹ and R⁴¹² are independently selected from the group consisting of hydrogen, alkyl, cycloalkyl, aryl, heterocycloalkyl and heteroaryl;

R⁴¹³ is independently selected from the group consisting of alkyl, cycloalkyl, aryl, heterocycloalkyl and heteroaryl;

wherein the R⁴¹¹, R⁴¹² and R⁴¹³ alkyl, cycloalkyl, aryl, heterocycloalkyl and heteroaryl substituents are each optionally independently substituted with one or more substituents independently selected from the group consisting of halogen, hydroxy, cyano, nitro, alkyl, aryl, heterocycloalkyl, heteroaryl, haloalkyl, hydroxyalkyl, carboxy, alkoxy and alkoxycarbonyl;

or R⁴ and R⁵ together with the atoms connecting R⁴ and R⁵ form a 5-7-membered carbocyclic or heterocyclic ring optionally containing a heteroatom selected from O, N and S;

or b=1 and b1=1, R⁵ and R⁹ together with the atoms connecting R⁵ and R⁹ form a 5-7-membered carbocyclic or heterocyclic ring containing up to two heteroatoms selected from O, N and S, wherein the carbocyclic or heterocyclic ring is optionally substituted with one or more substitutents selected from halogen, cyano, alkyl, cycloalkyl, heterocycloalkyl, aryl, heteroaryl or —C(O)R²⁰, wherein R²⁰ is alkyl, cycloalkyl, heterocycloalkyl, aryl or heteroaryl and R²⁰ is optionally substituted with one or more substituents independently selected from the group consisting of alkyl, alkoxy, aryloxy, cyano, —CO₂-alkyl, and —OC(O)alkyl,

or R⁴ and R⁷ together with the atoms connecting R⁴ and R⁷ form a 5-7-membered carbocyclic or heterocyclic ring, wherein if the ring formed by R⁴ and R⁷ together with the atoms connecting R⁴ and R⁷ is a heterocyclic ring, the heterocyclic ring formed by R⁴ and R⁷ together with the atoms connecting R⁴ and R⁷ contains a heteroatom selected from the group of O, N and S;

or R⁵ and R⁷ together with the atoms connecting R⁵ and R⁷ form a 3-7-membered carbocyclic or heterocyclic ring, wherein if the ring formed by R⁵ and R⁷ together with the atoms connecting R⁵ and R⁷ is a heterocyclic ring, the heterocyclic ring formed by R⁵ and R⁷ together with the atoms connecting R⁵ and R⁷ contains a heteroatom selected from the group of O, N and S;

wherein the carbocyclic or heterocyclic ring formed by R⁴ and R⁷ together with the atoms connecting R⁴ and R⁷, or by R⁶ and R⁷ together with the atoms connecting R⁵ and R⁷ is optionally substituted with one or more substitutents independently selected from halogen, cyano, alkyl, cycloalkyl, heterocycloalkyl, aryl, heteroaryl and C(CO)R²⁰, wherein R²⁰ is alkyl, cycloalkyl, heterocycloalkyl, aryl or heteroaryl and R²⁰ is optionally substituted with one or more alkyl, alkoxy, aryloxy, cyano, CO₂-alkyl, or OC(O)alkyl;

R¹⁷ is selected from the group consisting of alkyl, alkenyl, cycloalkyl, and cycloalkenyl, wherein the R¹⁷ alkyl, alkenyl, cycloalkyl, or cycloalkenyl is optionally substituted with one or more substituents independently selected from the group consisting of halogen, hydroxy, cyano, nitro, —R⁵⁰¹, —OR⁵⁰¹, —NR⁵⁰¹R⁵⁰², —S(O)ᵥR⁵⁰³, —S(O)₂NR⁵⁰¹R⁵⁰², —NR⁵⁰¹S(O)₂R⁵⁰³, —OC(O)R⁵⁰³, —C(O)OR⁵⁰³, —C(O)NR⁵⁰¹R⁵⁰², —NR⁵⁰¹C(O)R⁵⁰³, and —C(O)R⁵⁰³;

v is 0, 1 or 2,

wherein each R⁵⁰¹ and each R⁵⁰² is independently selected from the group consisting of hydrogen, alkyl, alkenyl, cycloalkyl, cycloalkenyl, aryl, heterocycloalkyl and heteroaryl;

X⁴═N or CR¹¹;

X⁹═N or CR¹²;

X⁵═N or CR¹³;

X⁶═N or CR¹⁴;

wherein one or X of X⁴, X⁵, X⁶, and X⁹ are N;

R¹¹, R¹², R¹³ and R¹⁴ are each independently selected from the group consisting of halogen, cyano, —R⁶⁰¹, —C(O)OR⁶⁰¹, —C(O)NR⁶⁰¹R⁶⁰², —OR⁶⁰¹, —NR⁶⁰¹R⁶⁰², and —NR⁶⁰¹C(O)R⁶⁰²;

wherein each R⁶⁰¹ and each R⁶⁰² is independently selected from the group consisting of hydrogen, alkyl, alkenyl, cycloalkyl, cycloalkenyl, aryl, heterocycloalkyl and heteroaryl;

wherein the R⁶⁰¹ and R⁶⁰² alkyl, alkenyl, cycloalkyl, aryl, heterocycloalkyl and heteroaryl substituents are each independently optionally substituted with one or more substituents independently selected from the group consisting of halogen, hydroxy, cyano, nitro, —R⁶¹¹, —C(O)R⁶¹³, —C(O)OR⁶¹³, —C(O)NR⁶¹¹R⁶¹², —OR⁶¹¹, —OC(O)R⁶¹³, —NR⁶¹¹R⁶¹², —NR⁶¹¹C(O)R⁶¹³, —NR⁶¹¹C(O)OR⁶¹³, —NR⁶¹¹S(O)₂R⁶¹³, —S(O)ᵤR⁶¹³, —S(O)₂NR⁶¹¹R⁶¹²;

u is 0, 1 or 2;

each R⁶¹¹ and each R⁶¹² is independently selected from the group consisting of hydrogen, alkyl, cycloalkyl, aryl, heterocycloalkyl and heteroaryl;

each R⁶¹³ is independently selected from the group consisting of alkyl, cycloalkyl, aryl, heterocycloalkyl and heteroaryl;

wherein the R⁶¹¹, R⁶¹² and R⁶¹³ alkyl, cycloalkyl, aryl, heterocycloalkyl and heteroaryl substituents are each independently optionally substituted with one or more substituents independently selected from the group consisting of halogen, hydroxy, cyano, nitro, alkyl, aryl, heterocycloalkyl, heteroaryl, haloalkyl, hydroxyalkyl, carboxy, alkoxy and alkoxycarbonyl;
or R¹¹ and R¹⁷ together with the atoms connecting R¹¹ and R¹⁷ form a 5-8-membered ring containing 1-2 heteroatoms selected from N, O or S wherein the 5-8-membered ring, formed by R¹¹ and R¹⁷ together with the atoms connecting R¹¹ and R¹⁷, is optionally substituted with one or more substitutents independently selected from halogen, cyano, nitro, —R⁶²¹, —C(O)R⁶²³, —OR⁶²¹, —NR⁶²¹R⁶²², —NR⁶²¹C(O)R⁶²³;

R⁶²¹ and R⁶²² are each independently selected from the group consisting of hydrogen, alkyl, cycloalkyl, aryl, heterocycloalkyl and heteroaryl;

R⁶²³ is independently selected from the group consisting of alkyl, cycloalkyl, aryl, heterocycloalkyl and heteroaryl.

In one embodiment of the invention, R¹⁷ is selected from the group consisting of alkyl and cycloalkyl, wherein the R¹⁷ alkyl and cycloalkyl substituents are optionally substituted with one or more substituents independently selected from the group consisting of halogen, cyano, —OR⁵⁰¹, and —NR⁵⁰¹R⁵⁰².

In another embodiment of the invention, at least one of R¹, R², R³, R⁴ and R⁶ is a heterocycloalkyl that contains a nitrogen that is directly bonded to the phenyl ring containing X², X³ and X⁸, wherein the R¹, R², R³, R⁴ or R⁶ heterocycloalkyl is optionally substituted as defined in formula I.

In another embodiment of the invention, at least one of R¹, R², R³, R⁴ and R⁶ is a heteroaryl that contains a nitrogen that is directly bonded to the phenyl ring containing X², X³ and X⁸, wherein the R¹, R², R³, R⁴ or R⁶ heteroaryl is optionally substituted as defined in formula I.

In another embodiment of the invention, R¹⁰¹ is heterocycloalkyl that contains a nitrogen that is directly bonded to the R¹, R², R³, R⁴ or R⁶ alkyl, alkenyl, cycloalkyl, cycloalkenyl, heterocycloalkyl, aryl or heteroaryl, wherein the R¹⁰¹ heterocycloalkyl is optionally substituted as defined in formula I.

In another embodiment of the invention, R¹⁰¹ is heteroaryl that contains a nitrogen that is directly bonded to the R¹, R², R³, R⁴ or R⁶ alkyl, alkenyl, cycloalkyl, cycloalkenyl, heterocycloalkyl, aryl or heteroaryl, wherein the R¹⁰¹ heteroaryl is optionally substituted as defined in formula I.

In another embodiment of the invention, —C(O)R¹⁰³ is —CO-heterocycloalkyl, wherein the heterocycloalkyl contains a nitrogen that is directly bonded to CO, wherein the R¹⁰³ heterocycloalkyl in the COR¹⁰³ is optionally substituted as defined in formula I.

In another embodiment of the invention, —C(O)R¹⁰³ is —CO-heteroaryl, wherein the heteroaryl contains a nitrogen that is directly bonded to CO, wherein the R¹⁰³ heteroaryl in the COR¹⁰³ is optionally substituted as defined in formula I.

In another embodiment of the invention, —SO₂R¹⁰³ is —SO₂heterocycloalkyl, wherein the heterocycloalkyl contains a nitrogen that is directly bonded to SO₂, wherein the R¹⁰³ heterocycloalkyl in the SO₂R¹⁰³ is optionally substituted as defined in formula I.

In another embodiment of the invention, —SO₂R¹⁰³ is —SO₂heteroaryl, wherein the heteroaryl contains a nitrogen that is directly bonded to SO₂, wherein the R¹⁰³ heteroaryl in the SO₂R¹⁰³ is optionally substituted as defined in formula I.

In another embodiment of the invention, R⁷ is hydrogen, fluoro or alkyl.

In another embodiment of the invention, R¹¹, R¹², R¹³ and R¹⁴ are independently selected from the group consisting of hydrogen, halogen, cyano, alkyl, alkoxy, cycloalkyl, aryl, heterocycloalkyl and heteroaryl, wherein the two R¹¹, R¹², R¹³ or R¹⁴ alkyl, cycloalkyl, aryl, heterocycloalkyl and heteroaryl substituents are optionally independently substituted as in the compound of formula I.

Preferably, two of R¹¹, R¹², R¹³ and R¹⁴ are independently selected from the group consisting of hydrogen, cyano and halogen.

In another embodiment of the invention, three of R¹¹, R¹², R¹³ and R¹⁴ are independently selected from the group consisting of hydrogen, halogen, cyano, alkyl, alkoxy, cycloalkyl, aryl, heterocycloalkyl and heteroaryl, wherein the three R¹¹, R¹², R¹³ R¹⁴ alkyl, cycloalkyl, aryl, heterocycloalkyl and heteroaryl substituents are optionally independently substituted as in the compound of formula I. Preferably, the heterocycloalkyl or heteroaryl is substituted with alkoxy.

Preferably, three of R¹¹, R¹², R¹³ and R¹⁴ are independently selected from the group consisting of hydrogen, cyano and halogen.

In another embodiment of the invention, b=1 and b1=0

In another embodiment of the invention, b=1 and b1=1.

In another embodiment of the invention, b and b1 are not both equal to 2.

In another embodiment of the invention, the compound of formula I has the formula II.

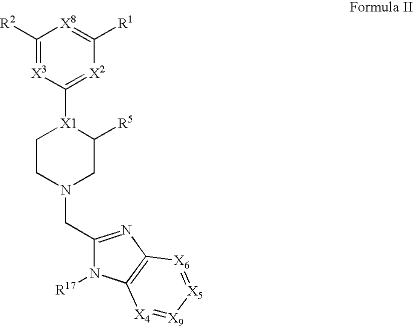

wherein,

R¹, R², R³, R⁴ and R⁸ are each independently selected from the group consisting of hydrogen, halogen, —CN, —OR¹⁰¹, alkyl alkenyl, cycloalkyl, cycloalkenyl, heterocycloalkylaryl, heteroaryl, —C(O)R¹⁰¹, —C(O)OR¹⁰¹, C(O)NR¹⁰¹R¹⁰², —NR¹⁰¹R¹⁰², and NR¹⁰¹S(O)₂R¹⁰³ or, wherein each of R¹, R², R³, R⁴ and R⁶ alkyl, alkenyl, cycloalkyl, cycloalkenyl, heterocycloalkyl, aryl or heteroaryl is optionally independently substituted with one or more substituents independently selected from the group consisting of halogen, cyano, R¹⁰¹, —OR¹⁰¹, —NR¹⁰¹R¹⁰², —S(O)_{q}R¹⁰³, —S(O)₂NR¹⁰¹R¹⁰², —NR¹⁰¹S(O)₂R¹⁰³, —OC(O)R¹⁰³, —C(O)OR¹⁰³, —C(O)NR¹⁰¹R¹⁰², —NR¹⁰¹C(O)R¹⁰³, and —C(O)R¹⁰³;

R⁵ is selected from the group consisting of halogen, —R⁴⁰¹, —OR⁴⁰¹, and —NR⁴⁰¹R⁴⁰²;

R⁷ is hydrogen, halogen, hydroxyl, alkyl, or alkoxy,

or R⁴ and R⁷ together with the atoms connecting R⁴ and R⁷ form a 5-7-membered carbocyclic or heterocyclic ring, wherein if the ring formed by R⁴ and R⁷ together with the atoms connecting R⁴ and R⁷ is a heterocyclic ring, the heterocyclic ring formed by R⁴ and R⁷ together with the atoms connecting R⁴ and R⁷ contains a heteroatom selected from the group of O, N and S;

or R⁵ and R⁷ together with the atoms connecting R⁵ and R⁷ form a 3-7-membered carbocyclic or heterocyclic ring, such as a 5-7-membered carbocyclic or heterocyclic ring, wherein if the ring formed by R⁵ and R⁷ together with the atoms connecting R⁵ and R⁷ is a heterocyclic ring, the heterocyclic ring formed by R⁵ and R⁷ together with the atoms connecting R⁶ and R⁷ contains a heteroatom selected from the group of O, N and S;

wherein the carbocyclic or heterocyclic ring formed by R⁴ and R⁷ together with the atoms connecting R⁴ and R⁷, or by R⁵ and R⁷ together with the atoms connecting R⁵ and R⁷, is optionally substituted with one or more substitutents independently selected from halogen, cyano, alkyl, cycloalkyl, heterocycloalkyl, aryl, heteroaryl and —C(O)R²⁰, wherein R²⁰ is alkyl, cycloalkyl, heterocycloalkyl, aryl or heteroaryl and R²⁰ is optionally substituted with one or more alkyl, alkoxy, aryloxy, cyano, —CO₂-alkyl, or —OC(O)alkyl.

In another embodiment of the compound of formula II, R⁷ is hydrogen or fluoro.

In another embodiment of the compound of formula II, R⁵ is hydrogen, halogen or alkyl optionally substituted with one or more fluorines. In another embodiment of the compound of formula II, R¹⁷ is selected from the group consisting of alkyl and cycloalkyl, wherein the R¹⁷ alkyl and cycloalkyl substituent is optionally substituted as in the compound of formula II.

In another embodiment of the compound of formula II, R¹¹ and R¹⁷ together with the atoms connecting R¹¹ and R¹⁷ form 5-8-membered ring containing one nitrogen atom, wherein R¹¹ and R¹⁷ form a C₂-C₅ alkylene chain optionally substituted with one or more halogen or alkoxy.

In another embodiment of the compound of formula II, two of X⁴, X⁵, X⁶ and X⁹ are N, and two of R¹¹, R¹², R¹³ and R¹⁴ are independently selected from the group consisting of hydrogen, halogen, cyano, alkyl, alkoxy, cycloalkyl, aryl, heterocycloalkyl and heteroaryl, wherein the two R¹¹, R¹², R¹³ or R¹⁴ alkyl, cycloalkyl, aryl, heterocycloalkyl and heteroaryl substituents are optionally independently substituted as in the compound of formula II.

Preferably, two of R¹¹, R¹², R¹³ and R¹⁴ are independently selected from the group consisting of hydrogen, cyano and halogen.

In another embodiment of the compound of formula II, one of X⁴, X⁵, X⁶ and X⁹ is N, and three of R¹¹, R¹², R¹³ and R¹⁴ are independently selected from the group consisting of hydrogen, halogen, cyano, alkyl, alkoxy, cycloalkyl, aryl, heterocycloalkyl and heteroaryl, wherein the three R¹¹, R¹², R¹³ or R¹⁴ alkyl, cycloalkyl, aryl, heterocycloalkyl and heteroaryl substituents are optionally independently substituted as in the compound of formula II.

Preferably, three of R¹¹, R¹², R¹³ and R¹⁴ are independently selected from the group consisting of hydrogen, cyano and halogen.

In another embodiment of the invention, the compound of formula II has the formula III,

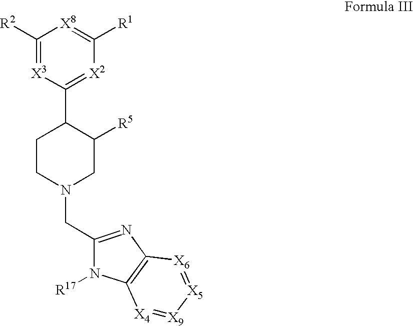

wherein

R¹, R², R³, R⁴ and R⁶ are each independently selected from the group consisting of hydrogen, halogen, —CN, —OR¹⁰¹, alkyl, alkenyl, cycloalkyl, cycloalkenyl, heterocycloalkylaryl, heteroaryl, —C(O)OR¹⁰¹, —C(O)NR¹⁰¹R¹⁰², NR¹⁰¹R¹⁰², and NR¹⁰¹S(O)₂R¹⁰³, or, wherein each of R¹, R², R³, R⁴ and R⁶alkyl, alkenyl, cycloalkyl, cycloalkenyl, heterocycloalkyl, aryl or heteroaryl is optionally independently substituted with one or more substituents independently selected from the group consisting of halogen, cyano, R¹⁰¹, —OR¹⁰¹, —NR¹⁰¹R¹⁰², —S(O)_{q}R¹⁰³, S(O)₂NR¹⁰¹R¹⁰², —NR¹⁰¹S(O)₂R¹⁰³, —OC(O)R¹⁰³, —C(O)OR¹⁰³, —C(O)NR¹⁰¹R¹⁰², —NR¹⁰¹C(O)R¹⁰³, and —C(O)R¹⁰³; and

R⁵ is hydrogen, halogen or alkyl optionally substituted with one or more fluorenes, n one embodiment of Formula III, one of X⁴, X⁵, X⁶ and X⁹ is N, and three of R¹¹, R¹², R¹³ or R¹⁴ are independently selected from the group consisting of hydrogen, halogen, cyano, alkyl, amino, heterocycloalkyl, aryl, and heteroaryl.

In another embodiment of Formula III, one of X⁴, X⁵, X⁶ and X⁹ is N, and three of R¹¹, R¹², R¹³ and R¹⁴ are each independently selected from the group consisting of alkyl, cycloalkyl, heterocycloalkyl, heteroaryl and aryl each optionally independently substituted with one or more substituents independently selected from the group consisting of halogen, alkyl, haloalkyl, alkoxy and alkoxycarbonyl.

In another embodiment of Formula III, two of X⁴,X⁵, X⁶ and X⁹ are N, and two of R¹¹, R¹², R¹³ and R¹⁴ are independently selected from the group consisting of hydrogen, halogen, cyano, alkyl, amino, heterocycloalkyl, aryl, and heteroaryl.

In another embodiment of Formula III, two of X⁴, X⁵, X⁶ and X⁹ are N, and two of R¹¹, R¹², R¹³ and R¹⁴ are each independently selected from the group consisting of alkyl, cycloalkyl, heterocycloalkyl, heteroaryl and aryl each optionally independently substituted with one or more substituents independently selected from the group consisting of halogen, alkyl, haloalkyl, alkoxy and alkoxycarbonyl.

In another embodiment of compounds of formula III, R⁵ is hydrogen.

In another embodiment of compounds of formula III, R⁵ is alkyl or alkyl substituted with one or more fluorines. In another embodiment of compounds of formula III, R⁵ and the aromatic ring containing X², X³ and X⁸ are cis- to each other.

In another embodiment of compounds of formula III, R⁵ is alkyl or cycloalkyl, wherein the R¹⁷ alkyl or cycloalkyl substituent is optionally substituted as in the compound of formula II.

In another embodiment of the compound of formula III, one of X⁴, X⁵, X⁶ and X⁹ is N, and three of R¹¹, R¹², R¹³ and R¹⁴ are independently selected from the group consisting of hydrogen, cyano, halogen, methyl, amino, methoxy, methoxypyridinyl and phenyl.

In another embodiment of the compound of formula III, two of X⁴, X⁵, X⁶ and X⁹ are N, and two of R¹¹, R¹², R¹³, and R¹⁴ are independently selected from the group consisting of hydrogen, cyano, halogen, methyl, amino, methoxy, methoxypyridinyl and phenyl.

In another embodiment of compounds of formula III, R¹⁷ is methyl, cyclopropyl, fluoroethyl, fluoromethyl, methoxyethyl or methoxymethyl.

In another embodiment of the compound of formula III,

R¹⁷ is selected from the group consisting of alkyl and cycloalkyl; wherein R¹⁷ is optionally substituted with one or more substituents independently selected from the group consisting of halogen, alkyl, haloalkyl, alkoxy and alkoxycarbonyl.

In another embodiment of the compound of formula III, R¹⁷ is methyl, cyclopropyl, fluoroethyl, fluoromethyl, methoxyethyl or methoxymethyl;

and either

(a) one of X⁴, X⁵, X⁶ and X⁹ is N, and three of R¹¹, R¹², R¹³, and R¹⁴ are each hydrogen, or

(b) two of X⁴, X⁵, X⁶ and X⁹ are N, and two of R¹¹, R¹², R¹³, and R¹⁴ are each hydrogen.

In another embodiment of the compound of formula III,

R¹⁷ is methyl;

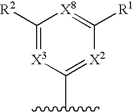

is phenyl optionally substituted with one or more substituents independently selected from the group consisting of halogen, cyano, alkyl, aryl, heterocycloalkyl, heteroaryl, haloalkyl, hydroxyalkyl, carboxy, alkoxy and alkoxycarbonyl; and

X⁵ is N.

In another embodiment of the compound of formula III,

R¹⁷ is methyl;

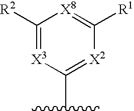

is phenyl optionally substituted with one or more substituents independently selected from the group consisting of halogen, cyano, alkyl, aryl, heterocycloalkyl, heteroaryl, haloalkyl, hydroxyalkyl, carboxy, alkoxy and alkoxycarbonyl; and

X⁴ is N.

In another embodiment of the compound of formula III,

P¹⁷ is methyl:

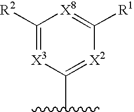

is phenyl optionally substituted with one or more substituents independently selected from the group consisting of halogen, cyano, alkyl, aryl, heterocycloalkyl, heteroaryl, haloalkyl, hydroxyalkyl, carboxy, alkoxy and alkoxycarbonyl; and

X⁹ is N.

In another embodiment of the invention, the compound of formula I has the formula IV,

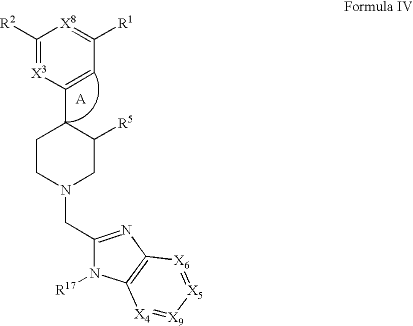

wherein,

X³═CR⁶

X⁸═CR³

R¹, R², R³, and R⁶ are each independently selected from the group consisting of hydrogen, halogen, —CN, —OR¹⁰¹, alkyl, alkenyl, cycloalkyl, cycloalkenyl, heterocycloalkylaryl, heteroaryl —C(O)R¹⁰¹, —C(O)NR¹⁰¹R¹⁰², —NR¹⁰¹R¹⁰², or, wherein each of R¹, R², R³, and R⁶ alkyl, alkenyl, cycloalkyl, cycloalkenyl, heterocycloalkyl, aryl or heteroaryl is optionally independently substituted with one or more substituents independently selected from the group consisting of halogen, cyano, —R¹⁰¹, —OR¹⁰¹, —NR¹⁰¹R¹⁰², —S(O)_{q}R¹⁰³, —S(O)₂NR¹⁰¹R^{102, —NR}¹⁰¹S(O)₂R¹⁰³, —OC(O)OR¹⁰³, —C(O)OR¹⁰³, —C(O)NR¹⁰¹R¹⁰³, —NR¹⁰¹C(O)R¹⁰³, and —C(O)R¹⁰³,

R⁵ is hydrogen, halogen or alkyl;

and wherein ring A is a 5-7-membered carbocyclic or heterocyclic ring, wherein A is optionally substituted with one or more substitutents independently selected from halogen, cyano, alkyl optionally substituted with heterocycloalkyl; cycloalkyl, heterocycloalkyl, aryl, heteroaryl, —C(O)OR²⁰ or —C(O)R²⁰, wherein R²⁰ is alkyl, cycloalkyl, heterocycloalkyl,

aryl or heteroaryl and R²⁰ is optionally substituted with one or more alkyl, alkoxy, aryloxy, cyano, —CO₂-alkyl, or —OC(O)alkyl.

In an exemplary embodiment, the compound of formula IV is a compound of formula IVa.

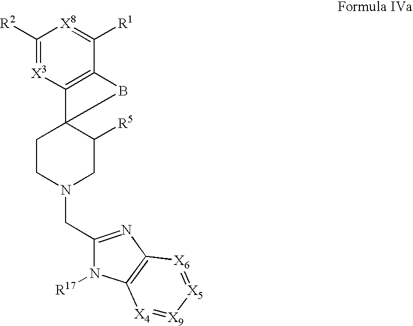

wherein B is a divalent chain selected from the group consisting of ethylene, ethynelene, propylene, butylene, methylenoxy, methylenethioxy, methylenamino, ethylenoxy, ethylenethioxy, and ethylenamino,

wherein the carbons or the N of the methylenamino or ethylenamino divalent chain and the carbons of the ethylene, ethynelene, propylene, butylene, metheylenoxy, ethylenoxy, methylenethioxy, and ethylenethioxy divalent chain are each optionally independently substituted with one or more substitutents independently selected from halogen, cyano, alkyl optionally substituted with heterocycloalkyl, cycloalkyl, heterocycloalkyl, aryl, heteroaryl —C(O)OR²⁰ or —C(O)R²⁰, wherein R²⁰ is alkyl, cycloalkyl, heterocycloalkyl, aryl or heteroaryl and R²⁰ is optionally substituted with one or more alkyl, alkoxy, aryloxy, cyano, —CO₂-alkyl, or —OC(O)alkyl. The foregoing embodiment is intended to include compounds of formula IVa wherein a heteroatom of the divalent chain B is bonded to the carbon of the piperidine ring as well as compounds of formula IVa wherein a heteroatom of the divalent chain B is bonded to the carbon of the ring containing X³ and X⁸.

In one exemplary embodiment, the N of the methylenamino or ethylenamino is optionally substituted with one or more substitutents independently selected from halogen, cyano, alkyl, cycloalkyl, heterocycloalkyl, aryl, heteroaryl or —C(O)R²⁰, wherein R²⁰ is alkyl, cycloalkyl, heterocycloalkyl, aryl or heteroaryl and R²⁰ is optionally substituted with one or more alkyl, alkoxy, aryloxy, cyano, —CO₂-alkyl, or —OC(O)alkyl.

In another embodiment of the invention, the compound of formula I has the formula V,

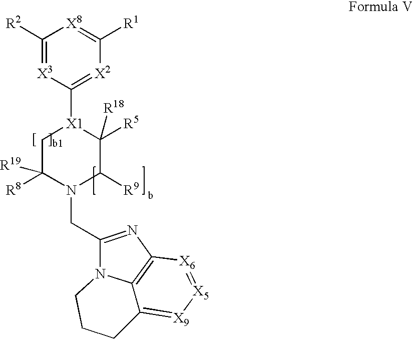

wherein one or two of X⁵, X⁶ and X⁹ are N.

In one embodiment of formula V, X⁶═CR¹⁴ and R¹⁴ is selected from the group consisting of hydrogen and halogen.

In another embodiment of formula V, X⁵═CR¹³ and R¹³ is selected from the group consisting of hydrogen, halogen, cyano, alkyl and amino.

In another embodiment of formula V, X⁹═CR¹² and R¹² is selected from the group consisting of hydrogen, halogen, cyano, alkyl, heterocycloalkyl, and heteroaryl.

In another embodiment of formula V, two of R¹⁴, R¹³ and R¹² are hydrogen.

In an exemplary embodiment of the invention,

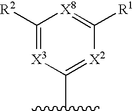

is selected from the group consisting of the following substituents:

4-fluoro-2-methoxyphenyl, 5-fluoro-2-methoxyphenyl, 5-chloro-2-methoxyphenyl, 5-chloro-2-ethoxyphenyl, 5-chloro-2-propoxyphenyl, 5-chloro-2-isobutoxyphenyl, isobutoxyphenyl, butoxyphenyl, 5-Chloro-2-(S)-2-methyl-butoxy)-phenyl, 5-Chloro-2-(R)-2-methyl-butoxy)-phenyl), 2-butoxy-5-chlorophenyl, 5-Chloro-2-(tetrahydro-pyran-2-ylmethoxy phenyl, 5-Chloro-2-(3-methyl-oxetan-3-ylmethoxy)-phenyl, 5-Chloro-2-(tetrahydro-furan-2-ylmethoxy)phenyl, 5-Chloro-2-(tetrahydro-furan-3-ylmethoxy)-phenyl, 5-Chloro-2-(2-methyl-cyclopropylmethoxy)-phenyl, 5-Chloro-2-(2-cyclopropyl-ethoxy)-phenyl, 5-Chloro-2-cyclobutylmethoxy-phenyl, cyclobutylmethoxy-phenyl, 4-fluoro-3-methoxyphenyl, 2-fluoro-6-methoxyphenyl, difluorophenyl, chlorofluorophenyl, chlorophenyl, bromophenyl, dibromophenyl, fluorophenyl, 2-methoxy-4-trifluoromethylphenyl, trifluoromethylphenyl, [dimethylmorpholin-4-yl]methylphenyl, (2-morpholin-4-yl-ethoxy)-phenyl, methylphenyl, dimethylphenyl, 4-chloro-3-trifluoromethylphenyl, methoxyphenyl, dimethoxyphenyl, hydroxyphenyl, phenyl, fluorophenyl, cyclopentylaminocarbonylphenyl, [N-cyclopropylmethyl]propylaminocarbonylphenyl, [methylpyridynyl]aminocarbonylphenyl, fluorochromanyl, ethylphenyl, t-butylphenyl, cyanophenyl, trifluoromethoxyphenyl, isopropoxyphenyl, 2-methoxy-5-trifluoromethylphenyl, 2-fluoro-5-trifluoromethylphenyl, 2-fluoro-4-trifluoromethylphenyl, bis-trifluoromethylphenyl, hydroxyethylphenyl, 4-fluoro-2-methylphenyl, 5-Chloro-2-prop-2-ynyloxy-phenyl, prop-2-ynyloxy-phenyl, naphthalenyl, aminocarbonylnaphthalenyl, (1-phenyl-ethoxy)-phenyl, (Indan-2-yloxy)-phenyl, [(S)-(tetrahydro-furan-3-yl)oxy]-phenyl, (tetrahydro-pyran-4-yloxy-phenyl, ((S)-1 methyl-pyrrolidin-2-ylmethoxy)-phenyl, (2-pyridin-2-yl-ethoxy)-phenyl, ((S)-2-methyl-butoxy)-phenyl, cyclopropyl-ethoxyphenyl, pentoxyphenyl, 3-ethoxypropoxyphenyl, 2-ethoxyethoxyphenyl, 2-isopropoxyethoxyphenyl, 3-dimethylaminopropoxyphenyl, cyclopentylmethoxyphenyl, 2-[2,6Dimethyl-morpholin-4-yl)-ethoxy]-phenyl, (2,6-Dimethyl-morpholin-4-yl)-phenyl, methoxycarbonylphenyl, methylsulfonyamidophenyl, methyl-cyclopropylmethoxyphenyl, propynyloxyphenyl, 5-chloro-2-propynyloxyphenyl, 5-chloro-2-(3-tetrahydrofuranyl)methoxyphenyl, 5-chloro-2-(3-tetrahydropyranyl)methoxyphenyl, 5-chloro-2-(2-tetrahydrofuranyl)methoxyphenyl, 5-chloro-2-(2-tetrahydropyranyl)methoxyphenyl, ethoxyphenyl, N-(5-methyl-1H-pyrazol-3-yl)aminocarbonylphenyl, 3-fluoro-4-trifluoromethyl-phenyl, 2-fluoro-4-trifluoromethoxyphenyl, 2-methyl-4-trifluoromethoxyphenyl, 4-chloro-2-methylphenyl, 4-fluoro-2-methylphenyl, 2-chtoro-4-trifluoromethylphenyl, 2-chloro-4-isopropoxyphenyl, 2-fluoro-4-isopropoxyphenyl, 3-fluoro-4-isopropoxyphenyl, 3-chloro-4-isopropoxyphenyl, 3-chloro-4-ethoxyphenyl, 4-methoxy-2-trifluoromethylphenyl, difluoromethoxyphenyl, 2-fluoro-4-difluoromethoxyphenyl, 2-chloro-4-difluoromethoxyphenyl, trifluorophenyl, tetralinyl, 4-fluoro-2-isopropoxyphenyl, 4-fluoro-3-trifluoromethylphenyl, (2,3-dihydro-1-benzofuran-5-yl), 4-fluoro-2-trifluoromethylphenyl, 4-chloro-2-trifluoromethylphenyl, 2-chloro-4-methylphenyl, 3-chloro-4-trifluoromethoxyphenyl, 2-chloro-4-trifluoromethoxy-phenyl, 2-methoxy-4-trifluoromethoxyphenyl, 2-trifluoromethyl-4-isopropoxyphenyl, 2-fluoro-6-trifluoromethylphenyl, dichlorophenyl, 3-chloro-4-trifluoromethylphenyl, 2-methyl-4-trifluoromethylphenyl, 3-methyl-4-trifluoromethylphenyl, 4-fluoro-2-difluoromethoxyphenyl, 3-methoxy-4-trifluoromethylphenyl.

In the previous substituents, it is understood that, where the relative position of the groups is not specified, any positional isomer is intended to be within the scope of the embodiment. For example, “methoxyphenyl” includes phenyl having a methoxy substituent that may be ortho, meta, or para to the ring containing X1. “Difluorophenyl” includes phenyl having two fluoro substituents that may be ortho, meta, or para to each other, and either of which may be ortho, meta, or para to the ring containing X1. Where the relative position of the groups is specified, the substituent is merely exemplary of any positional isomer having such groups, and such positional isomers are intended to be within the scope of the embodiment.

In another exemplary embodiment of the invention,

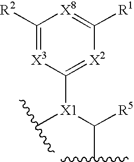

has the structure

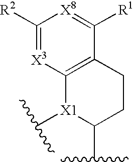

As an example of this embodiment, the aromatic ring containing X³ and X⁸ may be substituted with one or more groups each independently selected from bromo, chloro and methoxy.

Exemplary embodiments of the invention also include embodiments wherein R¹⁷ is selected from the group consisting of the following substituents:

cycloalkyl, such as cyclopropyl;

alkyl, such as methyl or ethyl;

alkyl substituted with halogen, such as fluoroethyl or fluoromethyl; and alkyl substituted with alkoxy, such as methoxyethyl or methoxymethyl.

Exemplary embodiments of the invention also include embodiments wherein each of R¹¹, R¹², R¹³ and R¹⁴ is independently selected from the group consisting of the following substituents;

fluoro, bromo, cyano, chloro alkoxy such as methoxy, aryl such as phenyl, amino, alkylamino, dialkylamino, carboxy, carboxyakyl, carbonylamino, alkylcarbonyl, wherein the alkyl is optionally substituted with one or more alkoxy which is optionally substituted with aryl; cycloalkylcarbonyl; heteroaryl optionally substituted with one or more alkyl or one or more alkoxy, such as methoxypyridinyl; CO-heteroaryl optionally substituted with one or more alkyl or one or more alkoxy; aryl optionally substituted with one or more alkyl or one or more alkoxy or one or more halogen; alkyl such as methyl and alkyl substituted with aryl, hydroxyl, alkoxy, cycloalkyl or halogen.

In one embodiment of the invention, R⁴ and R⁵ together with the atoms connecting R⁴ and R⁵ form a 5-7-membered carbocyclic or heterocyclic ring optionally containing a heteroatom selected from O, N and S in which the carbocyclic or heterocyclic ring and the ring

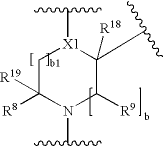

are cis-fused.

In one embodiment of the invention, wherein R⁴ and R⁵ together with the atoms connecting R⁴ and R⁵ form a 5-7-membered carbocyclic or heterocyclic ring optionally containing a heteroatom selected from O, N and 8 in which the carbocyclic or heterocyclic ring and the ring

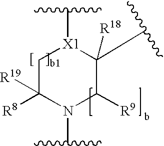

are trans-fused

In another embodiment of the invention, the compound of formula I is an optically active compound of the formula

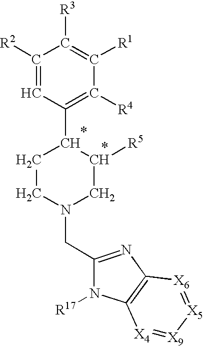

wherein R¹⁷ is as defined in formula I; three of X₆, X₅, X₉ and X₄ are CH and the fourth is N; R¹ and R² are each independently halogen or hydrogen; R³ is halogen, hydrogen, alkyl optionally substituted with halogen, or alkoxy optionally substituted with halogen, R⁴ is halogen, hydrogen, alkyl optionally substituted with halogen, or alkoxy; and R⁵ is alkyl optionally substituted with fluorine, wherein each of the carbons marked with an asterisk independently has the (R) configuration or the (S) configuration, provided that the R⁶ group and the phenyl group substituted with R¹, R², R³ and R⁴ are cis to each other.

In another embodiment of the invention, the compound of formula I is an optically active compound of the formula

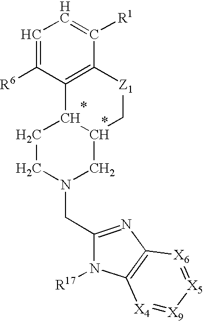

wherein R¹⁷ is as defined in formula I; three of X₆, X₅, X₉ and X₄ are CH and the fourth is N; Z₁ is O or CH₂, R¹ and R² are each independently halogen, hydrogen, or OR¹⁰¹ wherein R¹⁰¹ is alkyl or cycloalkyl, R³ is halogen, hydrogen, alkyl optionally substituted with halogen, or alkoxy optionally substituted with halogen, R⁶ is halogen or hydrogen, wherein each of the carbons marked with an asterisk independently has the (R) configuration or the (S) configuration.

Exemplary compounds according to the invention include the compounds disclosed in Table 8 herein. The compounds of formula I are useful for the treatment or prevention of a variety of neurological and psychiatric, disorders associated with glutamate dysfunction, including: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease. Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, social anxiety disorder, panic disorder, post-traumatic stress disorder and obsessive compulsive disorder), mood disorders (including depression, mania, bipolar disorders), trigeminal neuralgia, hearing loss, tinnitus, macular degeneration of the eye, emesis, brain edema, pain (including acute and chronic pain states, severe pain, intractable pain, neuropathic pain, and posttraumatic pain), tardive dyskinesia, sleep disorders (including narcolepsy), attention deficit/hyperactivity disorder, and conduct disorder. Accordingly, in one embodiment, the invention provides a method for treating or preventing a condition in a mammal, such as a human, selected from the conditions above, comprising administering a compound of formula I to the mammal. The mammal is preferably a mammal in need of such treatment or prevention. As an example, the invention provides a method for treating or preventing a condition selected from migraine, anxiety disorders, schizophrenia, and epilepsy. Exemplary anxiety disorders are generalized anxiety disorder, social anxiety disorder, panic disorder, post-traumatic stress disorder and obsessive compulsive disorder.

In another embodiment, the invention comprises methods of treating or preventing a condition in a mammal, such as a human, by administering a compound having the structure of formula I, wherein the condition is selected from the group consisting of atherosclerotic cardiovascular diseases, cerebrovascular diseases and peripheral arterial diseases, to the mammal. The mammal is preferably a mammal in need of such treatment or prevention. Other conditions that can be treated or prevented in accordance with the present invention include hypertension and angiogenesis.

In another embodiment the present invention provides methods of treating or preventing neurological and psychiatric disorders associated with glutamate dysfunction, comprising: administering to a patient in need thereof an amount of a compound of formula I effective in treating or preventing such disorders. The compound of formula I is optionally used in combination with another active agent. Such an active agent may be, for example, a metabotropic glutamate receptor agonist.

The invention is also directed to a pharmaceutical composition comprising a compound of formula I, and a pharmaceutically acceptable carrier. The composition may be, for example, a composition for treating or preventing a condition selected from the group consisting of acute neurological and psychiatric disorders such as cerebral deficits subsequent to cardiac bypass surgery and grafting, stroke, cerebral ischemia spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug induced Parkinson's disease, muscular spasms and disorders associated with muscular spasticity including tremors, epilepsy, convulsions, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, social anxiety disorder, panic disorder, post-traumatic stress disorder and obsessive compulsive disorder), mood disorders (including depression, mania, bipolar disorders), trigeminal neuralgia, hearing loss, tinnitus, macular degeneration of the eye, emesis, brain edema, pain (including acute and chronic pain states, severe pain, intractable pain, neuropathic pain, and post-traumatic pain), tardive dyskinesia, sleep disorders (including narcolepsy), attention deficit/hyperactivity disorder, and conduct disorder, wherein the composition contains an amount of the compound of formula I that is effective in the treatment or prevention of such conditions. The composition may be, as another example, a composition comprising an mGluR-2 antagonizing amount of the compound of formula I.

The composition may also further comprise another active agent. Such an active agent may be, for example, a metabotropic glutamate receptor agonist.

### DETAILED DESCRIPTION OF THE INVENTION

This detailed description of embodiments is intended only to acquaint others skilled in the art with Applicants' invention, its principles, and its practical application so that others skilled in the art may adapt and apply the invention in its numerous forms, as it may be best suited to the requirements of a particular use. This invention, therefore, is not limited to the embodiments described in this specification, and may be variously modified.

### Abbreviations and Definitions

TABLE A
Abbreviations

| | |
|---|---|
| 1-HOAT | 1-hydroxy-7-azabenzotriazole |
| 1-HOBt | 1-hydroxybenzotriazole hydrate |
| ADP | Adenosine diphosphate (the natural ligand |
| | of P2Y12) |
| AMP | Adenosine monophospate |
| ASA | Acetylsalicylic acid |
| ATP | Adenosine triphosphate |
| Bn | Benzyl group |
| Boc | tert-butoxycarbonyl |
| BOP-Cl | bis(2-oxo-3-oxazolidinyl)phosphinic chloride |
| br | Broad |
| BSA | Bovine serum albumin |
| Cbz | Benzyloxycarbonyl |
| CD₃OD | Deuterated methanol |
| CDCl₃ | Deuterated chloroform |
| CDI | 1,1′-carbonyldiimidazole |
| d | Doublet |
| DBN | 1,5-diazabicyclo[4.3.0]non-5-ene |
| DBU | 1,8-diazabicyclo[5.4.0]undec-7-ene |
| DCC | 1,3-dicyclohexylcarbodiimide |
| DCM | Dichloromethane |
| dd | Doublet of doublets |
| DEPC | diethyl cyanophosphonate |
| DIEA | Diisopropylethylamine |
| DMF | N,N-dimethylformamide |
| DMSO | dimethyl sulphoxide |
| DMC | 2-chloro-1,3-dimethylimidazolinium chloride |
| DPBS | Dulbecco's Phosphate Buffered Saline |
| EBSS | Earle's Balanced Salt Solution |
| EDC | 1-(3-dimethylaminopropyl)-3-ethylcarbodiimide |
| | hydrochloride |
| EDTA | ethylenediaminetetraacetic acid |
| EGTA | ethyleneglycol-bis(β-aminoethyl)-N,N,N′,N′- |
| | tetraacetic Acid |
| ESI | Electrospray Ionization for mass spectrometry |
| Et₃N | Triethylamine |
| EtOAc | ethyl acetate |
| EtOH | Ethanol |
| FBS | Fetal bovine serum |
| Fmoc | Fluorene methyloxycarbonyl |
| HATU | O-(7-azabenzotriazol-1-yl)-N,N,N′,N′-tetramethyluronium |
| | hexafluorophosphate |
| HBTU | O-benzotriazol-1-yl-N,N,N′,N′-tetramethyluronium |
| | hexafluorophosphate |
| HCl | Hydrochloric acid |
| HEK | Human embryonic kidney |
| HEPES | 4-(2-hydroxyethyl)-1-Piperazineethane sulfonic acid |
| HOBT | 1-hydroxybenzotriazole |
| HRMS | High Resolution Mass Spectroscopy (electrospray |
| | ionization positive scan) |
| K₃PO₄ | Potassium phosphate |
| LCMS | Liquid Chromatography - Mass Spectroscopy |
| LRMS | Low Resolution Mass Spectroscopy (electrospray or |
| | thermospray ionization |
| | positive scan) |
| LRMS (ES⁻) | Low Resolution Mass Spectroscopy (electrospray |
| | ionization negative scan) |
| m | Multiplet |
| m/z | Mass spectrum peak |
| MEM | Minimum essential medium |
| MeOH | Methanol |
| MHz | Megahertz |
| MS | Mass spectroscopy |
| NaH | Sodium hydride |
| NMM | N-methylmorpholine |
| NMP | 1-methyl-2-pyrrolidinone |
| NMR | Nuclear Magnetic Resonance |
| PG | Protecting group. Exemplary protecting groups |
| | include Boc, Cbz, Fmoc and |
| | benzyl |
| Pg | Page |
| PPP | Platelet poor plasma |
| PRP | Platelet rich plasma |
| q | Quartet |
| Rpm | Revolutions per minute |
| s | Singlet |
| t | Triplet |
| TFA | trifluoroacetic acid |
| THF | Tetrahydrofuran |
| TLC | Thin layer chromatography |
| Vol. | Volume |
| δ | Chemical shift |
| DEA | Diethylamine |

The term “alkyl” refers to a linear or branched-chain saturated hydrocarbyl substituent (i.e., a substituent obtained from a hydrocarbon by removal of a hydrogen) containing from one to twenty carbon atoms; in one embodiment from one to twelve carbon atoms; in another embodiment, from one to ten carbon atoms, in another embodiment, from one to six carbon atoms; and in another embodiment, from one to four carbon atoms. Examples of such substituents include methyl, ethyl, propyl (including n-propyl and isopropyl), butyl (including n-butyl, isobutyl, sec-butyl and tert-butyl), pentyl, isoamyl, hexyl and the like.

The term “alkenyl” refers to a linear or branched-chain hydrocarbyl substituent containing one or more double bonds and from two to twenty carbon atoms; in another embodiment, from two to twelve carbon atoms; in another embodiment, from two to six carbon atoms; and in another embodiment, from two to four carbon atoms. Examples of alkenyl include ethenyl (also known as vinyl), allyl, propenyl (including 1-propenyl and 2-propenyl) and butenyl (including 1-butenyl, 2-butenyl and 3-butenyl). The term “alkenyl” embraces substituents having “cis” and “trans” orientations, or alternatively, “E” and “Z” orientations.

The term “benzyl” refers to methyl radical substituted with phenyl, i.e.; the following structure:

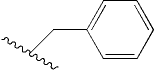

The term “carbocyclic ring” refers to a saturated cyclic, partially saturated cyclic, or aromatic ring containing from 3 to 14 carbon ring atoms (“ring atoms” are the atoms bound together to form the ring). A carbocyclic ring typically contains from 3 to 10 carbon ring atoms. Examples include cyclopropyl, cyclobutyl, cyclopentyl, cyclopentenyl, cyclopentadienyl, cyclohexyl, cyclohexenyl, cyclohexadienyl, and phenyl. A “carbocyclic ring system” alternatively may be 2 or 3 rings fused together, such as naphthalenyl, tetrahydronaphthalenyl (also known as “tetralinyl”), indenyl, isoindenyl, indanyl, bicyclodecanyl, anthracenyl, phenanthrene, benzonaphthenyl (also known as “phenalenyl”), fluorenyl, and decalinyl.

The term “heterocyclic ring” refers to a saturated cyclic, partially saturated cyclic, or aromatic ring containing from 3 to 14 ring atoms (“ring atoms” are the atoms bound together to form the ring), in which at least one of the ring atoms is a heteroatom that is oxygen, nitrogen, or sulfur, with the remaining ring atoms being independently selected from the group consisting of carbon, oxygen, nitrogen, and sulfur.

The term “cycloalkyl” refers to a saturated carbocyclic substituent having three to fourteen carbon atoms. In one embodiment, a cycloalkyl substituent has three to ten carbon atoms. Examples of cycloalkyl include cyclopropyl, cyclobutyl, cyclopentyl and cyclohexyl.

The term “cycloalkyl” also includes substituents that are fused to a C₆-C₁₀ aromatic ring or to a 5-10-membered heteroaromatic ring, wherein a group having such a fused cycloalkyl group as a substituent is bound to a carbon atom of the cycloalkyl group. When such a fused cycloalkyl group is substituted with one or more substituents, the one or more substitutents, unless otherwise specified, are each bound to a carbon atom of the cycloalkyl group. The fused C₆-C₁₀ aromatic ring or to a 5-10-membered heteroaromatic ring may be optionally substituted with halogen, C₃-C₈ alkyl, C₃-C₁₀ cycloalkyl, or ═O.

The term “cycloalkenyl” refers to a partially unsaturated carbocyclic substituent having three to fourteen carbon atoms, typically three to ten carbon atoms. Examples of cycloalkenyl include cyclobutenyl, cyclopentenyl, and cyclohexenyl.

A cycloalkyl or cycloalkenyl may be a single ring, which typically contains from 3 to 6 ring atoms. Examples include cyclopropyl, cyclobutyl, cyclobutyl, cyclopentenyl, cyclopentadienyl, cyclohexyl, cyclohexenyl, cyclohexadienyl, and phenyl. Alternatively, 2 or 3 rings may be fused together, such as bicyclodecanyl and decalinyl.

The term “aryl” refers to an aromatic substituent containing one ring or two or three fused rings. The aryl substituent may have six to eighteen carbon atoms. As an example, the aryl substituent may have six to fourteen carbon atoms. The term “aryl” may refer to substituents such as phenyl, naphthyl and anthracenyl. The term “aryl” also includes substituents such as phenyl, naphthyl and anthracenyl that are fused to a C₄-C₁₀ carbocyclic ring, such as a C₅ or a C₆ carbocyclic ring, or to a 4-10-membered heterocyclic ring, wherein a group having such a fused aryl group as a substituent is bound to an aromatic carbon of the aryl group. When such a fused aryl group is substituted with one more substituents, the one or more substitutents, unless otherwise specified, are each bound to an aromatic carbon of the fused aryl group. The fused C₄-C₁₀ carbocyclic or 4-10-membered heterocyclic ring may be optionally substituted with halogen, C₁-C₆ alkyl, C₃-C₁₀ cycloalkyl, or ═O. Examples of aryl groups include accordingly phenyl, naphthalenyl, tetrahydronaphthalenyl (also known as “tetralinyl”), indenyl, isoindenyl, indanyl, anthracenyl, phenanthrenyl, benzonaphthenyl (also known as “phenalenyl”), and fluorenyl.

In some instances, the number of carbon atoms in a hydrocarbyl substituent (e.g., alkyl, alkenyl, cycloalkyl, cycloalkenyl, aryl, etc.) is indicated by the prefix “Cₓ-C_{y}-,” wherein x is the minimum and y is the maximum number of carbon atoms in the substituent. Thus, for example, “C₃-C₈-alkyl” refers to an alkyl substituent containing from 1 to 6 carbon atoms. Illustrating further, C₃-C₆-cycloalkyl refers to saturated cycloalkyl containing from 3 to 6 carbon ring atoms.

In some instances, the number of atoms in a cyclic substituent containing one or more heteroatoms (e.g., heteroaryl or heterocycloalkyl) is indicated by the prefix “X-Y-membered”, wherein x is the minimum and y is the maximum number of atoms forming the cyclic moiety of the substituent. Thus, for example, 5-8-membered heterocycloalkyl refers to a heterocycloalkyl containing from 5 to 8 atoms, including one or more heteroatoms, in the cyclic moiety of the heterocycloalkyl.

The term “hydrogen” refers to hydrogen substituent, and may be depicted as —H.

The term “hydroxy” refers to —OH. When used in combination with another term(s), the prefix “hydroxy” indicates that the substituent to which the prefix is attached is substituted with one or more hydroxy substituents. Compounds bearing a carbon to which one or more hydroxy substituents include, for example, alcohols, enols and phenol.

The term “hydroxyalkyl” refers to an alkyl that is substituted with at least one hydroxy substituent. Examples of hydroxyalkyl include hydroxymethyl, hydroxyethyl, hydroxypropyl and hydroxybutyl.

The term “nitro” means —NO₂.

The term “cyano” (also referred to as “nitrile”) —CN, which also may be

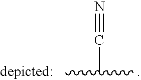

The term “carbonyl” means —C(O)—, which also may be depicted as:

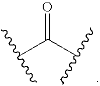

The term “amino” refers to —NH₂.

The term “alkylaimino” refers to an amino group, wherein at least one alkyl chain is bonded to the amino nitrogen in place of a hydrogen atom. Examples of alkylamino substituents include monoalkylamino such as methylamino (exemplified by the formula —NH(CH₃)), which may also be depicted:

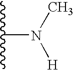

and dialkylamino such as dimethylamino, (exemplified by the formula

—N(CH₃)₂, which may also be depicted:

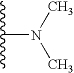

The term “aminocarbonyl” means C(O)—NH₂, which also may be depicted as:

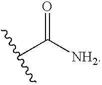

The term “halogen” refers to fluorine (which may be depicted as —F), chlorine (which may be depicted as —Cl), bromine (which may be depicted as —Br), or iodine (which may be depicted as —I). In one embodiment, the halogen is chlorine. In another embodiment, the halogen is a fluorine.

The prefix “halo” indicates that the substituent to which the prefix is attached is substituted with one or more independently selected halogen substituents. For example, haloalkyl refers to an alkyl that is substituted with at least one halogen substituent. Where there are more than one hydrogen replaced with halogens, the halogens may be the identical or different. Examples of haloalkyls include chloromethyl, dichloromethyl, difluorochloromethyl, dichlorofluoromethyl, trichloromethyl, 1-bromoethyl, fluoromethyl, difluoromethyl, trifluoromethyl, 2,2,2-trifluoroethyl, difluoroethyl, pentafluoroethyl, difluoropropyl, dichloropropyl, and heptafluoropropyl. Illustrating further, “haloalkoxy” refers to an alkoxy that is substituted with at least one halogen substituent. Examples of haloalkoxy substituents include chloromethoxy, 1-bromoethoxy, fluoromethoxy difluoromethoxy, trifluoromethoxy (also known as “perfluoromethyloxy”), and 2,2,2-trifluoroethoxy. It should be recognized that if a substituent is substituted by more than one halogen substituent, those halogen substituents may be identical or different (unless otherwise stated).

The prefix “perhalo” indicates that each hydrogen substituent on the substituent to which the prefix is attached is replaced with an independently selected halogen substituent. If all the halogen substituents are identical, the prefix may identify the halogen substituent. Thus, for example, the term “perfluoro” means that every hydrogen substituent on the substituent to which the prefix is attached is replaced with a fluorine substituent. To illustrate, the term “perfpluoroalkyl” refers to an alkyl substituent wherein a fluorine substituent is in the place of each hydrogen substituent. Examples of perfluoroalkyl substituents include trifluoromethyl (—CF₃), perfluorobutyl, perfluoroisopropyl, perfluorododecyl, and perfluorodecyl. To illustrate further, the term “pertluoroalkoxy” refers to an alkoxy substituent wherein each hydrogen substituent is replaced with a fluorine substituent. Examples of perfluoroalkoxy substituents include trifluoromethoxy (—O—CF₃), perfluorobutoxy, perfluoroisopropoxy, perfluorododecoxy, and perfluorodecoxy.

The term “oxo” refers to ═O. The term “oxy” refers to an ether substituent, and may be depicted as —O—.

The term “alkoxy” refers to an alkyl linked to an oxygen, which may also be represented as

—O—R, wherein the R represents the alkyl group. Examples of alkoxy include methoxy, ethoxy, propoxy and butoxy.

The term “alkylthio” means —S-alkyl. For example, “methylthio” is —S—CH₃. Other examples of alkylthio include ethylthio, propylthio, butylthio, and hexylthio.

The term “alkylcarbonyl” means —C(O)-alkyl. For example, “ethylcarbonyl” may be depicted as:

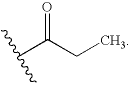

Examples of other alkylcarbonyl include methylcarbonyl, propylcarbonyl, butylcarbonyl, pentylcarbonyl, and hexylcarbonyl.

The term “aminoalkylcarbonyl” means —C(O)-alkyl-NH₂. For example, “aminomethylcarbonyl” may be depicted as

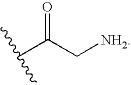

The term “alkoxycarbonyl” means —C(O)—O-alkyl. For example, “ethoxycarbonyl” may be depicted as:

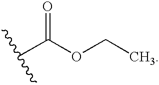

Examples of other alkoxycarbonyl include methoxycarbonyl, ethoxycarbonyl, propoxycarbonyl, butoxycarbonyl, pentoxycarbonyl, and hexyloxycarbonyl. In another embodiment, where the carbon atom of the carbonyl is attached to a carbon atom of a second alkyl, the resulting functional group is an ester.

The terms “thio” and “thia” mean a divalent sulfur atom and such a substituent may be depicted as —S—. For example, a thioether is represented as “alkyl-thio-alkyl” or, alternatively, alkyl-S-alkyl.

The term “thiol” refers to a sulfhydryl substituent, and may be depicted as —SH.

The term “thione” refers to ═S.

The term “sulfonyl” refers to —S(O)₂—, which also may be depicted as:

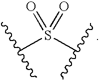

Thus, for example, “alkyl-sulfonyl-alkyl” refers to alkyl-S(O)₂-alkyl. Examples of alkylsulfonyl include methylsulfonyl, ethylsulfonyl, and propylsulfonyl.

The term “aminosulonyl” means —S(O)₂—NH₂, which also may be depicted as:

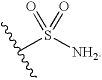

The term “sulfinyl” or “sulfoxido” means —S(O)—, which also may be depicted as;

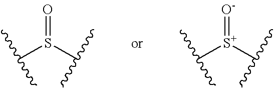

Thus, for example, “alkylsultinylalkyl” or “alkylsulfoxidoalkyl” refers to alkyl-S(O)-alkyl. Exemplary alkylsulfinyl groups include methylsulfinyl, ethylsulfinyl, butylsulfinyl, and hexylsulfinyl.

The term “heterocycloalkyl” refers to a saturated or partially saturated ring structure containing a total of 3 to 14 ring atoms. At least one of the ring atoms is a heteroatom (i.e., oxygen, nitrogen, or sulfur), with the remaining ring atoms being independently selected from the group consisting of carbon, oxygen, nitrogen, and sulfur. A heterocycloalkyl alternatively may comprise 2 or 3 rings fused together, wherein at least one such ring contains a heteroatom as a ring atom (e.g., nitrogen, oxygen, or sulfur). In a group that has a heterocycloalkyl substituent, the ring atom of the heterocycloalkyl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heterocycloalkyl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom.

The term “heterocycloalkyl” also includes substituents that are fused to a C₈-C₁₀ aromatic ring or to a 5-10-membered heteroaromatic ring, wherein a group having such a fused heterocycloalkyl group as a substituent is bound to a heteroatom of the heterocycloalkyl group or to a carbon atom of the heterocycloalkyl group. When such a fused heterocycloalkyl group is substituted with one more substituents, the one or more substitutents, unless otherwise specified, are each bound to a heteroatom of the heterocycloalkyl group or to a carbon atom of the heterocycloalkyl group. The fused C₅-C₁₀ aromatic ring or to a 5-10-membered heteroaromatic ring may be optionally substituted with halogen, C₁-C₆ alkyl, C₃-C₁₀ cycloalkyl, or ═O.

The term “heteroaryl” refers to an aromatic ring structure containing from 5 to 14 ring atoms in which at least one of the ring atoms is a heteroatom (i.e., oxygen, nitrogen, or sulfur), with the remaining ring atoms being independently selected from the group consisting of carbon, oxygen, nitrogen, and sulfur. A heteroaryl may be a single ring or 2 or 3 fused rings. Examples of heteroaryl substituents include 6-membered ring substituents such as pyridyl, pyrazyl, pyrimidinyl, and pyridazinyl, 5-membered ring substituents such as triazolyl, imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl, 6/5-membered fused ring substituents such as benzothiofuranyl, isobenzothiofuranyl, benzisoxazolyl, benzoxazolyl, purinyl, and anthranilyl, and 6/6-membered fused rings such as quinolinyl, isoquinolinyl, cinnolinyl, quinazolinyl, and 1,4-benzoxazinyl. In a group that has a heteroaryl substituent, the ring atom of the heteroaryl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heteroaryl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom. The term “heteroaryl” also includes pyridyl N-oxides and groups containing a pyridine N-oxide ring,

Examples of single-ring heteroaryls include furanyl, dihydrofuranyl, tetrahydrofuranyl, thiophenyl (also known as “thiofuranyl”), dihydrothiophenyl, tetrahydrothiophenyl, pyrrolyl, isopyrrolyl, pyrrolinyl, pyrrolidinyl imidazolyl isoimidazolyl, imidazolinyl; imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiaediazolyl, oxathiazolyl oxadiazolyl (including oxadiazolyl, 1,2,4-oxadiazolyl (also known as “azoximyl”), 1,2,5-oxadiazolyl (also known as “furazanyl”), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), oxathiazolyl, oxathiolyl, oxathiolanyl, pyranyl (including 1,2-pyranyl or 1,4-pyranyl), dihydropyranyl, pyridinyl (also known as “azinyl”), piperidinyl, diazinyl (including pyridazinyl (also known as “1,2-diazinyl”), pyrimidinyl (also known as “1,3-diazinyl” or “pyrimidyl”), or pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1,2,3-triazinyl”)), oxazinyl (including 1,2,3-oxazinyl, 1,3,2-oxazinyl, 1,3,6-oxazinyl (also known as “pentoxazolyl”), 1,2,6-oxazinyl, or 1,4-oxazinyl), isoxazinyl (including o-isoxazinyl or p-isoxazinyl), oxazolidinyl, isoxazolidinyl, oxathiazinyl (including 1,2,5-oxathiazinyl or 1,2,6-oxathiazinyl), oxadiazinyl (including 1,4,2-oxadiazinyl or 1,3,5,2-oxadiazinyl), morpholinyl, azepinyl, oxepinyl, thiepinyl, and diazepinyl.

Examples of 2-fused-ring heteroaryls include, indolizinyl, pyrindinyl, pyranopyrrolyl, 4H-quinolizinyl, purinyl, naphthyridinyl, pyridopyridinyl (including pyrido[3,4-b]-pyridinyl, pyrido[3,2-b]-pyrindinyl, or pyrido[4,3-b]-pyridinyl), and pteridinyl, indolyl, isoindolyl, indoleninyl, isoindazolyl, benzazinyl, phthalazinyl, quinoxalinyl, quinazolinyl, benzodiazinyl, benzopyranyl, benzothiopyranyl, benzoxazolyl, indoxazinyl, anthranilyl, benzodioxolyl, benzodioxanyl, benzoxadiazolyl, benzofuranyl, isobenzofuranyl, benzothienyl, isobenzothienyl, benzothiazolyl, benzothiadiazolyl, benzimidazolyl, benzotriazolyl, benzoxazinyl, benzisoxazinyl, and tetrahydroisoquinolinyl.

Examples of 3-fused-ring heteroaryls or heterocycloalkyls include 5,6-dihydro-4H-imidazo[4,5,1-ij]quinoline, 4,5-dihydroimidazo[4,5,1-hi]indole, 4,5,6,7-tetrahydroimidazo[4,5,1-jk][1]benzazepine, and dibenzofuranyl.

Other examples of fused-ring heteroaryls include benzo-fused heteroaryls such as indolyl, isoindolyl (also known as “isobenzazolyl” or “pseudoisoindolyl”), indoleninyl (also known as “pseudoindolyl”), isoindazolyl (also known as ‘benzpyrazolyl’), benzazinyl (including quinolinyl (also known as “1-benzazinyl”) or isoquinolinyl (also known as “2-benzazinyl”)), phthalazinyl, quinoxalinyl, quinazolinyl, benzodiazinyl (including cinnolinyl (also known as “1,2-benzodiazinyl”) or quinazolinyl (also known as “1,3-benzodiazinyl”)), benzopyranyl (including “chromanyl” or “isochromanyl”), benzothiopyranyl (also known as “thiochromanyl”), benzoxazolyl, indoxazinyl (also known as “benzisoxazolyl”), anthranilyl, benzodioxolyl, benzodioxanyl, benzoxadiazolyl, benzofuranyl (also known as “courmaronyl”), isobenzofuranyl, benzothienyl (also known as “benzothiophenyl,” “thionaphthenyl,” or “benzothiofuranyl”), isobenzothienyl (also known as “isobenzothiophenyl,” “isothionaphthenyl,” or “isobenzothiofuranyl”), benzothiazolyl, benzothiadiazolyl, benzimidazolyl, benzotriazolyl, benzoxazinyl (including 1,3,2-benzoxazinyl, 1,4,2-benzoxazinyl, 2,3,1-benzoxazinyl, or 3,1,4-benzoxazinyl), benzisoxazinyl (including 1,2-benzisoxazinyl or 1,4-benzisoxazinyl), tetrahydroisoquinolinyl, carbazolyl, xanthenyl, and acridinyl.

The term “heteroaryl” also includes substituents such as pyridyl and quinolinyl that are fused to a C₄-C₁₀ carbocyclic ring, such as a C₅ or a C₈ carbocyclic ring, or to a 4-10-membered heterocyclic ring, wherein a group having such a fused aryl group as a substituent is bound to an aromatic carbon of the heteroaryl group or to a heteroatom of the heteroaryl group. When such a fused heteroaryl group is substituted with one more substituents, the one or more substitutents, unless otherwise specified, are each bound to an aromatic carbon of the heteroaryl group or to a heteroatom of the heteroaryl group. The fused C₄-C₁₀ carbocyclic or 4-10-membered heterocyclic ring may be optionally substituted with halogen, C₁-C₆ alkyl, C₃-C₁₀ cycloalkyl, or ═O.

The term “ethylene” refers to the group —CH₂—CH₂—.

The term “ethyneilene” refers to the group —CH═CH—.

The term “propylene” refers to the group —CH₂—CH₂—CH₂—.

The term “butylene” refers to the group —CH₂—CH₂—CH₂—CH₂—.

The term “methylenoxy” refers to the group —CH₂—O—.

The term “ethylenethioxy” refers to the group —CH₂—S—.

The term “methylenamino” refers to the group —CH₂—N(H)—.

The term “ethylenoxy” refers to the group —CH₂—CH₂—O—.

The term “ethylenethioxy” refers to the group —CH₂—CH₂—S—.

The term “ethylenamino” refers to the group —CH₂—CH₂—N(H)—.

A substituent is “substitutable” if it comprises at least one carbon, sulfur, oxygen or nitrogen atom that is bonded to one or more hydrogen atoms. Thus, for example, hydrogen, halogen, and cyano do not fall within this definition.

If a substituent is described as being “substituted,” a non-hydrogen substituent is in the place of a hydrogen substituent on a carbon, oxygen, sulfur or nitrogen of the substituent. Thus, for example, a substituted alkyl substituent is an alkyl substituent wherein at least one non-hydrogen substituent is in the place of a hydrogen substituent on the alkyl substituent. To illustrate, monofluoroalkyl is alkyl substituted with a fluoro substituent, and difluoroalkyl is alkyl substituted with two fluoro substituents. It should be recognized that if there is more than one substitution on a substituent, each non-hydrogen substituent may be identical or different (unless otherwise stated).

If a substituent is described as being “optionally substituted,” the substituent may be either (1) not substituted, or (2) substituted. If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is described as being optionally substituted with one or more of a list of substituents, one or more of the hydrogens on the nitrogen (to the extent there are any) may each be replaced with an independently selected optional substituent. One exemplary substituent may be depicted as —NR′R,″ wherein R′ and R″ together with the nitrogen atom to which they are attached, may form a heterocyclic ring. The heterocyclic ring formed from R′ and R″ together with the nitrogen atom to which they are attached may be partially or fully saturated. In one embodiment, the heterocyclic ring consists of 3 to 7 atoms. In another embodiment, the heterocyclic ring is selected from the group consisting of pyrrolyl, imidazolyl, pyrazolyl, triazolyl, tetrazolyl, isoxazolyl, pyridyl and thiazolyl.

This specification uses the terms “substituent,” “radical,” and “group” interchangeably.

If a group of substituents are collectively described as being optionally substituted by one or more of a list of substituents, the group may include: (1) unsubstitutable substituents, (2) substitutable substituents that are not substituted by the optional substituents, and/or (3) substitutable substituents that are substituted by one or more of the optional substituents.

If a substituent is described as being optionally substituted with up to a particular number of non-hydrogen substituents, that substituent may be either (1) not substituted; or (2) substituted by up to that particular number of non-hydrogen substituents or by up to the maximum number of substitutable positions on the substituent, whichever is less. Thus, for example, if a substituent is described as a heteroaryl optionally substituted with up to 3 non-hydrogen substituents, then any heteroaryl with less than 3 substitutable positions would be optionally substituted by up to only as many non-hydrogen substituents as the heteroaryl has substitutable positions. To illustrate, tetrazolyl (which has only one substitutable position) would be optionally substituted with up to one non-hydrogen substituent. To illustrate further, if an amino nitrogen is described as being optionally substituted with up to 2 non-hydrogen substituents, then the nitrogen will be optionally substituted with up to 2 non-hydrogen substituents if the amino nitrogen is a primary nitrogen, whereas the amino nitrogen will be optionally substituted with up to only 1 non-hydrogen substituent if the amino nitrogen is a secondary nitrogen.

A prefix attached to a multi-moiety substituent only applies to the first moiety. To illustrate, the term “alkylcycloalkyl” contains two moieties: alkyl and cycloalkyl. Thus, a C₁-C₆-prefix on C₃-C₆-alkylcycloalkyl means that the alkyl moiety of the alkylcycloalkyl contains from 1 to 6 carbon atoms; the C₁-C₆-prefix does not describe the cycloalkyl moiety. To illustrate further, the prefix “halo” on haloalkoxyalkyl indicates that only the alkoxy moiety of the alkoxyalkyl substituent is substituted with one or more halogen substituents. If the halogen substitution may only occur on the alkyl moiety, the substituent would be described as alkoxyhaloalkyl. If the halogen substitution may occur on both the alkyl moiety and the alkoxy moeity, the substituent would be described as “haloalkoxyhaloalkyl.”

When a substituent is comprised of multiple moieties, unless otherwise indicated, it is the intention for the final moiety to serve as the point of attachment to the remainder of the molecule. For example, in a substituent A-B-C, moiety C is attached to the remainder of the molecule. In a substituent A-B-C-D, moiety D is attached to the remainder of the molecule. Similarly, in a substituent aminocarbonylmethyl, the methyl moiety is attached to the remainder of the molecule, where the substituent may also be depicted as

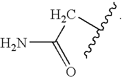

In a substituent trifluoromethylaminocarbonyl, the carbonyl moiety is attached to the remainder of the molecule, where the substituent may also be depicted as

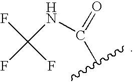

If substituents are described as being “independently selected” from a group, each substituent is selected independent of the other. Each substituent therefore may be identical to or different from the other substituent(s).

### Isomers

When an asymmetrc center is present in a compound of formulae I through V, hereinafter referred to as the compound of the invention, the compound may exist in the form of optical isomers (enantiomers). In one embodiment, the present invention comprises enantiomers and mixtures, including racemic mixtures of the compounds of formulae E through V. In another embodiment, for compounds of formulae I through V that contain more than one asymmetric center, the present invention comprises diastereoameric forms (individual diastereomers and mixtures thereof) of compounds. When a compound of formulae I through V contains an alkenyl group or moiety, geometric isomers may arise.

### Tautomeric Forms

The present invention comprises the tautomeric forms of compounds of formulae I through V. Where structural isomers are interconvertible via a low energy barrier, tautomeric isomerism (“tauftomerisim”) can occur. This can take the form of proton tautomerism in compounds of formula I containing, for example, an imino, keto, or oxime group, or so-called valence tautomerism in compounds which contain an aromatic moiety. It follows that a single compound may exhibit more than one type of isomerism. The various ratios of the tautomers in solid and liquid form is dependent on the various substituents on the molecule as well as the particular crystallization technique used to isolate a compound.

### Salts

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable saft” refers to a salt prepared by combining a compound of formulae I-V with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention which are generally prepared by reacting the free base with a suitable organic or inorganic acid.

Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclylic, carboxylic, and sulfonic classes of organic acids.

Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate.

Furthermore, where the compounds of the invention carry an acidic moiety, suitable pharmaceutically acceptable salts thereof may include alkali metal salts, e.g., sodium or potassium salts, alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

Organic salts may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₈) halides (e.g. methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), arylalkyl halides (e.g., benzyl and phenethyl bromides), and others.

In one embodiment, hemisalts of acids and bases may also be formed, for example, hemisulphate and hemicalcium salts.

The compounds of the invention may exist in both unsolvated and solvated forms. A “solvate” as used herein is a nonaqueous solution or dispersoid in which there is a noncovalent or easily dispersible combination between solvent and solute, or dispersion means and disperse phase.

### Prodrugs

Also within the scope of the present invention are so-called “prodrugs” of the compound of the invention. Thus, certain derivatives of the compound of the invention which may have little or no pharmacological activity themselves can, when administered into or onto the body, be converted into the compound of the invention having the desired activity, for example, by hydrolytic cleavage. Such derivatives are referred to as “prodrugs.” Further information on the use of prodrugs may be found in “Pro-drugs as Novel Delivery Systems, Vol. 14, ACS Symposium Series (T Higuchi and W Stella) and “Bioreversible Carriers in Drug Design,” Pergamon Press, 1987 (ed. E B Roche. American Pharmaceutical Association). Prodrugs in accordance with the invention can, for example, be produced by replacing appropriate functionalities present in the compounds of any of formulae I through V with certain moieties known to those skilled in the art as “pro-moieties” as described, for example, in “Design of Prodrugs” by H Bundgaard (Elsevier, 1985).

### Isotopes

The present invention also includes isotopically labelled compounds, which are identical to those recited in formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁶F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated. i.e., ³H and carbon-14, i.e. ¹⁴C. isotopes are particularly preferred for their ease of preparation and detectability. Further, substitution with heavier isotopes such as deuterium, i.e., ²H, can afford certain therapeutic advantages resulting from greater metabolic, stability, for example increased in vivo half-life or reduced dosage requirements and, hence, may be preferred in some circumstances, isotopically labelled compounds of formula I of this invention and prodrugs thereof can generally be prepared by carrying out the procedures disclosed in the Schemes and/or in the Examples and Preparations below, by substituting a readily available isotopically labelled reagent for a non-isotopically labelled reagent.

### Administration and Dosing

Typically, a compound of the invention is administered in an amount effective to treat or prevent a condition as described herein. The compounds of the invention are administered by any suitable route in the form of a pharmaceutical composition adapted to such a route, and in a dose effective for the treatment or prevention intended. Therapeutically effective doses of the compounds required to treat or prevent the progress of the medical condition are readily ascertained by one of ordinary skill in the art using preclinical and clinical approaches familiar to the medicinal arts

The compounds of the invention may be administered orally. Oral Administration may involve swallowing, so that the compound enters the gastrointestinal tract, or buccal or sublingual administration may be employed by which the compound enters the blood stream directly from the mouth.

In another embodiment, the compounds of the invention may also be administered directly into the blood stream, into muscle, or into an internal organ Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the compounds and/or compositions containing the compounds is based on a variety of factors, including the type, age, weight, sex and medical condition of the patient, the severity of the condition; the route of administration and the activity of the particular compound employed. Thus the dosage regimen may vary widely, Dosage levels of the order from about 0.01 mg to about 100 mg per kilogram of body weight per day are useful in the treatment or prevention of the above-indicated conditions. In one embodiment, the total daily dose of a compound of the invention (administered in single or divided doses) is typically from about 0.01 to about 100 mg/kg. In another embodiment, total daily dose of the compound of the invention is from about 0.1 to about 50 mg/kg, and in another embodiment, from about 0.5 to about 30 mg/kg (i.e., mg compound of the invention per kg body weight). In one embodiment, dosing is from 0.01 to 10 mg/kg/day. In another embodiment, dosing is from 0.1 to 1.0 mg/kg/day. Dosage unit compositions may contain such amounts or submultiples thereof to make up the daily dose. In many instances, the administration of the compound will be repeated a plurality of times in a day (typically no greater than 4 times). Multiple doses per day typically may be used to increase the total daily dose, if desired.

For oral administration, the compositions may be provided in the form of tablets containing 0.01, 0.05, 0.1, 0.5, 1.0, 2.5, 5.0, 10.0, 15.0, 25.0, 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.1 to about 10 mg/kg/minute during a constant rate infusion.

Suitable subjects according to the present invention include mammalian subjects. Mammals according to the present invention include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

### Use in the Preparation of a Medicament

In another embodiment, the invention comprises the use of one or more compounds of the invention for the preparation of a medicament for the treatment or prevention of the conditions recited herein.

### Pharmaceutical Compositions

For the treatment or prevention of the conditions referred to above, the compound of the invention can be administered as compound per se. Alternatively, pharmaceutically acceptable salts are suitable for medical applications because of their greater aqueous solubility relative to the parent compound.

In another embodiment, the present invention comprises pharmaceutical compositions. Such pharmaceutical compositions comprise a compound of the invention presented with a pharmaceutically-acceptable carrier. The carrier can be a solid, a liquid, or both, and may be formulated with the compound as a unit-dose composition, for example, a tablet, which can contain from 0.05% to 95% by weight of the active compounds. A compound of the invention may be coupled with suitable polymers as targetable drug carriers. Other pharmacologically active substances can also be present.

The compounds of the present invention may be administered by any suitable route, preferably in the form of a pharmaceutical composition adapted to such a route, and in a dose effective for the treatment or prevention intended. The active compounds and compositions, for example, may be administered orally, rectally, parenterally, or topically.

Oral administration of a solid dose form may be, for example, presented in discrete units, such as hard or soft capsules, pills, cachets, lozenges, or tablets, each containing a predetermined amount of at least one compound of the present invention. In another embodiment, the oral administration may be in a powder or granule form. In another embodiment, the oral dose form is sub-lingual, such as, for example, a lozenge. In such solid dosage forms, the compounds of formulae I through V are ordinarily combined with one or more adjuvants. Such capsules or tablets may contain a controlled-release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In another embodiment, the present invention comprises a parenteral dose form “Parenteral administration” includes, for example, subcutaneous injections, intravenous injections, intraperitoneally, intramuscular injections, intrasternal injections, and infusion. Injectable preparations (e.g., sterile injectable aqueous or oleaginous suspensions) may be formulated according to the known art using suitable dispersing, wetting agents, and/or suspending agents.

In another embodiment, the present invention comprises a topical dose form “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Formulations suitable for topical administration to the eye include, for example, eye drops wherein the compound of this invention is dissolved or suspended in suitable carrier. A typical formulation suitable for ocular or aural administration may be in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the present invention comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E. Remington's Pharmaceutical Sciences, Mack Publishing Co. Easton, Pa., 1975, Liberman, et al., Eds., Pharmaceutical Dosage Forms, Marcel Decker, New York, N.Y., 1980, and Kibbe, et al., Eds., Handbook of Pharmaceutical Excipients (3^{rd} Ed.), American Pharmaceutical Association, Washington, 1999.

### Co-administration

The compounds of the present invention can be used, alone or in combination with other therapeutic agents, in the treatment or prevention of various conditions or disease states. The compound(s) of the present invention and other therapeutic agent(s) may be may be administered simultaneously (either in the same dosage form or in separate dosage forms) or sequentially. An exemplary therapeutic agent may be, for example, a metabotropic glutamate receptor agonist.

The administration of two or more compounds “in combination” means that the two compounds are administered closely enough in time that the presence of one Walters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

### Kits

The present invention further comprises kits that are suitable for use in performing the methods of treatment or prevention described above. In one embodiment, the kit contains a first dosage form comprising one or more of the compounds of the present invention and a container for the dosage, in quantities sufficient to carry out the methods of the present invention.

In another embodiment, the kit of the present invention comprises one or more compounds of the invention

### Intermediates

In another embodiment, the invention relates to the novel intermediates useful for preparing the compounds of the invention

### General Synthetic Schemes

The compounds of the formula I may be prepared by the methods described below, together with synthetic methods known in the art of organic chemistry, or modifications and derivatisation that are familiar to those of ordinary skill in the art. The starting materials used herein are commercially available or may be prepared by routine methods known in the art (such as those methods disclosed in standard reference books such as the COMPENDIULM OF ORGANIC SYNTHETIC METHODS, Vol. I-VI (published by Wiley-Interscience)). Preferred methods include, but are not limited to, those described below.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T W Greene and P. G. M. Wuts, Protective Groups in Organic Chemistry, John Wiley & Sons, 1991, and T. W. Greene and P.C. M. Wuts, Protective Groups in Organic Chemistry. John Wiley & Sons. 1999, which are hereby incorporated by reference.

Compounds of formula I, or their pharmaceutically acceptable salts, can be prepared according to the reaction Schemes discussed hereinbelow. Unless otherwise indicated, the substituents in the Schemes are defined as above. Isolation and purification of the products is accomplished by standard procedures, which are known to a chemist of ordinary skill.

The following schemes are exemplary of the processes for making compounds of formula I. In the schemes below, the numerals used, including numerals from (I) to (V), are used for convenience to designate the formulae in the schemes. The use of numerals from (I) to (V) in the schemes below is not intended to imply that the compounds designated by such numerals correspond to the compounds of formulae I V that are disclosed hereinabove and that are recited in the appended claims.

Scheme I illustrates a method for the preparation of compounds of formula I, where R¹ to R¹⁹ and X¹ to X⁸ are defined as above.

Referring to scheme I, a compound of formula (I) can be synthesized by treating secondary amine of formula (II) with the aldehydes of formula (II) in the presence of suitable reducing agents such as NaBH(OAc)₃, or Na(CN)BH₃, in solvents such as methylene chloride, dichloroethane, DMF or THF, at about room temperature. Other suitable conditions for this transformation include treatment of the amine of formula (II) with aldehydes of formula (III) in solvents such as methanol or ethanol at room temperature, followed by treatment with reducing agents such as NaBH₄ or NaCNBH₃₂, Which also produce the desired compounds of formula (I).

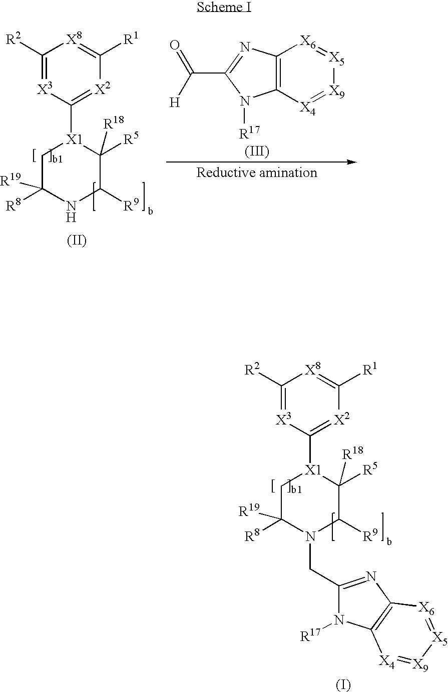

Aldehydes of formula (III) are either commercially available or can be prepared, but not limited to, by general procedures illustrated by scheme II, wherein R¹⁷, X⁴, X⁵, X⁶, and X⁹ are defined as above. Referring to scheme II below, haloheteroaryls (IV) can be treated with primary amines of formula (V) in the presence of a suitable base such as potassium carbonate and the like, in a suitable solvent such as dichloromethane at a reaction temperature ranging from room temperature to 100° C. to give compounds of formula (VI). Hydrogenation of the nitro group using well-precedented conditions such as Pd/C under hydrogen or Fe/EtOH/CaCl₂ can yield diamine of formula (VIII). The imidazole ring can be formed by treating diamines (XIV) with acetamidates of formula (XI), in the presence of acetic acid, in a suitable solvent such as MeOH. The acetal of compounds (XVII) can be removed with acids such as HCl to give the desired aldehydes of formula (V). Alternatively, diamines (VII) can be condensed with glycolic acid under strong acidic conditions, such as aqueous hydrochloric acid, at elevated temperature such as reflux. The resultant alcohols of formula (IX) can then be oxidized using a suitable oxidation reagent, such as MnO₂ in a suitable solvent such as methylene chloride, to yield the desired aldehydes of formula (V). In addition, diamines (VII) can cyclize with triethylorthoacetate in a suitable solvent such as ethanol at elevated temperature with or without microwave heating to produce imidazoles of formula (VII), which can be subsequently oxidized to the desired aldehydes of formula (V) using selenium dioxide. Other known literature procedures on synthesis of methylbenzimidazole aldehydes or small variations of the synthesis described above can also be used.

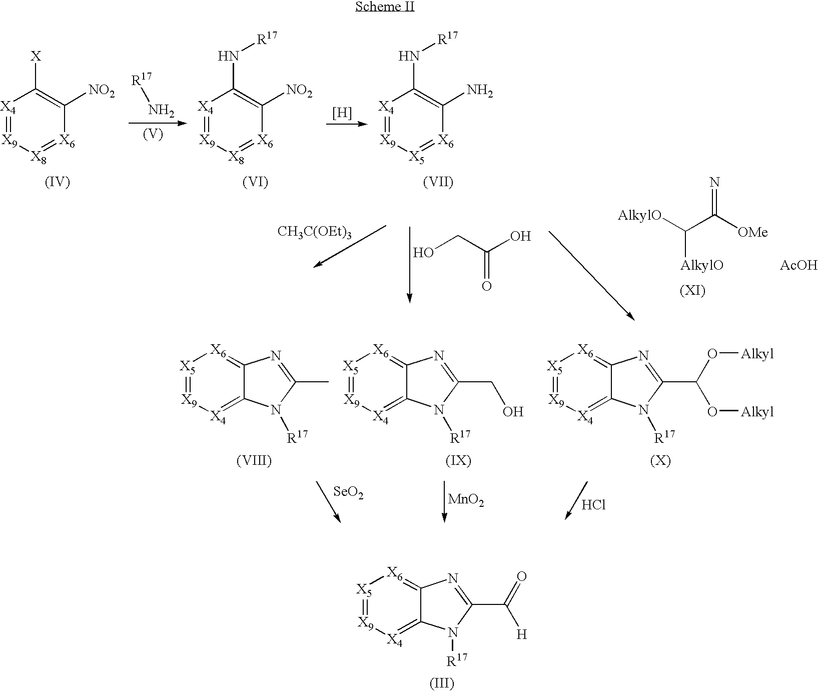

Scheme III illustrates the synthesis of compound of formula (XVIII), wherein R⁵ to R¹⁷ are defined as above and R is hydrogen or any one of the substituents R¹-R⁴ and R⁶ as defined in formula I. Boc-protected piperidinone (XII), either commercially available or readily prepared from commercial precursors, is treated with a suitable base, such as diethylisopropylamine, triethylamine and the like, in the presence of a triflic source such as triflic anhydride to form enol triflate of formula (XIII). Suzuki coupling of enol triflate (XIII) and boronic acid (XIV) with a catalyst such as palladium (0) tetrakis(triphenylphosphiine), palladium (II) acetate, allyl palladium chloride dimer, tris(dibenzylideneacetone)dipalladium (0), tris(dibenzylideneacetone)dipalladium (0) chloroform adduct, palladium (II) chloride or dichloro[1,1′-bis(diphenylphosphino)ferrocene]paladium (II) dichloromethane adduct, in the presence or absence of a base such as potassium phosphate, potassium acetate, sodium acetate, cesium acetate, sodium carbonate, lithium carbonate, potassium carbonate, cesium fluoride or cesium carbonate, preferably sodium carbonate, give olefin (XVI). This reaction is typically carried out in an inert solvent such as dimethyl ethylene glycol ether (DME), 1,4-dioxane, acetonitrile, methyl sulfoxide, tetrahydrofuran, ethanol, methanol, 2-propanol, or toluene, in the presence or absence of from about 1%-about 10% water, preferably about 5% water, with or without microwave assisted heating at a temperature from about 0° C. to about 200° C., preferably from about 60° C. to about 100° C. Hydrogenation of resultant olefin (XV) under hydrogen in the presence of a suitable catalyst, such as Pd/C, Pd(OH)₂ or PtO₂, yield aryl piperidine (XVI), Removal of Boc protecting group under acidic conditions, such as trifluoroacetic acid or HCl, give amines of formula (XVII), Amine (XVII) can then undergo reductive amination with aldehyde (III) as described in Scheme I, to give the compounds of formula (XVIII)

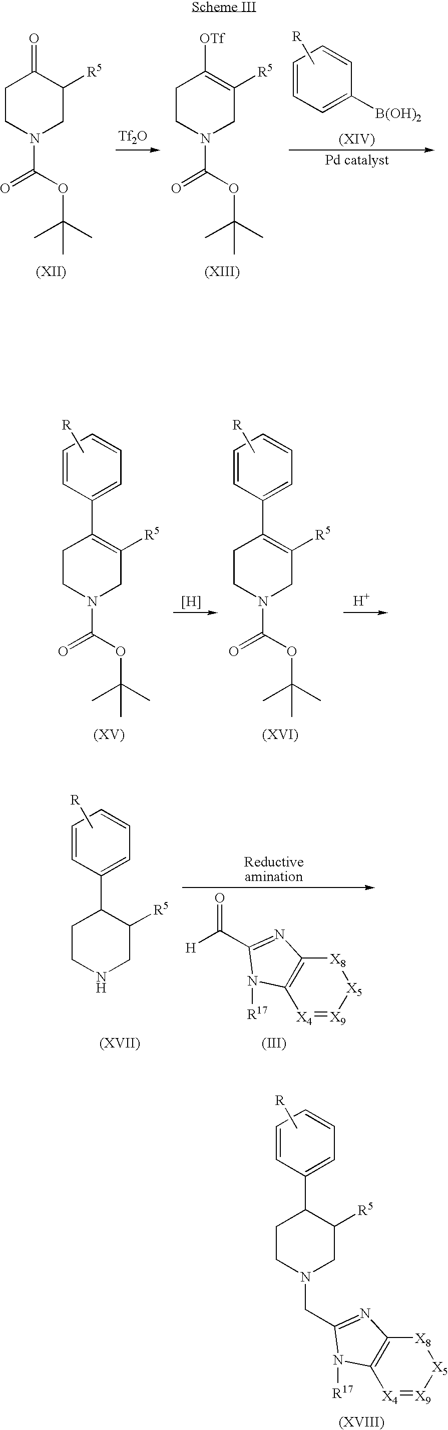

Alternatively, arylpiperidine of formula (XVII) can be synthesized as illustrated in Scheme V. Referring to Scheme V, piperidinone (XIX) with a suitable protecting group, such as benzyl, Boo or CBZ, can be treated with a lithium or aryl Grignard species of formula (XX) to yield alcohol of formula (XXI). Dehydration of alcohol (XXI) under strong acidic conditions, such as trifluoroacetic acid or aqueous HCl solution, yields a mixture of olefin isomers (XXIIa) and (XXIIb). Subsequent hydrogenation of the oelfin (XXIIa, b) using a suitable catalyst, such as Pd/C, PtO₂ or Pd(OH)₂, under hydrogen in a suitable solvent, such as ethanol, methanol or ethylacetate, followed by deprotection yield arylpiperidine of formula (XVII), which then can be further derivatized to give compound of formula (XVIII) as described in Scheme III. Alternatively, treating alcohol (XXI) with ethyl chloroformate yields carbonate (XXIII), which upon heating in a suitable high boiling point and inert solvent, such as decalin, give olefin of formula (XXIIb). Subsequent hydrogenation of the olefin and deprotection give aryl piperidine of formula (XVII),

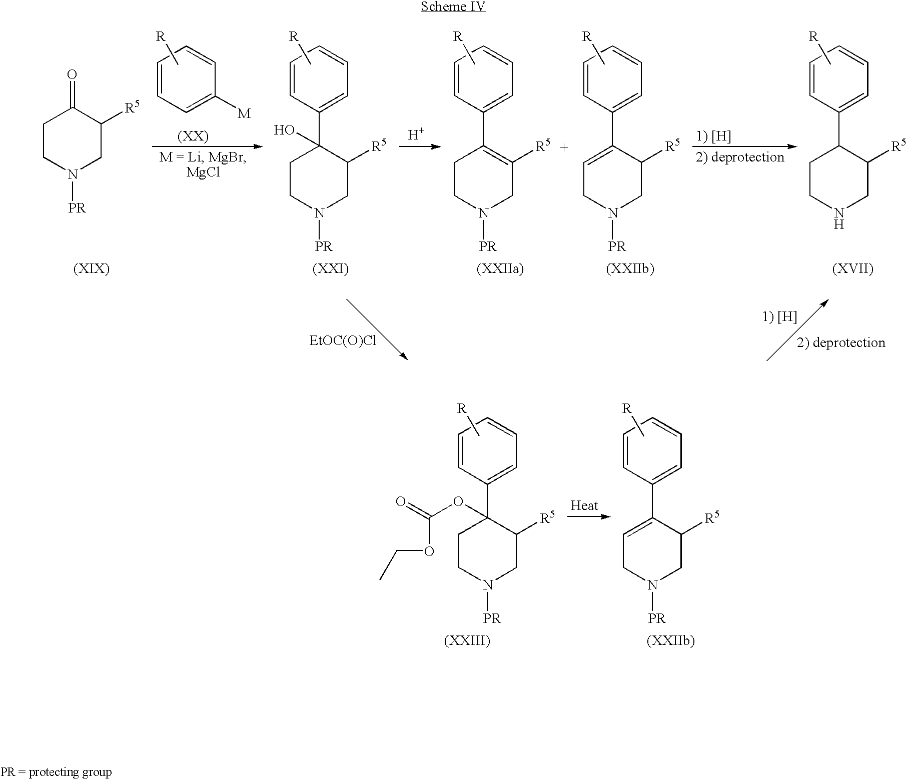

Scheme V illustrates the synthesis of compounds of formula (XXVII), wherein R⁵, R⁸, R⁹, R¹¹-R¹⁴ and R¹⁷ are defined as above. R is hydrogen or any one of the substituents R¹-R⁴ and R⁸ as defined in formula I. Bromopyridine of formula (XXIV), either commercially available or easily prepared from commercial sources, can be coupled with boronic acid of formula (XIV) to give aryl-pyridine (XXV). Suitable conditions for this Suzuki coupling reaction involve a catalyst, such as palladium (0) tetrakis(triphenylphosphine), palladium (II) acetate, allyl palladium chloride dimer, tris(dibenzylideneacetone)palladium (0), tris(dibenzylideneacetone)palladium (0) chloroform adduct, palladium (II) chloride or dichloro[1,1′-bis(diphenylphosphino)ferrocene]palladium (II) dichloromethane adduct, in the presence or absence of a base such as potassium phosphate, potassium acetate, sodium acetate, cesium acetate, sodium carbonate, lithium carbonate, potassium carbonate, cesium fluoride or cesium carbonate, preferably sodium carbonate. This reaction is typically carried out in an inert solvent such as dimethyl ethylene glycol ether (DME), 1,4-dioxane, acetonitrile, methyl sulfoxide, tetrahydrofuran, ethanol, methanol, 2-propanol, or toluene, in the presence or absence of from about 1% to about 10% water, preferably about 5% water, with or without microwave assisted heating at a temperature from about 0° C. to about 200° C. preferably from about 60° C. to about 100° C. Hydrogenation of the HCl salt of pyridine (XXV) under hydrogen in the presence of a suitable catalyst, such as Pd/C, Pd(OH)₂ or PtO₂, in a suitable solvent, such as ethanol, at elevated temperature, yield amine (XXVI). The resultant amine (XXVI) can then undergo reductive amination with aldehyde (II) as described in Scheme I, to give the compounds of formula (XXVII)

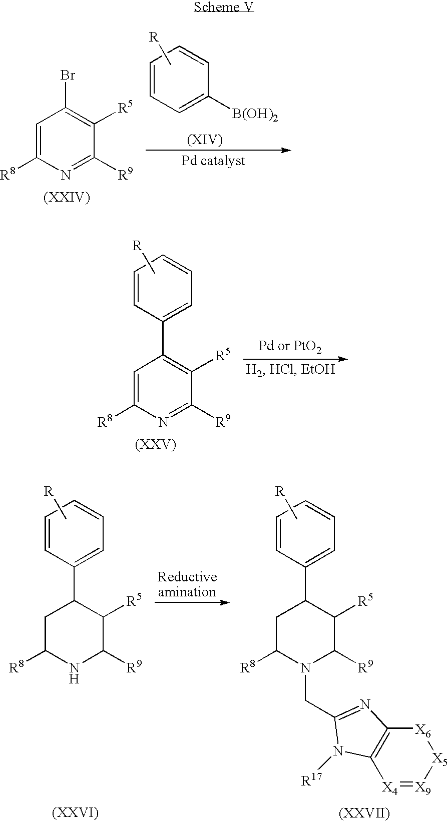

Scheme VI illustrates the synthesis of compounds of formula (XXXII), wherein R¹³-R¹⁴, R¹⁷ and R¹⁰¹ are defined as above. R is hydrogen or any one of the substituents R¹-R⁴ and R⁵ as defined in formula I. Referring to Scheme VI deprotection of the methoxy group of arylpiperidine (XXVIII) yield phenol of formula (XXIX). Phenol (XXIX) can be coupled with an alcohol of formula (XXX) in the presence of a suitable coupling reagent such as diethylazodicarboxylate (DEAD) and triarylphosphines, such as triphenylphosphine, in solvents such as THF or ether at or about room temperature, to produce the corresponding ether of formula (XXXI). The amine (XXXI) can then undergo reductive amination with aldehyde (III) as described in Scheme I, to give the compounds of formula (XXXII)

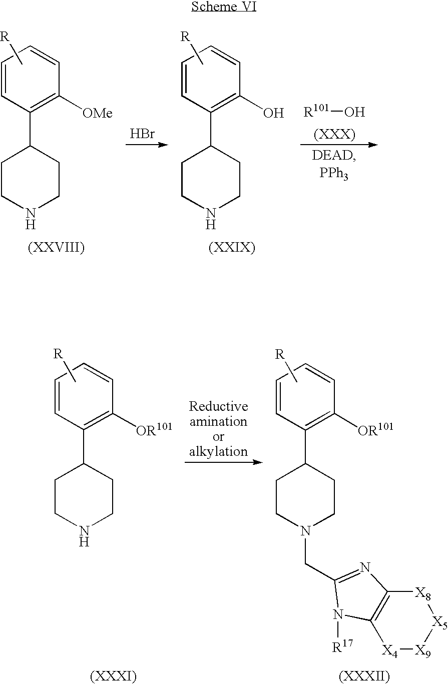

Scheme VII illustrates a synthesis of F— or —OH substituted piperidines, wherein R is hydrogen or any one of the substituents R¹-R⁴ and R⁸ as defined in formula I. Referring to Scheme VII, alcohol (XXXVIII) can be treated with a fluorinating reagent, such as diethylaminosulfurtrifluoride (DAST) or bis-(1-methoxyethyl)aminosulfurtrifluoride (BAST) in a suitable solvent such as methylene chloride, to give fluorinated compound of (XXXIX). Deprotection of Boc under acidic conditions yield 4-fluoro piperidine of formula (XL). For the synthesis of 3-fluoropiperidine (XLIII), olefin (XLI) can be converted to alcohol (XLII) via hydroboration reaction. A typical condition involves treating the substrate with borane dimethylsulfide complex, followed by hydrogen peroxide and sodium hydroxide aqueous solution. The resulting alcohol (XLII) can be deprotected under acidic condition to 3-hydroxyl piperidine (XLIV), or be fluorinated with DAST or BAST to give (XLIII), which upon deprotection to yield 3-fluoro piperidine (XLV). Reductive amination of (XL), (XLIV) or (XLV) with aldehyde (III) according to Scheme I will yield desired compounds of formula (I).

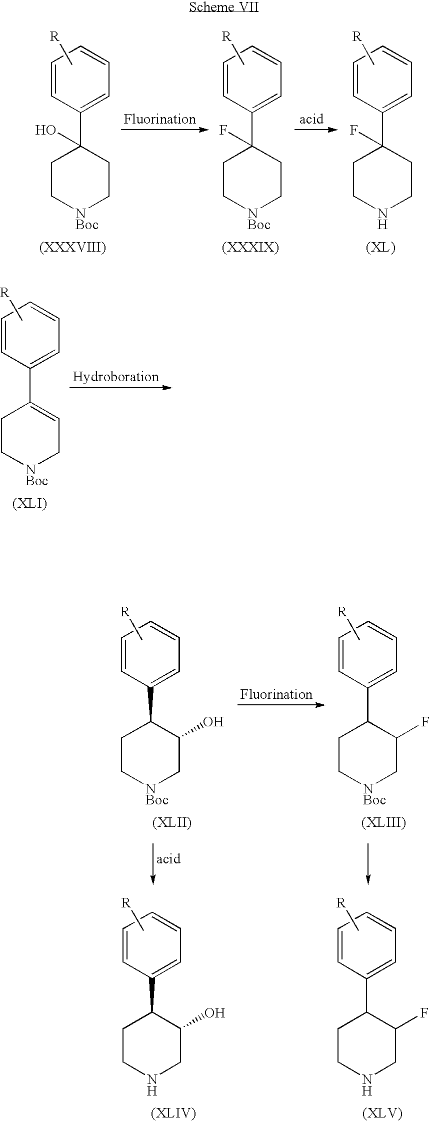

Scheme VIII illustrates the synthesis of compounds of formulae (LV) and (LVI), wherein R is hydrogen or any one of the substituents R¹-R⁴ and R⁸ as defined in formula I. Commercially available amino acid (XLV) can be protected as a carbamate, here illustrated by benzyloxycarbonyl derivative (XLVI). Carboxyl group can be converted to acid chloride, for example by treatment with oxalyl chloride in an inert solvent such as toluene optionally in the presence of catalytic amount of DMF, Acid chloride (XLVII) can be converted into aldehyde (L) directly by means of reducing conditions such as hydrogenation over palladium catalyst. Alternatively acid chloride (XLVII) can be converted into alkyl ester (XLVIII) by reaction with an excess of the corresponding alcohol. The selective reduction of ester (XLVIII) to alcohol (XLIX) can be achieved, for example, by reaction with sodium borohydride in an alcoholic solvent. Conversion of primary alcohol (XLIX) to (L) can be accomplished by well known oxidation conditions such as Swern oxidation and Dess-Martin oxidation. Spiroindoline derivatives (LII) can be prepared by reacting hydrazines (LI) with protected aminoaldehydes such as (L) in an inert solvent such as toluene, dichloromethane or acetonitrile in the presence or absence of acidic catalysts exemplified by trifluoroacetic acid or zinc chloride followed by treatment with reducing agents such as sodium borohydride. The free amino group of (LII) can be protected, for example as Boc (tert-butyloxycarbonyl) derivative illustrated by structure (LIII). Cbz group can be removed using reducing conditions such as hydrogenation over palladium catalyst to afford mono-protected derivative (LV). Cbz group removal can also be performed in a similar manner on the spiroindolines (LII) to afford diamines (LIV). The more reactive amino group of (LIV) can be selectively protected for example as Boc carbamate (LV).

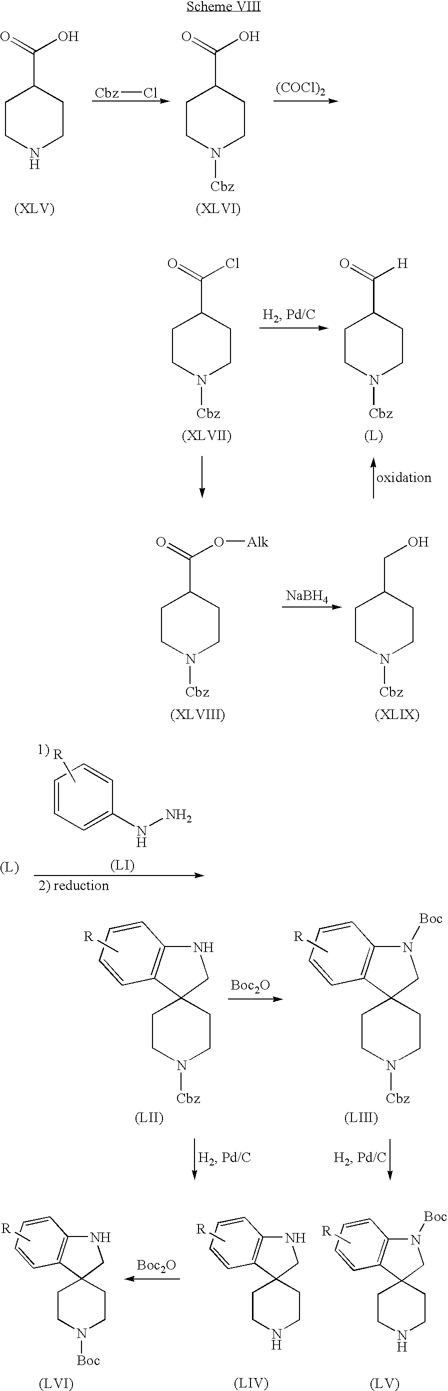

Scheme IX illustrates an alternative synthesis of compounds of formula (LVI), wherein R is hydrogen or any one of the substituents R¹-R⁴ and R⁶ as defined in formula I. (2-Fluoroaryl)acetonitriles (LVII) can be reacted with 2-chloro-N-(2-chloroethyl)-N-methylethanamine in the presence of a suitable base, such as, but not limited to, cesium carbonate, sodium hydride, potassium hexahydrodisilazide in solvents such as THF, DMF or DMSO to afford piperidines (LVIII). Spiroindoline compounds (LIX) can be obtained by reduction and spontaneous cyclization of (LVIII) using hydride reducing agents such as lithium aluminium hydride in solvents such as dimethoxyethane, dioxane or glyme optionally in the presence of alcohols such as methanol or ethanol. The free amino group can be protected as a carbamate derivative here illustrated by benzyloxycarbamate (LX) using conventional methods. Compounds of formula (LXI) can be obtained by selective demethylation by reaction with chloroethylchloroformate. The free amino group of (LXI) can be protected, for example as (tert-butyloxycarbonyl) derivative illustrated by structure (LXII), Cbz group can be removed using reducing conditions such as hydrogenation over palladium catalyst to afford monoprotected derivative (LVI).

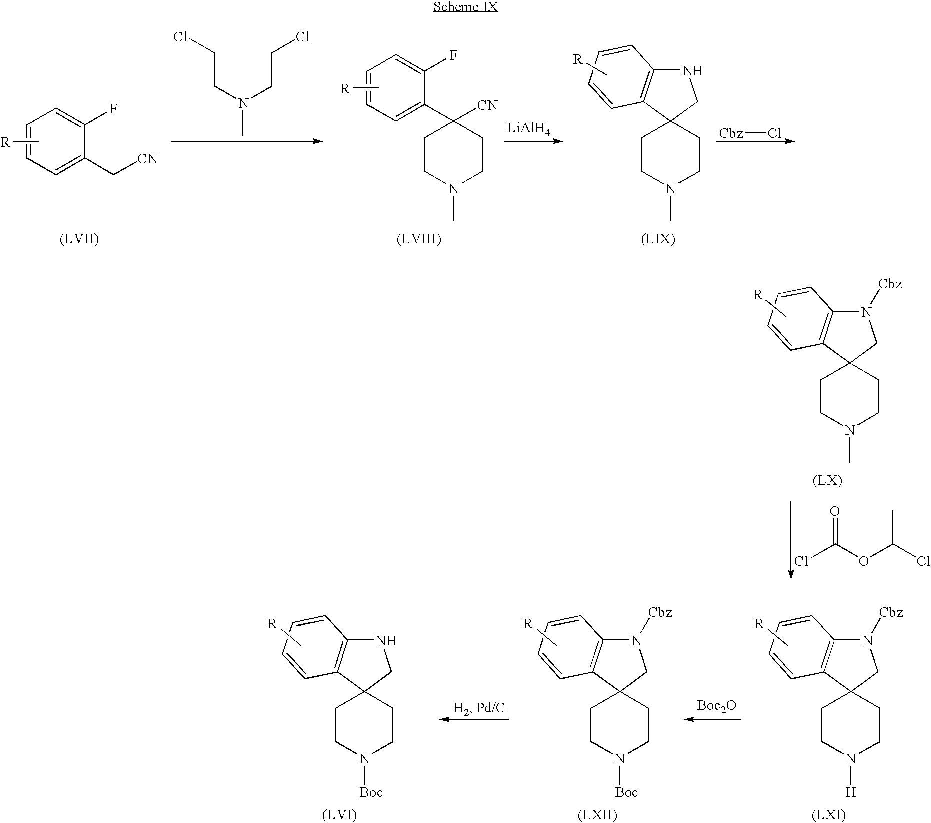

Scheme X illustrates the synthesis of compounds of formulas (LXV) and (LXVI) where R′ is hydrogen or optionally substituted alkyl such as C₁-C₆ alkyl, R″ is optionally substituted aryl, heteroaryl or alkyl such as C₁-C₅ alkyl and wherein R¹¹-R¹⁴ and R¹⁷ are defined as above. Spiroindoline derivative (LV) can undergo reductive amination with aldehyde (III), as described in Scheme I, to give the compounds of formula (LXIII). Boc group can be removed by treatment with acidic reagents such as hydrochloric or trifluoroacetic acids in a solvent such as ether, dioxane or methanol. The compounds (LXV) can be synthesized by treating secondary amine of formula (LXIV) with the corresponding aldehydes in the presence of suitable reducing agents such as NaBH(OAc)₃, Na(CN)BH₃, or formic acid in solvents such as methylene chloride, dichloroethane, DMF or THF, at about room temperature. Other suitable conditions for this transformation include treatment of the amine of formula (LXIV) with aldehydes in solvents such as methanol or ethanol at room temperature, followed by treatment with reducing agents such as NaBH₄ or NaCNBH₃, which also produce the desired compounds of formula (LXV). Alternatively, a compound of formula (LXV) can be synthesized by alkylating the amine of formula (LXIV) with the corresponding alkylating agent in the presence of a suitable base, such as, but not limited to, dimethylpropylamine, sodium carbonate, potassium carbonate, or sodium ethoxide, in solvents such THF, DMF or DMSO, at elevated temperature around 40° C. to 180° C. with or without microwave heating. Alternatively, amines (LXIV) can be converted to amides (LXVI) by treatment with the corresponding carboxylic acids in the presence of activating agents such as, but not limited to, HBTU, HATU, carbonyldiimidazole, DMC, HOBT, and DCC in the presence or absence of a suitable base, such as, but not limited to, diethylpropylamine, sodium carbonate, potassium carbonate. Amides (LXVI) can also be prepared by treatment of amines (LXIV) with the corresponding acid chlorides in the presence of a suitable base, such as, but not limited to, diethylpropylamine, sodium carbonate, potassium carbonate in solvents such as dichloromethane. THF, DMF or DMSO.

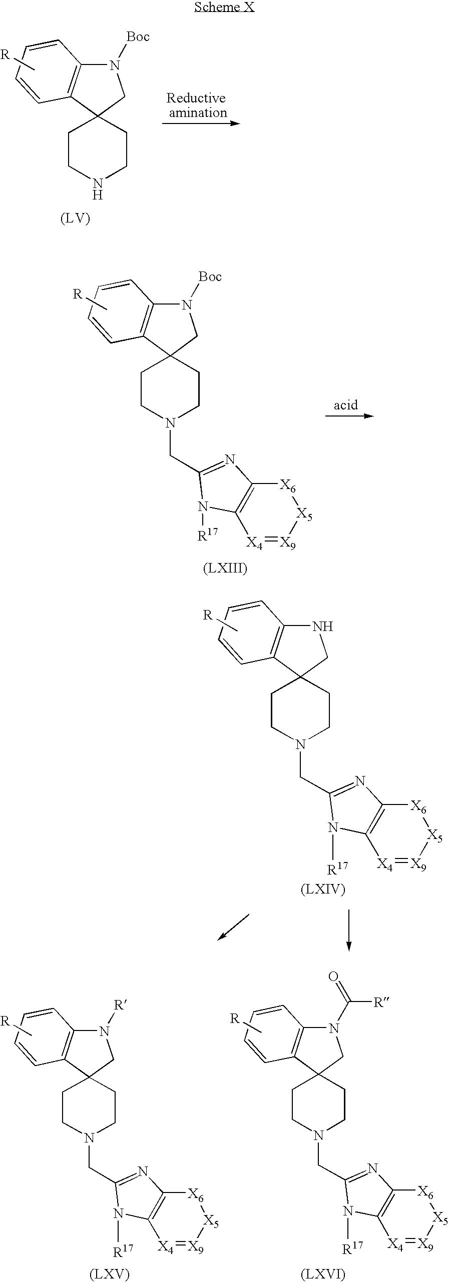

Scheme XI illustrates an alternative synthesis of compounds of formulae (LXV) and (LXVI) where R′ is hydrogen or optionally substituted alkyl such as C₁-C₆ alkyl, R″ is optionally substituted aryl, heteroaryl or alkyl such as C₁-C₆ alkyl and wherein R¹¹-R¹⁴ and R¹⁷ are defined as above. The compounds (LXVI) can be synthesized by treating secondary amine of formula (LVI) with the corresponding aldehydes in the presence of suitable reducing agents such as NaBH(OAc)₃, Na(CN)BH₃, or formic acid in solvents such as methylene chloride, dichloroethane, DMF or THF, at about room temperature. Other suitable conditions for this transformation include treatment of the amine of formula (LVI) with aldehydes in solvents such as methanol or ethanol at room temperature, followed by treatment with reducing agents such as NaBH₂ or NaCNBH₃, which also produce the desired compounds of formula (LXVI). Alternatively, a compound of formula (LVI) can be synthesized by alkylating the amine of formula (LXVI) with the corresponding alkylating agent in the presence of a suitable base, such as, but not limited to, diethylpropylamine, sodium carbonate, potassium carbonate, or sodium ethoxide, in solvents such THF, DMF or DMSO, at elevated temperature around 40° C. to 180° C. with or without microwave heating. Alternatively, amines (LVI) can be converted to amides (LXVIII) by treatment with the corresponding carboxylic acids in the presence of activating agents such as, but not limited to, HBTU, HATU, carbonyldiimidazole, DMC, HOBT, and DCC in the presence or absence of a suitable base, such as, but not limited to, diethylpropylamine, sodium carbonate, potassium carbonate. Amides (LXVIII) can also be prepared by treatment of amines (LVI) with the corresponding acid chlorides in the presence of a suitable base, such as, but not limited to, diethylpropylamine, sodium carbonate, potassium carbonate in solvents such as dichloromethane, THF, DM F or DMSO. Free amine derivatives of formulae (LXVII) and (LXIX) can be prepared by removal of the Boc group by treatment with acidic reagents such as hydrochloric or trifluoroacetic acids in a solvent such as ether or dioxane. Amines (LXVII) and (LXIX) can undergo reductive amination with aldehyde (III) as described in Scheme I, to give the compounds of formulas (LXV) and (LXVI).

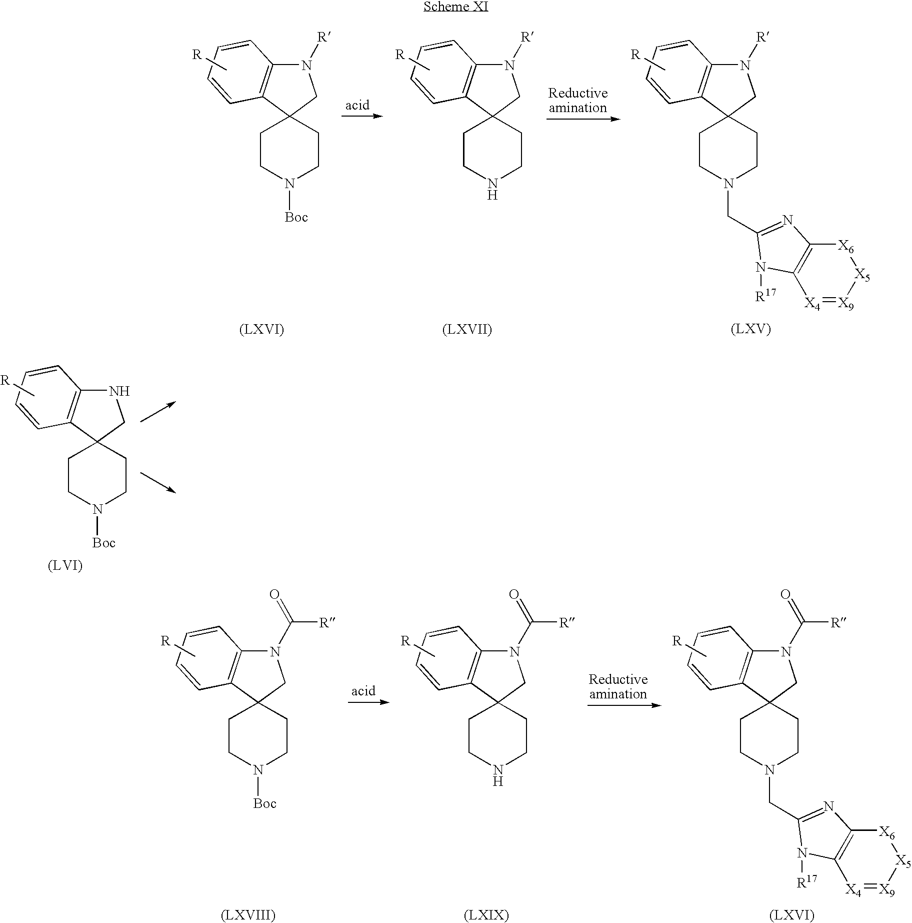

### WORKING EXAMPLES

The following illustrate the synthesis of various compounds of the present invention. Additional compounds within the scope of this invention may be prepared using the methods illustrated in these Examples, either alone or in combination with techniques generally known in the art.

### Preparation of Substituted Azabenzimidazole Aldehydes-(III)

### Preparation 1

### 1-Methyl-1H-imidazo[4,5-c]pyridine-2-carbaldehyde Dihydrochloride Dihydrate

4-Chloro-3-nitropyridine (70.0 g, 0.44 mol) was suspended in chloroform (280 mL) under stirring for 15 min. The suspension was cooled in an ice bath and diluted with ethanol (280 mL). Aqueous 40% w/w solution of methylamine (98.2 mL) was added dropwise to this mixture under vigorous stirring and cooling. The reaction mixture was stirred at room temperature for 2 h and allowed to stay overnight. The mixture was diluted with chloroform (200 mL). The yellow precipitate was separated and washed with hot chloroform (400 mL). The filtrate was concentrated under reduced pressure to dryness, and the residue was dissolved in chloroform (800 mL). The solution was washed with water (2×800 mL) and dried over Na₂SO₄. The solvent was removed under reduced pressure and the solid residue was recrystallized from acetone (760 k mL) to give compound N-Methyl-3-nitropyridin-4-amine (38.8 g. 57.4%, 0.25 mol) as a yellow crystalline solid.

N-Methyl-3-nitropyridin-4-amine (49.43 g, 0-323 mol) was suspended under vigorous stirring in methanol (500 mL). Activated carbon (2.0 g) was added to the suspension, which was refluxed for 2.5 h and then allowed to stay overnight at room temperature. The reaction apparatus was flushed with dry nitrogen, and the catalyst (Pd/C 10%, 4.9 g) was added to the mixture Hydrogen was bubbled through the mixture for 21 h under stirring at room temperature. The obtained mixture was passed through Celite (upper layer, 3 cm) and silica gel (lower layer, 5 cm, diameter 13 cm) to remove the catalyst. The layers were washed with methanol (3×300 mL). The filtrate was concentrated under reduced pressure to afford N⁴-Methylpyridine-3,4-diamine (39.54 g, 99.5:%, 0.32 mol) as a brown crystalline solid. The obtained product was used for the next stage without additional purification.

Sodium (12.0 g, 0.52 mol) was dissolved in anhydrous methanol (450 mL). A solution of diethoxyacetonitrile (75 g, 0.58 mol) in anhydrous methanol (210 mL) was added dropwise to the solution of sodium methoxide under stirring. The obtained mixture was stirred at room temperature for 2 h and allowed to stay overnight. The reaction mixture was concentrated under reduced pressure, and the residue was dissolved in water (300 mL). The solution was shaken with chloroform (200 mL), and the layers were separated. The aqueous layer was additionally treated with chloroform (2×150 mL). The organic extracts were combined and dried over Na₂SO₄. The solvent was removed to give Methyl 2,2-Diethoxyethanimidoate (63.82 g, 68% 0.4 mol) as a pale-yellow liquid.

N⁴-Methylpyridine-3,4-diamine (39.54 g, 0.32 mol) was added to a solution of Methyl 2,2-Diethoxyethanimidoate (52.02 g, 0.323 mol) in anhydrous methanol (150 mL). The obtained mixture was diluted with anhydrous methanol (50 mL) and cooled in an ice bath. 4 M HCl in dioxane (86 mL) was added dropwise to the mixture under stirring for 15 min. The mixture was refluxed for 5 h and concentrated under reduced pressure. The residue was dissolved in a mixture of chloroform (300 mL)l and water (300 mL). The layers were separated, and the aqueous layer was treated with chloroform (3×250 mL) to extract the product. The extracts were combined, dried over Na₂SO₄, and evaporated to give a red mass (45 g). The latter was chromatographed (silica gel, chloroform/ethanol 40:1). The solvent was removed to give 2-(Diethoxymethyl)-1-methyl-1H-imidazo[4,5-c]pyridine (31.85 g, 42%, 0.135 mol) as a red liquid.

2-(Diethoxymethyl)-1-methyl-1H-imidazo[4,5c]pyridine (36.9 g, 0.157 mol) was mixed under vigorous stirring with 4 M HCl (103 mL). The reaction mixture was stirred at 60° C. for 3 h and evaporated to dryness. The residue was mixed with dioxane (150 mL), and the mixture was concentrated under reduced pressure to remove residual water. The operation was repeated to give a crystalline solid, which was triturated with anhydrous ether (150 mL). The pale-yellow precipitate was separated by filtration and washed with ether to furnish 1-Methyl-1H-imidazo[4,5-c]pyridine-2-carbaldehyde Dihydrochloride Dihydrate (36.35 g, 92%). ¹H NMR (400 MHz. D₂O) δ 9.28 (s, 1H), 8.62 (d, 1H), 8.21 (dd, 1H), 6.50 (s, 1H), 4.21 (s, SH); MS (m/z) 162.1.

### Preparation 2

### 3-Methyl-3H-imidazo[4,5-b]pyridine-2-carbaldehyde Hydrochloride Hydrate

2-chloro-3-nitropyridine 70.0 g, 0.44 mol) was dissolved in recently distilled acetonitrile (400 mL) under stirring Sodium acetate (55.2 g 0.67 mol) and 30%; aqueous solution of methylamine (111 mL) were added under vigorous stirring. The obtained suspension was stirred at room temperature for 30 min, refluxed for 1 h, and kept overnight at room temperature. The yellow reaction mixture was concentrated under reduced pressure to remove approximately 300 mL of the solvent. The residue was diluted with 20% aqueous solution of K₂CO₃, (1 L) under stirring. The yellow precipitate was filtered off washed with water (3°×200 mL), and dried to afford N-Methyl-3-nitropyridin-2-amine in 86% (58.14 g, 0.38 mol) yield as bright yellow crystals.

N-Methyl-3-nitropyridin-2-amine (58.14 g, 0.38 mol) was dissolved in 1,2 dimethoxyethane (400 mL) under vigorous stirring. The obtained solution was refluxed with activated charcoal (2.9 g) for 2 h and kept overnight at room temperature. The reaction apparatus was flushed with dry nitrogen, and the catalyst (Pd/C 10%, 1.75 g) was added. The mixture was heated to 40° C. Hydrazine monohydrate (54 mL, 1.08 mol) was added dropwise to the suspension within 2 h. The obtained mixture was refluxed for 2 h, cooled, and passed through Celite (upper layer, 3 cm) and silica gel (lower layer, 5 cm, diameter 13 cm) to remove the catalyst. The layers were washed with 1,2-dimethoxyethane (300 mL). The filtrate was concentrated under reduced pressure to afford N²-methylpyridine-2,3-diamine in 98% (46.2 g) yield as a brown crystalline solid. The product was used for the next stage without additional purification.

N²-methylpyridine-2,3-diamine (44.33 g, 0.36 mol) was dissolved in 1,2-dimethoxyethane ('200 mL). Methyl 2,2-Diethoxyethanimidoate (63.8 g, 0.4 mol) and glacial acetic acid (21.6 g, 0.36 mol) were added to the solution under stirring. The obtained mixture was stirred at room temperature for 7 h, then refluxed for 40 min. The mixture was concentrated under reduced pressure to dryness, and the residue was purified by chromatography on a silica gel column (ethyl acetate/hexane 1:2) to furnish 2-(Diethoxymethyl)-3-methyl-3H-imdazo[4,5-b]pyridine in 65.6% (55.53 g, 0.236 mol) yield as a yellow liquid.

2-(Diethoxymethyl-3-methyl-3H-imidazo[4,5-b]pyridine (53.54 g, 0.228 mol) was mixed under vigorous stirring with 4 M HCl (150 mL). The reaction mixture was stirred at 60° C. for 3 h and evaporated to dryness. The residue was mixed with dioxane (1650 mL) and the mixture was concentrated under reduced pressure to remove residual water. The operation was repeated to give a crystalline solid, which was triturated with anhydrous ether (150 mL). The yellow precipitate was filtered off and washed with ether to furnish 3-Methyl-3H-imidazo[4,5-b]pyridine-2-carbaldehyde Hydrochloride Hydrate in 100% (45.4 g) yield. ¹H NMR (400 MHz, D₂O) δ 8.61 (d, 1H), 8.25 (d, 1H), 7.64 (dd, 1H), 6.54 (s, 1H), 4.20 (s, 3H); MS (m/z) 162.1.

### Preparation 3

### 1-Methyl A H-imidazo[4,5-b]pyridine-2-carbaldehyde Hydrochloride Hydrate

3-Methoxy-2-nitropyridine (25.15 g, 0.163 mol) and 33% w/w solution of methylamine in ethanol (82 mL, 0.65 mol) were placed into a reactor vessel of MILESTONE Microwave Labstation. The reaction mixture was treated with microwave radiation under stirring at an internal temperature of 105° C. for 4.5 h. The reaction mixture was cooled and diluted with chloroform (200 mL). The obtained suspension was concentrated under reduced pressure to dryness, and the residue was purified by column chromatography (silica gel, 1 kg, chloroform/1,2-dimethoxyethane, 50:1, ˜3 L). The eluate was evaporated to dryness. The residue was purified by column chromatography (silica gel, 1 kg, chloroform, 5 L→chloroform/1,2-dimethoxyethane, 200:1, 7 L). The second fraction containing the product was concentrated under reduced pressure, and the residue was recrystallized from 1,2-dimethoxyethane/hexane mixture 1:1 to give N-Methyl-2-nitropyridin-3-amine (14.18 g, 57%, 0.093 mmol).

N-Methyl-2-nitropyridin-3-amine (14.1 g, 0.092 mol) vas suspended under vigorous stirring in 1,2-dimethoxyethane/methanol mixture (1:1, 400 mL). The reaction apparatus was flushed with dry nitrogen. The catalyst (Pd/C 10%, 1.4 g) was added to the mixture. Hydrogen was bubbled through the suspension for 7 h. The reaction mixture was diluted with chloroform (300 mL) and passed through a fitter with Celite (upper layer, 3 cm) and silica gel (lover layer, 5 cm, diameter 13 cm) to remove the catalyst. The layers were washed with chloroform/methanol mixture (1:1, 500 mL). The solvent was removed under reduced pressure, and the residue was mixed with acetonitrile. The mixture was concentrated under reduced pressure to give N-Methylpyridine-2,3-diamine (11.05 g, 97%, 0.09 mol). The product was used for the next stage without additional purification.

N-Methylpyridine-2,3-diamine (11.0 g. 0.089 mol),was dissolved in 1,2-dimethoxyethane (300 mL). Methyl 2,2-diethoxyethanimidoate (31.0 g, 0.19 mol) and glacial acetic acid (10 mL) were added to the solution under stirring. The obtained mixture was stirred at room temperature for 3 h, then refluxed for 5 h. p-Toluenesulfonic acid monohydrate (0.1 g) was added to the reaction mixture, which was refluxed for 7 h. The mixture was concentrated under reduced pressure, and the residue was diluted with toluene (300 mL). The mixture was refluxed for 7 h, cooled, and mixed with a solution of Na₂CO₃ (20 g) in water (500 mL). The product was extracted with ethyl acetate (3×300 mL) and chloroform (400 mL). The combined extracts were dried over MgSO₄ and concentrated under reduced pressure to give a dark brown solid, which was chromatographed on silica gel (chloroform/1,2-dimethoxyethane 1:1, 900 mL). 2-(diethoxymethyl)-1-methyl-1H-imidazo[4,5-b]pyridine was obtained (8.3 g, 39.5%, 0.35 mol).

2-(Diethoxymethyl)-1-ethyl-1H-imidazo[4,5-b]pyridine (8.3 g, 0.035 mol) was mixed under vigorous stirring with 4 M HCl (25 mL). The reaction mixture was stirred at 57-58° C. for 3 h and evaporated to dryness. The residue was mixed with dioxane (150 mL), and the mixture was concentrated under reduced pressure to remove residual water. The operation was repeated to give a crystalline solid, which was triturated with anhydrous ether (150 mL). The yellow precipitate was separated by filtration and washed with ether (3×250 mL) to furnish 1-Methyl-1H-imidazo[4,5-b]pyridine-2-carbaldehyde hydrochloride hydrate (7.3 g, 96%, 0.034 mol). ¹H NMR (400 MHz, D₂O) δ 8.70 (d, 1H), 8.67 (d, 1H), 7.87 (dd, 1H), δ 6.52 (s, br, 1H), 4.23 (s, 3H); MS (m/z) 162.1.

### Preparation 4

### 3-Methyl-3H-imidazo[4,5-c]pyridine-2-carbaldehyde

To a solution of 3-bromo-4-nitropyridine N-oxide (0.5 g, 2.28 mmol) in 2 ml ethyl alcohol, methylamine (33% weight in ethyl alcohol) (0.57 ml, 4.56 mmol) was added and the mixture was heated by microwave under 90° C. for 20 minutes. The mixture was concentrated under reduced pressure to yield Methyl-(4-nitro-1-oxy-pyridin-3-yl)-amine (0.38 g); MS 170.1.

To a solution of Methyl-(4-nitro-1-oxy-pyridin-3-yl)-amine (0.38 g, 2.28 mmol) in 4 ml acetic acid, iron powder (383 mg, 6.849 mmol) was added and the mixture was heated by microwave under 180° C. for 30 minutes. The mixture was basified to pH 12 by 1 N NaOH solution. The resulting mixture was extracted by DCM, dried over Na₂SO₄, filtered and the solvent was removed in vacuo. The residue purified using silica gel chromatography (0% to 10% MeOH/CH₂Cl₂) to yield 2,3-dimethyl-3H-imidazo[4,5-c]pyridine (0.1 g); GC-MS 147.

To a solution of 2,3-dimethyl-3H-imidazo[4,5-c]pyridine (0.31 g 2.14 mmol) in 3.5 ml 1,4-dioxine, selenium dioxide (356 mg, 3.21 mmol) was added and the mixture was heated by microwave under 150° C. for 30 minutes. The mixture was filtered and the solvent as removed in vacuo. The residue purified using silica gel chromatography (0% to 10% MeOH/CH₂Cl₂) to yield 3-methyl-3H-imidazo[4,5-c]pyridine-2-carbaldehyde (0.15 g); GC-MS 161.

### Preparation 5

### 5,6-Dihydro-4H-imidazo[4,5,1-ij]-1,7-naphthyridine-2-carbaldehyde

A 500 mL high pressure Parr vessel, was charged with 1,7-naphthyridin-8-amine (Aldrich, 6.0 g, 41.3 mmol), Pd(OH)₂/C (20%) (Aldrich, 3.0 g), conc. HCl (8.26 mL, ˜83 mmol), ethanol (100 mL). The mixture was closed and hydrogenated in Parr apparatus at 40 psi for 5 days. LCMS showed completed reaction. The mixture was filtered through celite and evaporated to give yellow residue of HCl salt. This residue was dissolved in 20 mL of water, basified to pH 12 with NaOH, and extracted with DCM (4×100 mL). The extract was dried over Na₂SO₄ and evaporated to give 6.00 g (97%) of 1,2,3,4-tetrahydro-1,7-naphthyridin-8-amine as a tan solid. LCMS (M+H): 150.3; ¹H NMR (300 MHz CDCl₃): δ 7.28 (d, J=5.28 Hz, 1H), 6.45 (d, J=5.1 Hz, 1H), 4.14 (br., 2H), 3.32 (m, 2H), 3.20 (br., 1H), 2.69 (m, 2H), 1 90 (m, 2H) ppm.

A 250 mL high pressure vessel, equipped with a magnetic stirring bar, was charged with 1,2,3,4-tetrahydro-1,7-naphthyridin-8-amine (6.00 g, 40.27 mmol), methylorthoformate (60 mL), and 99% formic acid (4 mL). The mixture was closed and heated at 100° C. for 14 h. Then the mixture was evaporated under vacuum and the dark residue was mixed with 10 mL of sat Na₂CO₃. The mixture was extracted with DCM (3×100 mL). The extract was dried over Na₂SO₄ and evaporated. The crude dark oil was purified by column (DCM 46%, ether 46%, MeOH 5%, Et₃N 3%, Rf=0.18 in the same system) to give 5.13 g (80%) of 5,6-dihydro-4H-imidazo[4,5,1-ij]-1,7-naphthyridine as a yellow solid. LCMS (M+H): 160.1; ¹H NMR (300 MHz, CDCl₃): δ 8.47 (d, J=4.89 Hz, 1H), 8.06 (s, 1H), 6.99 (d, J=4.89 Hz, 1H), 4.30 (t, J=5.75 Hz, 2H), 3.02 (t, J=6.03 Hz, 2H), 2.30 (m, 2H) ppm.

A 500 mL, 3-neck round bottomed flask, equipped with a magnetic stirring bar, nitrogen gas inlet, thermometer, and a septum, was charged with 5,6-dihydro-4H-imidazo[4,5,1-ij]-1,7-naphthyridine (5 42 g, 34.09 mmol) and anhydrous THF (220 mL). The suspension was heated to completely dissolve the materials then cooled to RT to form fine suspension. This suspension was cooled to −75° C. and then LDA (2M in heptane/THF/ethylbenzene, 18.76 mL, 37.51 mmol) was added dropwise to keep temperature below −60° C. The mixture was stirred at −70° C. for 3 h, then anhydrous DMF (7.95 mL, 102.26 mmol) was added over 5 min at temperature below −60° C. and slowly warmed to RT, then stirred at room temperature for 12 h. The mixture was cooled with an ice bath and then saturated aqueous solution of NaH₂PO₄ was added until pH=8.0−8.5. The mixture was extracted with DCM (4×300 mL), the extract was dried over Na₂SO₄ and evaporated to give crude residue, which was recrystallized by dissolving in 12 mL of MeOH, then addition of 70 mL of EtOAc followed by 70 mL of hexane. The precipitate was filtered and dried to give 5.5 g (85%) of 5,6-dihydro-4H-imidazo[4,5-ij]-1,7-naphthyridine-2-carbaldehyde as a yellow solid. LCMS (M+H): 188.4: ¹H NMR (300 MHz, CDCl₃) δ 10.18 (s, 1H), 8.65 (d, J=4.71 Hz, 1H), 7.13 (d, J=4.71 Hz, 1H), 4.67 (t J=583 Hz, 2H), 3.06 (t, J=6.12 Hz, 2H), 2.33 (m, 2H) ppm.

### Preparation Methods of Substituted Piperidine Templates (II)

For each preparation method, a representative synthesis is described. Other templates prepared through a similar synthetic sequence are listed in tables followed the description.

### Method A (Scheme III)

### Triflate Coupling/Hydrogenation

### Preparation 6

### 4-(4-Fluorophenyl)pyperidine hydrochloride

To a stirred solution of diisopropylamine (7 ml) in THF (150 ml) at −78° C. as added a solution of n-butyl lithium in hexanes (20 ml, 2.5 M). After 1 h tert-butyl 4-oxo-1-piperidinecarboxylate (10 g) was added. After an additional 1.5 h N-phenyltrifluoromethanesulfonimide (19.65 g) was added and the mixture was allowed to warm to room temperature. After stirring for 16 h the solvent was removed under reduced pressure and the resulting residue was used in the next step without purification.

A mixture of tert-butyl 4-trifluoromethanesulfonate-1-(1,2,3,6-tetrahydropyridine) carboxylate (8.3 g, crude), 4-fluorophenylboronic acid (3.5 g) and tetrakis(triphenylphosphine)palladium(0) (2.89 g) in a mixture of ethanol (85 ml) and water (15 ml) was stirred at 90° C. After 16 h the solvents were removed under reduced pressure, water was added and the mixture was extracted with ethyl acetate. The combined organics were washed with brine, dried over sodium sulfate, filtered and concentrated under reduced pressure. Purification by flash chromatography using a silica gel column and eluting with a gradient of 0% to 20% ethylacetate in hexanes gave 3.2 g of tert-butyl 4-(4-fluorophenyl)-1-(1,2,3,6-tetrahydropyridine) carboxylate as a brownish oil: ¹H NMR (400 MHz, (CD3CD) δ 1.47 (s, 9H), 2.49 (m, 2H), 3.61 (m, 2H), 4.03 (m, 2H), 6.04 (m, 1H), 7.04 (t, 1H), 7.23-7.44 (m, 3H).

A mixture of tert-butyl 4-(4-fluorophenyl)-1-(1,2,3,6-tetrahydropyridine) carboxylate (3.2 g) and 10% Pd on carbon (60 mg) in ethanol (20 ml) was shaken in a Parr apparatus under 40 psi of hydrogen. After 16 h the mixture was purged with nitrogen, filtered through celite, and concentrated under reduced pressure to give 3.2 g of tert-butyl 4-(4-fluorophenyl)-1-piperidinecarboxylate as a yellow oil: ¹H NMR (400 MHz, CD3OD) δ 1.47 (s, 9H), 1.49-1.62 (m, 2H), 1.79 (d, 2H), 2.66-2.74 (m, 1H), 2.85 (m, 2H), 4.18 (m, 2H), 6.99 (t, 1H), 7.18-7.38 (m, 3H).

A solution of tert-butyl 4-(4-fluorophenyl)-1-piperidinecarboxylate (3.2 g) in 4M HCl/dioxane (10 ml) was stirred at room temperature. After 2 h the mixture was concentrated under reduced pressure to give 2.5 g of 4-(4-fluorophenyl)pyperidine hydrochloride as a white solid, ¹H NMR (400 MHz, CD3OD) δ 1.82-1.93 (m, 2H), 2.03-2.08 (m, 2H), 2.88-2.94 (m, 1H), 3.09-3.16 (m, 2H), 3.47-3.50 (m, 2H), 7.05 (t, 1H), 7.23-7.39 (m, 3H); MS (m/z) 180.1.

The 4-substituted piperidines shown in Table 1 were prepared as above starting with the reaction of tert-butyl 4-trifluoromethanesulfonate-1-(1,2,3,6-tetrahydropyridine) carboxylate and the appropriate arylboronic acid:

TABLE 1

| NAME | m/z |
|---|---|
| 4-(5-fluoro-2-methoxyphenyl)piperidine hydrochloride | 210.1 |
| 4-(3,5-dimethylphenyl)piperidine hydrochloride | 190.2 |
| 4-(2-(1-hydroxyethyl)phenyl)piperidine hydrochloride | 206.2 |
| 4-(3-methylphenyl)piperidine hydrochloride | 176.2 |
| 4-(4-trifluoromethoxyphenyl)piperidine hydrochloride | 246.1 |
| 4-(3,4-dimethylphenyl)piperidine hydrochloride | |
| 4-(2,5-dimethylphenyl)piperidine hydrochloride | 190.1 |
| 4-(3,5-di(trifluoromethyl)phenyl)piperidine hydrochloride | 298.3 |
| 4-(2,4-difluorophenyl)piperidine hydrochloride | 198.2 |
| 4-(4-fluoro-2-methylphenyl)piperidine hydrochloride | 194.2 |
| 4-(2-methylsulfonylphenyl)piperidine hydrochloride | |
| 4-(4-isopropoxyphenyl)piperidine hydrochloride | |
| 4-(2-trifluoromethoxyphenyl)piperidine hydrochloride | 246.3 |
| 4-(4-ethylphenyl)piperidine hydrochloride | |
| 4-(3-fluoro-4-methoxyphenyl)piperidine hydrochloride | |
| 4-(3,5-difluorophenyl)piperidine hydrochloride | 198.2 |
| 4-(2-fluoro-6-methoxyphenyl)piperidine hydrochloride | |
| 4-(4-ethoxyphenyl)piperidine hydrochloride | 206.2 |
| 4-(4-methylphenyl)piperidine hydrochloride | 176.2 |
| 4-(2,3-difluorophenyl)piperidine hydrochloride | 198.2 |
| 4-(4-fluoro-2-methoxyphenyl)piperidine hydrochloride | 210.1 |

### Methods B (Scheme III)

### Triflate coupling/PtO₂-hydrogenation

### Preparation 7

### 4-(4-Chloro-3-fluorophenyl)piperidine hydrochloride

tert-Butyl 4-(4-chloro-3-fluorophenyl)-1-(1,2,3,6-tetrahydropyridine) carboxylate was prepared following the first two steps of Preparation 13 using 4-chloro-3-fluorophenyl boronic acid. A mixture of tert-butyl 4-(3-chloro-4-fluorophenyl)-1-(1,2,3,6-tetrahydropyridine) carboxylate (465 mg, 1.49 mmol) and PtO₂ (20 mg) in methanol (8 ml) was shaken in a Parr apparatus under 45 psi of hydrogen. After 1 h the mixture was purged with nitrogen, filtered through celite, and concentrated under reduced pressure to give 443 mg of tert-butyl 4-(3-chloro-4-fluorophenyl)-1-piperidinecarboxylate as a yellow oil.

A solution of tert-butyl 4-(4-Chloro-3-fluorophenyl)-1-piperidinecarboxylate (886 mg) in 4N HCl/dioxane (3 ml) was stirred at room temperature. After 4 h the mixture was concentrated under reduced pressure to give 749 mg of 4-(4-chloro-3-fluorophenyl)pyperidine hydrochloride; MS (m/z+CH₃CN) 255, 257.

The following 4-substituted piperidines were prepared as above starting with the reaction of tert-butyl 4-trifluoromethanesulfonate-1-(1,2,3,6-tetrahydropyridine) carboxylate and the appropriate arylboronic acid:

TABLE 2

| NAME | m/z |
|---|---|
| 4-(3-Chloro-4-fluoro-phenyl)piperidine hydrochloride | 214.2 |
| 4-(2,4-difluorophenyl)piperidine hydrochloride | 198.2 |
| 4-(4-Chloro-2-fluoro-phenyl)piperidine hydrochloride | 214.2 |
| 4-(2-Chloro-4-fluorophenyl)piperidine hydrochloride | 214.2 |

### Method C(Scheme IV)

### Orqanolithium/Grignard Addition

### Preparation 8

### 4-(4-trifluoromethylphenyl)piperidine hydrochloride

A solution of 1-bromo-4-(trifluoromethyl)benzene (238.5 g, 1.06 mol) in anhydrous THF (500 mL) was added dropwise to a stirred solution of n-butyllithium (508 mL of a 2.5 M solution in hexanes, 1.27 mol) in anhydrous tetrahydrofuran (1.0 L) at −60° C. under an atmosphere of argon. The resultant reaction mixture vas stirred at −60° C. for 1 h and then a solution of 1-benzylpiperidin-4-one in anhydrous tetrahydrofuran (600 mL) was added dropwise. The reaction mixture was allowed to with warm to 0° C. and was stirred at this temperature for 2 h before being made acidic with the addition of concentrated hydrochloric acid. The two layers were separated and the aqueous layer was basified with concentrated ammonium hydroxide and extracted with diethyl ether (2<500 mL). The organic fraction was then dried (MgSO₄) and concentrated under reduced pressure to a thick slurry, and the resultant solid was filtered, washed with hexane, and air-dried to afford 1-benzyl-4-[4-(trifluoromethyl)phenyl]piperidin-4-ol (265 g, 75%); R_{f} 0.04 (20% ethyl acetate in hexane).

A solution of 1-benzyl-4-[4-(trifluoromethyl)phenyl]piperidin-4-ol (123.5 g, 0.37 mol) in trifluoroacetic acid (750 mL) was heated at reflux over the weekend. The reaction mixture was then cooled to room temperature and concentrated under reduced pressure. Dichloromethane (1.0 L) and water (250 mL) were added to the residue and the pH of the solution was adjusted to 9 with the addition of concentrated ammonium hydroxide. The mixture was stirred at room temperature for 1 h. the organic phase was separated, and the aqueous phase was further extracted with dichloromethane (250 mL). The combined organic fractions were washed with water (250 mL), dried (MgSO₄), and the solvent was removed under reduced pressure to afford 1-benzyl-4-[4-(trifluoromethyl)phenyl]-1,2,3,6-tetrahydropyridine (115.5 g 98%) as an oil that solidified upon standing to give a granular beige solid; R_{f} 0.60 (7.8 ethyl acetate/hexane).

A solution of 1-benzyl-4-[4-(trifluoromethyl)phenyl]-1,2,3,6-tetrahydropyridine (100 g, 0.315 mol) in methanol (600 mL) was treated with palladium on carbon (10.0 g) and hydrogen gas (40 atm) in an autoclave at 80° C. for 1 h. After being allowed to cool to room temperature, the reaction mixture was filtered through a pad of celite and concentrated under reduced pressure to half its volume. The residue was then acidified with concentrated hydrochloric acid (50 mL) and the remainder of the solvent was removed under reduced pressure to afford 4-[4-trifluoromethyl)phenyl]piperidine hydrochloride (59.0 g, 69%) as an off-white solid, m.p. 196-197° C.; R_{f} 0.06 (75% ethyl acetate in methanol).

### Preparation 9

### 4-(5-Chloro-2-methoxyphenyl)piperidine

To a solution of 2-bromo-4-chloroanisole (164 g, 0.74 mol) in absolute THF (1 L) was added 2.7 M BuLi/heptane (280 mL) under stirring in an atmosphere of argon at −80° C. over a period of 1 h. The mixture was stirred for 30 min then was added a solution of N-Boc-4-piperidone (145 g, 0.73 mol) in absolute THF (250 mL) at −90° C. over a period of 1 h. The temperature was increased to −40° C. during 2 h, and were added 5M NaHSO₄ (160 mL), Na₂SO₄ (300 g) hexane (500 mL), and the mixture was stirred for 10 h. The organic layer was decanted, filtered through silica gel (300 g, 63/100 μm). The residue and silica gel were washed with 40% ethyl acetate/hexane (2×400 mL). The filtrate was evaporated to dryness, the residue was crystallized from a mixture ethyl acetate/hexane to afford tert-butyl 4-(5-Chloro-2-methoxyphenyl)-4-hydroxypiperidine-1-carboxylate in 39% (100 g, 0.29 mol) as white crystals.

To a solution of tert-butyl 4-(5-Chloro-2-methoxyphenyl)-4-hydroxypiperidine-1-carboxylate (90 g, 0.263 mol) in absolute dioxane (200 mL),was added 4 N HCl/dioxane (150 mL, 0.6 mol) under argon. The mixture was stirred for 24 h, evaporated, was added ether, and the evaporation was repeated. To the residue were added water (300 mL) and ether (500 mL). To the obtained mixture ways added Na₂CO₃ (32 g, 0.3 mol) under vigorous stirring, then was added CbzCl (43 mL, 0.3 mol) dropwise under cooling with an ice bath. The bath was removed, and the mixture was stirred for 1 h more. The layers were separated, the aqueous one was extracted with ether (2×200 mL). The combined organic layers were washed with water (200 mL), brine (200 mL), dried with Na₂SO₄, filtered through silica gel (100 g, 40/63 μm), and evaporated. Then was added absolute dioxane, and the evaporation was repeated. To a solution of the residue in absolute dichloromethane (300 mL) ware added Et₃SiH (132 mL, 0.828 mol), and TFA (96 mL, 1.24 mol) under argon. The mixture was stirred for 20 h and evaporated. To the residue were added a saturated K₂CO₃ solution to pH 10′ water (˜200 mL), and the mixture was extracted with ether. The organic fractions were washed with water (2×200 mL), brine (200 mL), dried with Na₂SO₄, filtered through silica gel (100 g, 40/63 μm), and evaporated. To the residue was added absolute dioxane, and the evaporation was repeated. To a solution of the residue in absolute THF (300 mL) was added 1 M BH₃/THF (260 mL) under cooling with an ice bath in an atmosphere of argon. The mixture was stirred at room temperature for 2 h, then was added AcOH (260 mL) under cooling with an ice bath in argon. The mixture was stirred for 24 h, evaporated, to the residue were added a saturated K₂CO₃ solution to pH 10, water (˜200 mL), and the mixture was extracted with ether. The organic fractions were washed with water (2×200 mL), brine (200 mL), dried with Na₂SO₄, and evaporated. The residue was purified on silica gel (500 g, 60/100 μm) with gradient elution from CCl₄, to CCl₄/EtOAc (10:1) to give benzyl 4-(5-Chloro-2-methoxyphenyl)piperidine-1-carboxylate in 77% (73 g) yield as a yellow oil.

To benzyl 4-(5-Chloro-2-methoxyphenylpiperidine-1-carboxylate (73 g, 0.2 mmol) was added concentrated HCl (200 mL). The mixture was refluxed at stirring for 2 h and evaporated to dryness. To the residue were added water (100 mL) and 10N NaCH (20 mL), the mixture was extracted with chloroform (3×200 mL). The organic layers were washed with water (200 mL), brine (200 mL), dried with Na₂SO₄, filtered through silica gel (100 g, 40/63 μm), and evaporated to afford 4-(5-Chloro-2-methoxyphenyl)piperidine in 89% (40 g) yield as white crystals.

The following 4-substituted piperidines were prepared through similar procedure to that described above featuring an addition of an organolithium or Grignard species to N-protected piperidin-4-one:

TABLE 3

| NAME | m/z |
|---|---|
| 3,3-Dimethyl-4-(4-(trifluoromethyl)phenyl)piperidine | 258 |
| 4-(5-Chloro-2-fluorophenyl)piperidine hydrochloride | 214 |
| 4-(2-Fluorophenyl)piperidine hydrochloride | 180.2 |
| 4-(3-methoxyphenyl)piperidine hydrochloride | 192 |
| 4-(3-trifluoromethyl)piperidine hydrochloride | 230 |
| 4-(3,5-difluorophenyl)piperidine hydrochloride | 198 |
| 4-(2-Methoxy-4-trifluoromethylphenyl)piperidine | 260 |
| hydrochloride | |
| 4-(2-Fluoro-4-trifluoromethylphenyl)piperidine hydrochloride | 248 |
| 4-(2-trifluoromethylphenyl)piperidine hydrochloride | 230 |
| 4-p-tolylpiperidine | 176 |
| 4-(2-Fluoro-5-trifluoromethylphenyl)piperidine hydrochloride | 248 |

### Method D (Scheme V)

### Suzuki Coupling/Pyridine Hydrogenation

### Preparation 10

### 4-(2-Methoxyphenyl)piperidine hydrochloride salt

4-Pyridyl boronic acid (2.0 go 16.3 mmol), 2% bromoanisole (2.0 g, 16.3 mmol) and tetrakis(triphenylphosphine) palladium (0) (2.0 g, 16.3 mm ol) were combined in 100 mL of DME and 33 mL of H₂O under N₂ at room temperature. The reaction mixture was then heated to reflux at 85° C. for 17 hours. After cooling to room temperature, the mixture was partitioned between 300 mL brine and 300 mL ethyl acetate. The organic layer was separated and dried over anhydrous Na₂SO₄, filtered and the solvent was evaporated under vacuum. The residue was purified by flash column with 1:1 EtOAc:Hexane to give 865 mg of 4-(2-methoxyphenyl)pyridine as a colorless oil which crystallized under high vacuum. 400 MHz ¹H NMR (CDCl₃) δ (ppm) 8.6 (m, 2H), 7.5 (m, 2H), 7.3-7.4 (m, 2H), 7.0-4.1 (m, 2H), 3.3 (s, 3H); MS (M+1) 186.1. The product was converted to the HCl salt by dissolving the residue in EtOAc and adding 10 mL of 1 M HCl in diethyl ether. The solvent was removed in vacuo to 1.0 g of an off-white solid after drying under high vacuum.

4-(2-Methoxyphenyl)pyridine hydrochloride salt (1.0 g), was dissolved in methanol (23 mL) and platinum (IV) oxide (499 mg) was added. The mixture was then shaked on a Parr shaker under hydrogen (40 psi) for 90 minutes. Additional 500 mg of platinum (IV) oxide was added and the mixture was again placed on the Parr shaker for additional 2 hours. The reaction mixture was then filtered through a pad of celite and the cake was rinsed several times with CH₃OH. The filtrate was evaporated in vacuo to give 1.0 g of 4-(2-methoxyphenyl)piperidine HCl salt as a white solid. 400 MHz ¹H NMR(CDCl₃) δ 9.5-9.7 (broad d, 2H), 7.2 (m, 2H), 6.9 (m, 1H), 6.8-6.9 (d, 1H), 3.8 (s, 3H) 3.6 (d, 2H), 3.1-3.2 (m, 1H), 3.0 (q, 2H), 2.1-2.2 (m, 2H), 2.0 (d, 2H); MS (m/z) 192.0.

### Method E (Scheme V)

### Suzuki Coupling/Pyridine Hydrogenation

### Preparation 11

### Cis-4-(2-Methoxy-4-(trifluoromethylphenyl)-3-methylpiperidine hydrochloride

To a stirred solution of 1-methoxy-3-(trifluoromethyl)benzene (9.8 mL, 68 mmol) in 50 mL THF under N₂ at 0° C. was added n-BuLi (1.6 M in hexanes, 45 mL, 68 mmol) dropwise. The reaction mixture was stirred at 0° C. for 2 h, then triisopropylborate (11.6 mL, 68 mmol) was added. The reaction mixture was slowly warmed up to room temperature and stirred overnight. A solution of 10% HCl in water was added and the mixture was stirred for 1 h. The mixture was extracted with CH₂Cl₂ (3×). The organic layers were combined, washed with brine and dried over Na₂SO₄ to give 8.14 g of 2-methoxy-4-(trifluoromethyl)phenylboronic acid as a viscous oil. The crude was directly used in the next step without further purification.

2-Methoxy-4-(trifluoromethyl)phenylboronic acid (8.14 g, 37 mmol), 4-bromo-3-methylpyridine HCl salt (1.3 g, 5.81 mmol) NaHCO₃ (6.0 g, 70 mmol) and tetrakis (triphenylphosphine) palladium (0) (671 mg, 0.58 mmol) were combined in 9 mL of DME and 9 mL of H₂O under N₂ at room temperature. The mixture was stirred for 10 min and then heated to reflux overnight. After cooling to room temperature, the mixture was partitioned between brine and ethyl acetate. The organic layer was separated and dried over anhydrous Na₂SO₄, filtered and the solvent was evaporated under vacuum. The residue was purified by flash column with 10% EtOAc in hexane to give 1.89 g of 4-(2-methoxy-4-(trifluoromethyl)phenyl)-3-methylpyridine. 400 MHz ¹H NMR(CDCl₃) δ (ppm) 8.5 (s, 1H), 8.45 (d, 1H), 7.30 (d, 1H), 7.22 (t, 1H), 7.17 (s, 1H), 7.06 (d, 1H), 3.8 (s, 3H), 2.1 (s, 3H), MS (M+1) 268. The product as converted to the HCl salt by dissolving the residue in CH₂Cl₂ and adding 2 mL of 4N HCl in dioxane. The solvent was removed in vacuo and the residue was triturated with diethyl ether to give 2.0 g of a pure white solid after filtration.

4-(2-Methoxy-4-(trifluoromethyl)phenyl))-methylpyridine hydrochloride salt (692 mg) was dissolved in ethanol (40 mL) and platinum (IV) oxide (70 mg) was added. The mixture was then shaked on a Parr shaker under hydrogen (40 psi) at 70° C. for 48 h. The reaction mixture was then filtered through a pad of celite and the cake was rinsed several times with ethanol. The filtrate was evaporated in vacuo and co-evaporated with diethyl ether (2×) to give 690 mg of Cis-4-(2-Methoxy-4-(trifluoromethyl)phenyl)-3-methylpiperidine HCl salt as a white solid. 400 MHz ¹H NMR (CD₃OD) δ (ppm) 7.30 (m, 2H), 7.20 (s, 1H). 3.9 (s, 3H), 3.4 G 3.60 (m, 2H), 3.20-3.29 (m, 2H), 3.1-3.18 (m, 1H), 2.55 (m, 1H), 2.40 (m, 1H), 1.76 (m, 1H). 0.80 (d, 3H), MS (m/z) 274, 315 (+CH₃CN). The following substituted piperidines were prepared as above starting with the reaction of substituted/unsubstituted bromopyridines and the appropriate arylboronic acids

TABLE 4

| NAME | m/z |
|---|---|
| 4-(2-methyl-4-(trifluoromethoxy)phenyl)piperidine hydrochloride | 260.2 |
| 4-(2-methyl-4-(trifluoromethyl)phenyl)piperidine hydrochloride | 244.2 |
| 4-(2-Chloro-4-(trifluoromethoxy)phenyl)piperidine hydrochloride | 280.1 |
| 4-(2-Chloro-4-(difluoromethyl)phenyl)piperidine hydrochloride | 262.1 |
| 4-(2-Fluoro-4-isopropoxyphenyl)piperidine hydrochloride | 238.2 |
| 4-(2-Fluoro-4-(difluoromethyl)phenyl)piperidine hydrochloride | 246.2 |
| 4-(4-(Difluoromethyl)phenyl)piperidine hydrochloride | 228.2 |
| 4-(2-Chloro-4-isopropoxyphenyl)piperidine hydrochloride | 254.2 |
| 4-(4-Methoxy-2-(trifluoromethyl)phenyl)piperidine hydrochloride | 260.2 |
| 4-(2-Chloro-4-(trifluoromethyl)phenyl)piperidine hydrochloride | 264.1 |
| 4-(3-Methyl-4-(trifluoromethyl)phenyl)piperidine hydrochloride | 244.2 |
| 4-(3-Methoxy-4-(trifluoromethyl)phenyl)piperidine hydrochloride | 260.2 |
| 4-(2-Methoxy-4-(trifluoromethoxy)phenyl)piperidine hydrochloride | 276.2 |
| 4-(3-Methyl-4-(trifluoromethoxy)phenyl)piperidine hydrochloride | 260.2 |
| 4-(2,4-bis(Trifluoromethyl)phenyl)piperidine hydrochloride | 298.2 |
| 4-(4-Fluoro-2-(trifluoromethyl)phenyl)piperidine hydrochloride | 248.2 |
| 4-(4-Chloro-2-(trifluoromethyl)phenyl)piperidine hydrochloride | 264.1 |
| 4-(2,4-Dichlorophenyl)piperidine hydrochloride | 230.1 |
| 4-(3-Fluoro-4-(trifluoromethyl)phenyl)piperidine hydrochloride | 248.2 |
| Cis-4-(4-Chloro-2-fluoro)phenyl)-3-methylpiperidine hydrochloride | 228.1 |
| Cis-4-(3-Chloro-4-fluoro)phenyl)-3-methylpiperidine hydrochloride | 228.1 |
| Cis-4-(4-trifluormethylphenyl)-3-methylpiperidine hydrochloride | 244.2 |
| Cis-4-(4-trifluoromethylphenyl)-2-methylpiperidine hydrochloride | 244.2 |
| Cis-4-(4-ethoxyphenyl)-3-methylpiperidine hydrochloride | 220.2 |
| Cis-4-(3,4-difluoro)phenyl)-3-methylpiperidine hydrochloride | 212.1 |
| Cis-4-(2-Chloro-4-fluoro)phenyl)-3-methylpiperidine hydrochloride | 228.1 |
| Cis-4-(2-Fluorophenyl)-3-methylpiperidine hydrochloride | 194.2 |
| Cis-4-(4-Fluoro-2-methoxy)phenyl)-3-methylpiperidine | 224.2 |
| hydrochloride | |
| Cis-4-(2,4-difluorophenyl)-3-methylpiperidine hydrochloride | 212.1 |
| Cis-4-(2-Fluoro-4-trifluoromethyl)phenyl)-3-methylpiperidine | 262.1 |
| hydrochloride | |
| Cis-4-(2-Fluoro-4-trifluoromethoxy) phenyl)-3-methylpiperidine | 278.2 |
| hydrochloride | |
| Cis-4-(2-methoxyphenyl)-3-methylpiperidine hydrochloride | 206.2 |
| Cis-4-(3-Fluoro-4-(trifluoromethyl)phenyl)-3-methylpiperidine | 262.2 |
| hydrochloride | |
| Cis-4-(2,4-bis(trifluoromethyl)phenyl)-3-methylpiperidine | 312.2 |
| hydrochloride | |
| Cis-4-(2-Chloro-4-(trifluoromethyl)phenyl)-3-methylpiperidine | 278.1 |
| hydrochloride | |
| Cis-4-(2,4-Dichlorophenyl)-3-methylpiperidine hydrochloride | 244.1 |
| Cis-4-(4-trifluoromethoxypheny)-3-methylpiperidine hydrochloride | 260.2 |
| Cis-4-(4-Chloro-2-methylphenyl)-3-methylpiperidine hydrochloride | 224.2 |
| Cis-3-Methyl-4-(2-methyl-4-(trifluoromethoxy)phenyl)piperidine | 274.2 |
| hydrochloride | |
| Cis-4-(2-Fluoro-4-isopropoxyphenyl)-3-methylpiperidine | 252.2 |
| hydrochloride | |
| Cis-4-(2-Chloro-4-isopropoxyphenyl)-3-methylpiperidine | 268.2 |
| hydrochloride | |
| Cis-4-(4-Methoxy-2-(trifluoromethyl)phenyl)-3-methylpiperidine | 274.2 |
| hydrochloride | |
| Cis-4-(4-(Difluoromethoxy)phenyl)-3-methylpiperidine | 242.2 |
| hydrochloride | |
| Cis-3-Methyl-4-(2,4,5-trifluorophenyl)piperidine hydrochloride | 230.2 |
| Cis-4-(3-Fluoro-4-isopropoxphenyl)-3-methylpiperidine | 252.2 |
| hydrochloride | |
| Cis-4-(2-Chloro-4-(trifluoromethoxy)phenyl)-3-methylpiperidine | 294.1 |
| hydrochloride | |
| Cis-3-Methyl-4-(5,6,7,8-tetrahydronaphthalen-2-yl)piperidine | 230.2 |
| hydrochloride | |
| Cis-4-(4-Fluoro-2-isopropoxyphenyl)-3-methylpiperidine | 252.2 |
| hydrochloride | |
| Cis-4-(3-Chloro-4-isopropoxyphenyl)-3-methylpiperidine | 268.2 |
| hydrochloride | |
| Cis-4-(4-Ethoxy-3-fluorophenyl)-3-methylpiperidine hydrochloride | 238.2 |
| Cis-4-(4-fluoro-3-(trifluoromethyl)phenyl)-3-methylpiperidine | 262.2 |
| hydrochloride | |
| Cis-4-(4-(Difluoromethoxy)-2-fluorophenyl)-3-methylpiperidine | 260.2 |
| hydrochloride | |
| Cis-4-(2,3-Dihydrobenzofuran-5-yl)-3-methylpiperidine | 218.2 |
| hydrochloride | |
| Cis-4-(2-Chloro-4-(difluoromethoxy)phenyl)-3-methylpiperidine | 276.1 |
| hydrochloride | |
| Cis-4-(4-Fluoro-2-(trifluoromethyl)phenyl)-3-methylpiperidine | 262.2 |
| hydrochloride | |
| Cis-4-(4-Fluoro-2-methylphenyl)-3-methylpiperidine hydrochloride | 208.2 |
| Cis-4-(4-Chloro-2-(trifluoromethyl)phenyl)-3-methylpiperidine | 278.1 |
| hydrochloride | |
| Cis-4-(2-Chloro-4-methylphenyl)-3-methylpiperidine hydrochloride | 224.2 |
| Cis-3-Methoxy-4-(4-(trifluoromethyl)phenyl)piperidine | 260.2 |
| hydrochloride | |
| Cis-4-(3-Chloro-4-(trifluoromethoxy)phenyl)-3-methylpiperidine | 294.1 |
| hydrochloride | |
| Cis-4-(2-Methoxy-4-(trifluoromethoxy)phenyl)-3-methylpiperidine | 290.2 |
| hydrochloride | |
| Cis-4-(4-isopropoxy-2-(trifluoromethyl)phenyl)-3-methylpiperidine | 302.2 |
| hydrochloride | |
| Cis-4-(3-Chloro-4-(trifluoromethyl)phenyl)-3-methylpiperidine | 278.2 |
| hydrochloride | |
| Cis-3-methyl-4-(2-methyl-4-(trifluoromethyl)phenyl)piperidine | 258.2 |
| hydrochloride | |
| Cis-3-methyl-4-(2,3,4-trifluorophenyl)piperidine hydrochloride | 230.2 |
| Cis-4-(4-(difluoromethoxy)-2-fluorophenyl)-3-methylpiperidine | 260.2 |
| hydrochloride | |
| Cis-4-(2-(difluoromethoxy)-4-fluorophenyl)-3-methylpiperidine | 260.2 |
| hydrochloride | |

### Method F (Scheme VII),

### Fluorination of Piperidine Ring

### Preparation 12

### 4-(4-fluorophenyl)-4-fluoro-1-piperidine hydrochloride

A solution of [bis(2-methoxyethyl)amino]sulfur trifluoride (BAST) (0.475 ml, 2.6 mmol) in 20 ml of methylene chloride was cooled to −78° C. and a solution of tertbutyl 4-(4-fluorophenyl)-4-hydroxy-1-piperidinecarboxylate (760 mg, 2.6 mmol) (J. Med. Chem. 1992, 35 (22), 4020-26 or Bioorg. Med. Chem. Lett. 2003, 13 (22), 3951-4) in 10 ml of methylene chloride was added dropwise over 5 min. After stirring for 1 h, the mixture was warmed to room temperature, poured into saturated aqueous bicarbonate, and extracted 3 times with methylene chloride. The combined organics were washed with brine, dried over sodium sulfate and concentrated under reduce pressure to provide 700 mg of tert-butyl 4-(4-fluorophenyl)-4-fluoro-1-piperidinecarboxylate as a yellow oil. MS m/z 298.2

A solution of tert-butyl 4-(4-fluorophenyl)-4-fluoro-1-piperidinecarboxylate (0.7 g) in 4M HCl/dioxane (15 ml) was stirred at room temperature. After 2 h the mixture was concentrated under reduced pressure to give 0.55 g of 4-(4-fluorophenyl)-4-fluoro-1-piperidine hydrochloride as an off-white solid. MS m/z 198.2.

The following 4-fluoro-4-aryl piperidines were prepared as above starting with the reaction of BAST and the appropriate tert-butyl 4-aryl-4-hydroxy-1-piperidinecarboxylate:

TABLE 5

| NAME | m/z |
|---|---|
| 4-fluoro-4-(4-chlorophenyl)-1-piperidine hydrochloride | 214.2 |
| 4-fluoro-4-(4-trifluoromethylphenyl)-1-piperidine hydrochloride | 248.2 |
| 4-fluoro-4-(2-methylphenyl)-1-piperidine hydrochloride | |

### Method G (Scheme-VII)

### Preparation 13

### 3-Hydroxy-4-(4-(trifluoromethyl)phenyl)piperidine hydrochloride

To a stirred solution of borane-methylsulfide complex (0.1 mL) in THF (5 mL) under N₂ at 0° C. as added tert-butyl 4-(4-trifluoromethyl)phenyl)-5,6-dihydropyridine-1(2H)-carboxylate (prepared according to preparation 13 using 4-(trifluoromethylphenylboronic acid) (300 mg, 0.92 mmol) in THF (2 mL) dropwise. After the addition was complete, the reaction mixture was stirred at room temperature overnight, then cooled to 0° C. and sodium hydroxide (1N in water, 2 mL) was added dropwise, followed by hydrogen peroxide (2 mL). The resulting mixture was heated to 60° C. for 45 min, then cooled to room temperature and diluted with 20 mL CH₂Cl₂. The mixture was washed with water, brine and dried over Na₂SO₄. The solvent was removed in vacuo to give 278 mg of tert-butyl 3-hydroxy-4-(4-(trifluoromethyl)phenyl)piperidine-1-carboxylate as a mixture of diastereomers. 3-Hydroxy-4-(4-trifluoromethyl)phenyl)piperidine-1-carboxylate (230 mg) was dissolved in CH₂Cl₂ (1.5 mL) and 0.2 mL of 4N HCl in dioxane was added. The mixture was stirred at room temperature overnight and the solvent was removed under reduced pressure to give 198 mg of 3-hydroxy-4-(4-(trifluoromethyl)phenyl)piperidine hydrochloride. MS m/z 246.2.

### Preparation 14

### 3-Fluoro-4-(4-(trifluoromethyl)phenyl)piperidine hydrochloride

A solution of [bis(2-methoxyethyl)amino]sulfur trifluoride (BAST) (77 uL, 0.76 mmol) in 1.5 ml of methylene chloride was cooled to −78° C. and a solution of tert-butyl 3-hydroxy-4-(4-(trifluoromethyl)phenyl)piperidine-1-carboxylate (250 mg, 0.72 mmol) in 1 mL of methylene chloride was added dropwise over 5 min. After stirring for 1 h, the mixture was warmed to room temperature, poured into saturated aqueous bicarbonate, and extracted 3 times with methylene chloride. The combined organics were washed with brine, dried over sodium sulfate and concentrated under reduce pressure to provide 259 mg of tert-butyl 3-fluoro-4-(4-(trifluoromethyl)phenyl)piperidine-1-carboxylate as a yellow oil. The residue was dissolved in CH₂Cl₂ (1.5 mL) and 0.2 mL of 4N HCl in dioxane was added. The mixture was stirred at room temperature overnight and the solvent was removed under reduced pressure to give 214 mg of 3-fluoro-4-(4-(trifluoromethyl)phenyl)piperidine hydrochloride. MS m/z 248.2.

The references for the amines that are known in the literature are listed in Table 6.

TABLE 6

| IUPAC Name | References |
|---|---|
| 4-(3-trifluoromethyl-phenyl)-piperidin-4-ol | Collect. Czech. Chem. Commun., 1973, 38, 3829-3901 |
| 3H-Spiro[2-benzofuran-1,4′-piperidine] | J. Med. Chem., 1976, 19, 1315-1324 |
| Spiro[indene-1,4′-piperidine] | J. Med. Chem. 1992, 35, 3919-3927 |
| 1-(4-phenyl-piperidin-4-yl)-ethanone | J. Med. Chem. 1970, 13, 644-648 |
| 4-(4-chloro-3-trifluoromethyl-phenyl)- | Collect. Czech. Chem. Commun. 1973, 38, 3879-3901 |
| piperidin-4-ol | |
| 1-(4-phenyl-piperidin-4-yl)-propan-1-one | J. Chem. Soc. 1959, 1143-1147 |
| 4-phenyl-piperidine | Helv. Chim. Acta 1973, 56, 2348-2377 |
| 4-(4-bromo-phenyl)-piperidin-4-ol | J. Med. Chem. 1999, 42, 4680-4694 |
| 4-(4-chloro-phenyl)-piperidin-4-ol | J. Med. Chem. 1970, 13, 644-648 |
| 4-phenyl-piperidine-4-carbonitrile | J. Med. Chem. 1970, 13, 644-648 |
| 4-(2-isopropoxy-phenyl)-piperidine | J. Med. Chem. 1998, 41, 1997-2009 |
| 3-(3,4-dimethyl-piperidin-4-yl)-benzoic | Bioorg. Med. Chem. Lett. 2003, 13, 4459-4462 |
| acid methyl ester | |
| 3,5-dimethyl-4-phenyl-piperidin-4-ol | J. Med. Chem. 1964, 7, 726-728 |
| 1-methylspiro[indoline-1,4′-piperidine] | J. Med. Chem. 1997, 3905-3914 |
| 3-phenyl-pyrrolidine | J. Med. Chem. 1971, 14, 737-742 |
| 3-(4-fluoro-phenyl)-pyrrolidine | Bioorg. Med. Chem. Lett. 1999, 9, 1379-1384 |
| 3-benzyl-pyrrolidine | Med. Chem. Res. 1997, 7, 76-86 |
| 4-(2-chloro-phenyl)-piperidine | 1960, U.S. Pat. No. 2891066 |
| 4-(2-trifluoromethyl-phenyl)-piperidine | J. Med. Chem. 2005, 1857-1872 |
| Spiro[1-benzofuran-3,4′-piperidine] | J. Med. Chem. 1995, 38, 2009-2017 |
| 2-piperidin-4-yl-phenol | Chem. Pharm. Bull. 2000, 48, 1978-1985 |
| 5-phenyl-azepan-2-one | Chem. Pharm. Bull. 1969, 17, 434-453 |
| 4-(3,4-dimethyl-phenyl)-piperidine | Bioorg. Med. Chem. Lett. 1998, 8, 1499-1502 |
| 4-m-tolyl-piperidine | Chem. Pharm. Bull. 1987, 35, 2825-2839 |
| 3-(benzo[1,3]dioxol-5-yloxymethyl)-4-(4- | J. Med. Chem. 1997, 40, 1049-1062 |
| fluoro-phenyl)-piperidine | |
| 2-piperidin-4-yl-benzonitrile | Bioorg. Med. Chem. Lett. 2000, 10, 1917-1920 |
| 4-(3-chloro-phenyl)-piperidine | Eur. J. Med. Chem. Chim. Ther. 1987, 22, 337-346 |
| 3-piperidin-4-yl-benzonitrile | Bioorg. Med. Chem. 2005, 13, 2859-2872 |
| 4-(4-chloro-phenyl)-piperidine | Arzneim. Forsch. 1967, 17, 1145-1149 |
| 4-(4-methoxy-phenyl)-piperidine | NL 6510107 (1966) |
| 4-(4-ethyl-phenyl)-piperidine | DE 2801195 (1978) |
| 4-o-tolyl-piperidine | Bioorg. Med. Chem. Lett. 1998, 8, 1851-1856 |
| 4-(3-fluoro-phenyl)-piperidine | Chem. Pharm. Bull. 1987, 35, 2825-2839 |
| 4-p-tolyl-piperidine | Collect. Czech. Chem. Commun. 1975, 40, 3904-3923 |
| (R)-3-benzyl-pyrrolidine | Tetrahedron Lett. 1994, 35, 973-976 |
| (S)-3-benzyl-pyrrolidine | Tetrahedron Lett. 1994, 35, 973-976 |
| (R)-3-phenyl-pyrrolidine | Acta Chem. Scand. 1990, 44, 42-49 |
| (S)-3-phenyl-pyrrolidine | Synthesis 1991, 1023-1026 |
| (S)-5-phenyl-azepan-2-one | J. Am. Chem. Soc. 1990, 112, 4879-4891 |

### Preparation of Compounds of Formula (I)

For each method, a general procedure or a representative synthesis is described. Other examples prepared via similar method are listed in table 8 with method number indicated.

### Method H

0.25 M stock solutions of amines (II) and aldehydes (III) in DOE were prepared. When applicable, the aldehyde salt forms were neutralized by addition of 4 equivalents of DIPEA. A 0.25 M fine suspension of NaBH(OAc)₃ in anhydrous DMF/DCE mixture (20/80) was prepared. To each vial was added 0.2 mL of a solution of amine (II) followed by 0.2 mL of a solution of aldehyde (III) and 0.5 mL of the NaBH(OAc)₃ suspension to each vial. The vials were capped and shaken at room temperature for 16 h. Additional 0.5 mL of the NaBH(OAc)₃ suspension was added to each vial the vials were vortexed, capped, and shaken at room temperature for 16 h. The solvent was removed under reduced pressure. 1 mL of DMSO and 0.1 mL of water were added to each vial. The samples were vortexed for 1 h. 0.05 mL of concentrated NH₄OH was added to each vial. The samples were filtered and directly submitted to HPLC purification.

### Method I

### Example 1

### 2-((4-(2-Methoxy-4-(trifluoromethyl)phenyl)piperidin-1-yl)methyl)-1-methyl-1H-imidazo[4,5-b]pyridine

To a stirred solution of 4-(2-Methoxy-4-(trifluoromethyl)phenyl)piperidine hydrochloride salt (444 mg, 1.5 mmol) in CH₂Cl₂ under N₂ at room temperature was added triethylamine (1.7 mL, 12.0 mmol), MgSO₄ (20 mg) and 1-Methyl-1H-imidazo[4,5-b]pyridine-2-carbaldehyde Hydrochloride Hydrate (323 mg, 1.5 mmol). The reaction mixture was stirred for 30 min, then NaBH(OAc)₃ (477 mg, 2.25 mmol) was added. The mixture was stirred at room temperature overnight and was then diluted with methylene chloride (50 mL) and washed with water, brine and dried with Na₂SO₄. The solvent was removed in vacuo and the residue was purified by flash column with 1-10% MeOH in CH₂CO₂ to give 409 mg of 2-((4-(2-methoxy-4-(trifluoromethyl)phenyl)piperidin-1-yl)methyl)-1-methyl-1H-imidazo[4,5-b]pyridine as a light tan foam. The residue was dissolved in MeOH (3 mL) and a 4N HCl solution in dioxane (0.3 mL) was added and the mixture was stirred for 10 min. The solvent was removed in vacuo to give 433 mg of 2-((4-(2-methoxy-4-(trifluoromethyl)phenoxy)piperidin-1-yl)methyl)-1H-imidazo[4,5-b]pyridine hydrochloride salt as a tan solid. 400 MHz ¹H NMR (CD3OD) δ (ppm) 8.57 (d, 1H), 8.32 (d, 1H), 7.56 (m, 1H), 7.43 (d, 1H), 7.28 (d, 1H), 7.23 (s, 1H), 4.94 (s, 2H). 4.03 (m, 2H), 4.00 (s, 3H), 3.93 (s, 3H): 3.40-3.50 (m, 3H), 2.10-2.25 (m, 4H); MS (m/z) 405.2.

### Method J (Scheme VI)

Stock solutions of amines (XXIX) (0.15 M in THF), PPh₃ (0.5 M in THF), and di-t-butylazadicarboxylate (0.3 M in THF) were prepared. The vials containing alcohols of formula (XXX) was added 1.2 mL of THF and the mixtures was sonicated. To each vial was added 0.667 mL of the solution of amine (XXIX). 0.50 mL of the PPh₃ solution, and 0.667 mL of the di-t-butylazodicarboxylate solution. The vials were capped and shaken at room temperature for 16 h. The solvent was evaporated under the reduced pressure and the residues were dissolved in 1 mL of MeOH. The obtained solutions were loaded onto Waters Oasis MCX cartridges (6 cc/500 mg) previously conditioned with 2 mL of MeOH. The vials were rinsed with 1 mL of methanol and the obtained solutions were loaded on the cartridges as well. The cartridges were eluted using 4.5 mL of 1 M NH₃ in MeOH into collection vials and the solvents were removed under nitrogen at 35° C.

Stock solutions of aldehyde (III) (0.25 M in DCE), and NaBH(OAc)₃ (0.25 M in CHCl₃) were prepared. The residue in each reaction vial was dissolved in 0.6 mL of DCE. To each vial was added 0.4 mL of the solution of aldehyde (III) and 1.2 mL of NaBH(OAc)₃ solution. The vials were capped and shaken at room temperature for 16 h. 2 ml of 10% aqueous NH₄OH was added to each vial and the mixtures were vortexed thoroughly. The mixtures were loaded onto Varian ChemElut cartridges and eluted with DCE (2×3 mL) into collection vials. The solvents were removed under nitrogen at 35° C. The residues were dissolved in 1 mL of DMSO, filtered and submitted to HPLC purification.

### Method K [Chiral Separation]

### Example 2

### 2-((Cis-4-(4-chloro-2-fluorophenyl)-3-methylpiperidin-1-yl)methyl)-1-methyl-1H-imidazo[4,5-b]pyridine

To A stirred solution of Cis-4-(4-chloro-2-fluorophenyl)-3-methylpiperidine hydrochloride salt (1.44 g, 4.23 mmol) in CH₂Cl₂ (25 mL) under N₂ at room temperature was added triethylamine (1.3 mL, 12.7 mmol), MgSO₄ (1.5 g) and 1-Methyl-1H-imidazo[4,5-b]pyridine-2-carbaldehyde Hydrochloride Hydrate (951 mg 4.23 mmol). The reaction mixture was stirred at rt for 30 min, then NaBH(OAc)₃ (1.34 g, 6.35 mmol) was added. The mixture was stirred at room temperature overnight. The mixture was then diluted with methylene chloride (150 mL) and washed with water, brine and dried with Na₂SO₄. The solvent was removed in vacuo and the residue was purified by flash column with 1-3% MeOH in CH₂Cl₂ to give 1.48 g of the desired product as a racemic mixture. Enantiomers were separated on a Chiralcel OJ-H column (3 cm×25 cm) with 83/17 CO₂/MeOH as mobile phase at flow rate of 65 g/min. The individual enantiomers were then dissolved in DCM and treated with 4N HCl in dioxane (3 eq). Solvent was removed in vacuo and the resulted solids were triturated with ethylether and dried under vacuum. ¹H-NMR spectra and LC-MS from the two enantiomers are identical. 400 MHz ¹H NMR (CD₃OD) δ (ppm) 8.88 (d, 1H), 8.72 (d, 1H, 7.90 (m, 1H), 7.25 (m, 3H), 5.08 (s, 2H), 4.10 (s, 3H), 4.05 (m, 2H), 3.60 (m, 3H), 2.66 (m, 1H), 2.55 (m, 1H), 2.03 (m, 1H), 1.03 (d, 3H), LC-MS: retention time 2.3 min, MS⁺ (m/z) 373.1:

### Example 2a

### 2-((Cis-4-(4-chloro-2-fluorophenyl)-3-methylpiperidin-1-yl)methyl)-1-methyl-1H-imidazo[4,5-b]pyridine, enantiomer #1: 604.4 mg white solid, Chiral column retention time 11.50 min,

### Example 2b

### 2-((Cis-4-(4-chloro-2-fluorophenyl)-3-methylpiperidin-1-yl)methyl)-1-methyl-1H-imidazo[4,5-b]pyridine, enantiomer #2: 518.2 mg white solid, Chiral column retention time 13.5 min,

### Example 3

### 2-((Cis-4-(2-fluoro-4-(trifluoromethyl)phenyl)-3-methylpiperidin-1-yl)methyl)-1-methyl-1H-imidazo[4,5-c]pyridine

To a stirred solution of Cis-4-(2-fluoro-4-(trifluoromethyl)phenyl)-3-methylpiperidine hydrochloride salt (600 mg, 2.95 mmol) in CH₂Cl₂ (10 mL), under N₂ at room temperature was added triethylamine (0.85 mL, 6.05 mmol), MgSO₄ (600 mg), and 1-Methyl-1H-imidazo[4,5-c]pyridine-2-carbaldehyde hydrochloride hydrate (562 mg, 2.95 mmol). The reaction mixture was stirred at rt for 30 min, then NaBH(OAc)₃ (638 mg, 3 0 mmol) was added. The mixture was stirred at room temperature overnight. The mixture was then diluted with methylene chloride (100 mL), washed with water brine and dried with Na₂SO₄. The solvent was removed in vacuo and the residue was purified by flash column with 1-5%, MeOH in CH₂Cl₂ to give 631 mg of the desired product as a racemic mixture. 400 MHz ¹H NMR(CD₃Cl₃) δ (ppm) 8.99 (s, 1H), 8.42 (d, 1H), 7.24 (m, 2H), 7.20 (m, 2H), 3.91 (s, 3H), 3.81 (5s 2H), 3.19 (m, 1H), 2.94 (m, 2H), 2.71 (dd, 1H), 2.53 (dd, 1H), 2.26 (m, 1H), 2.15 (m, 1H), 1.53 (m, 1H), 0.73 (d, 3H). LC-MS: retention time 2.2 min, MS⁺ (m/z) 407.1. Two enantiomers were separated using a Chiralpak AS column (10 cm×50 cm) with 92/8 Heptane/EtOH as mobile phase at flow rate of 475 mL/min. The individual enantiomers were then dissolved in DCM and treated with 4N HCl in dioxane (3 eq). Solvent was removed in vacuo and the resulted solids were triturated with ethylether and dried under vacuum:

### Example 3a

### 2-((Cis-4-(2-fluoro-4-(trifluoromethyl)phenyl)-3-methylpiperidin-1-yl)methyl)-dimethyl-1H-imidazo[4,5-c]pyridine, enantiomer #1: 268 mg white solid, Chiral column retention time 7.12 min.

### Example 3b

### 2-(Cis-4-(2-fluoro-4-trifluoromethyl)phenyl)-3-methylpiperidin-1-yl)methyl)-1-methyl-1H-imidazo[4,5-c,]pyridine, enantiomer #2: 259 mg white solid, Chiral column retention time 8.96 min.

Additional examples synthesized with method K using the appropriate Cis-4-aryl-3-methyl piperidines and imidazopyridine-2-carbaldehydes are shown in Table 7 with chiral separation conditions and retention times of the two resulting enantiomers described. Additional characterization data and biological data of these compounds and additional examples are included in Tables 8 and 9.

TABLE 7

| | | | | | | Retention | |
|---|---|---|---|---|---|---|---|
| | | | | | | Time | Retention |
| | | | | | | (min): | Time (min): |
| | | | | | | Eantiomer | Eantiomer |
| Ex. | Compound Name | Column | Mobil phase | Flow rate | | # 1 | #2 |
| 4 | 2-((Cis-4-(2-methoxy-4- | Chiralpak | 85/15 | 40 | mL/min | 11.8 | 12.6 |
| | (trifluoromethyl)phenyl)- | AD-H | heptane/EtOH | | | | |
| | 3-methylpiperidin-1- | (3 cm × 25 cm) | | | | | |
| | yl)methyl)-1-methyl-1H- | | | | | | |
| | imidazo[4,5-b]pyridine | | | | | | |
| 5 | 2-((Cis-4-(2-methoxy-4- | Chiralpak | 65/35 | 500 | mL/min | 5.74 | 12.93 |
| | (trifluoromethyl)phenyl)- | AD (10 cm × | heptane/EtOH | | | | |
| | 3-methylpiperidin-1- | 50 cm) | 0.1% DEA | | | | |
| | yl)methyl)-1-methyl-1H- | | | | | | |
| | imidazo[4,5-c]pyridine | | | | | | |
| 6 | 2-((Cis-4-(2-fluoro-4- | Chiralpak | 80/20 | 475 | mL/min | 8.12 | 11.6 |
| | (trifluoromethyl)phenyl)- | AD (10 cm × | Heptane/EtOH | | | | |
| | 3-methylpiperidin-1- | 50 cm) | | | | | |
| | yl)methyl)-1-methyl-1H- | | | | | | |
| | imidazo[4,5-b]pyridine | | | | | | |
| 7 | 2-((Cis-4-(2-methoxy-4- | Chiralpak | 65/35 | 475 | mL/min | 5.92 | 9.82 |
| | (trifluoromethyl)phenyl)- | AD (10 cm × | Heptane/EtOH | | | | |
| | 3-methylpiperidin-1- | 50 cm) | | | | | |
| | yl)methyl)-1-methyl-1H- | | | | | | |
| | imidazo[4,5-c]pyridine | | | | | | |
| 8 | 2-((Cis-4-(2-fluoro-4- | Chiral pak | 85/15 | 500 | mL/min | 9.01 | 11.62 |
| | (trifluoromethoxy)phenyl)- | AD (10 cm × | Heptane/EtOH | | | | |
| | 3-methylpiperidin-1- | 50 cm) | | | | | |
| | yl)methyl)-1-methyl-1H- | | | | | | |
| | imidazo[4,5-b]pyridine | | | | | | |
| 9 | 2-((Cis-4-(3-fluoro-4- | Chiralpak | 80/20 | 20 | mL/min | 9.56 | 13.75 |
| | (trifluoromethyl)phenyl)- | AD (2.1 cm × | heptane/isopropanol | | | | |
| | 3-methylpiperidin-1- | 25 cm) | | | | | |
| | yl)methyl)-1-methyl-1H- | | | | | | |
| | imidazo[4,5-b]pyridine | | | | | | |
| 10 | 2-((Cis-4-(2-fluoro-4- | Chiralpak | 90/10 | 500 | mL/min | 7.05 | 9.31 |
| | (trifluoromethoxy)phenyl)- | AD (10 cm × | Heptane/EtOH | | | | |
| | 3-methylpiperidin-1- | 50 cm) | | | | | |
| | yl)methyl)-1-methyl-1H- | | | | | | |
| | imidazo[4,5-c]pyridine | | | | | | |
| 11 | 2-((Cis-4-(2-chloro-4- | Chiralpak | 90/10 | 475 | mL/min | 17.41 | 19.66 |
| | (trifluoromethyl)phenyl)- | AD (5 cm × | Heptane/EtOH | | | | |
| | 3-methylpiperidin-1- | 50 cm) | | | | | |
| | yl)methyl)-1-methyl-1H- | | | | | | |
| | imidazo[4,5-b]pyridine | | | | | | |
| 12 | 2-((Cis-4-(2-chloro-4- | Chiralpak | 75/25 | 500 | mL/min | 9.00 | 17.02 |
| | (trifluoromethyl)phenyl)- | AD (10 cm × | Heptane/EtOH | | | | |
| | 3-methylpiperidin-1- | 50 cm) | | | | | |
| | yl)methyl)-1-methyl-1H- | | | | | | |
| | imidazo[4,5-c]pyridine | | | | | | |
| 13 | 1-Methyl-2-((Cis-3- | Chiralpak | 75/25 | 500 | mL/min | 8.71 | 10.44 |
| | methyl-4-(4- | AD (10 cm × | Heptane/EtOH | | | | |
| | (trifluoromethoxy)phenyl)- | 50 cm) | | | | | |
| | piperidin-1-yl)methyl)- | | | | | | |
| | 1H-imidazo[4,5- | | | | | | |
| | c]pyridine | | | | | | |
| 14 | 2-((Cis-4-(3-fluoro-4- | Chiralpak | 80/20 | 500 | mL/min | 10.56 | 13.11 |
| | (trifluoromethyl)phenyl)- | AD (10 cm × | Heptane/EtOH | | | | |
| | 3-methylpiperidin-1- | 50 cm) | | | | | |
| | yl)methyl)-1-methyl-1H- | | | | | | |
| | imidazo[4,5-c]pyridine | | | | | | |
| 15 | 2-((Cis-4-(2,4- | Chiralpak | 80/20 | 500 | mL/min | 8.76 | 11.35 |
| | dichlorophenyl)-3- | AD (10 cm × | Heptane/EtOH | | | | |
| | methylpiperidin-1- | 50 cm) | | | | | |
| | yl)methyl)-1-methyl-1H- | | | | | | |
| | imidazo[4,5-b]pyridine | | | | | | |
| 16 | 1-methyl-2-((Cis-3- | Chiralpak | 50/50 | 500 | mL/min | 5.74 | 8.06 |
| | methyl-4-(2,4,5- | AD (10 cm × | Heptane/EtOH | | | | |
| | trifluorophenyl)piperidin- | 50 cm) | | | | | |
| | 1-yl)methyl)-1H- | | | | | | |
| | imidazo[4,5-c]pyridine | | | | | | |
| 17 | 2-((Cis-4-(4- | Chiralpak | 60/40 | 500 | mL/min | 6.00 | 7.87 |
| | (difluoromethoxy)phenyl)- | AD (10 cm × | Heptane/EtOH | | | | |
| | 3-methylpiperidin-1- | 50 cm) | | | | | |
| | yl)methyl)-1-methyl-1H- | | | | | | |
| | imidazo[4,5-b]pyridine | | | | | | |
| 18 | 2-((Cis-4-(2-chloro-4- | Chiralpak | 50/50 | 500 | mL/min | 4.67 | 10.48 |
| | isopropoxyphenyl)-3- | AD (10 cm × | Heptane/EtOH | | | | |
| | methylpiperidin-1- | 50 cm) | | | | | |
| | yl)methyl)-1-methyl-1H- | | | | | | |
| | imidazo[4,5-b]pyridine | | | | | | |
| 19 | 2-((Cis-4-(2-fluoro-4- | Chiralpak | 50/50 | 500 | mL/min | 5.75 | 11.51 |
| | isopropoxyphenyl)-3- | AD (10 cm × | Heptane/EtOH | | | | |
| | methylpiperidin-1- | 50 cm) | | | | | |
| | yl)methyl)-1-methyl-1H- | | | | | | |
| | imidazo[4,5-c]pyridine | | | | | | |
| 20 | 2-((Cis-4-(2-fluoro-4- | Chiralpak | 50/50 | 500 | mL/min | 4.73 | 11.12 |
| | isopropoxyphenyl)-3- | AD (10 cm × | Heptane/EtOH | | | | |
| | methylpiperidin-1- | 50 cm) | | | | | |
| | yl)methyl)-1-methyl-1H- | | | | | | |
| | imidazo[4,5-b]pyridine | | | | | | |
| 21 | 2-((Cis-4-(2,4- | Chiralpak | 90/10 | 500 | mL/min | 7.72 | 9.34 |
| | dichlorophenyl)-3- | AS (10 cm × | Heptane/EtOH | | | | |
| | methylpiperidin-1- | 50 cm) | | | | | |
| | yl)methyl)-1-methyl-1H- | | | | | | |
| | imidazo[4,5-c]pyridine | | | | | | |
| 22 | 2-((Cis-4-(2,4- | Chiralcel | 85/15 | 500 | mL/min | 9.25 | 11.80 |
| | bis(trifluoromethyl)phenyl)- | OJ (10 cm × | Heptane/iPrOH | | | | |
| | 3-methylpiperidin-1- | 50 cm) | | | | | |
| | yl)methyl)-1-methyl-1H- | | | | | | |
| | imidazo[4,5-b]pyridine | | | | | | |
| 23 | 2-((Cis-4-(2-chloro-4- | Chiralcel | 94/6 | 65 | g/min | 6.97 | 7.62 |
| | (trifluoromethoxy)phenyl)- | OJ-H (2.1 cm × | CO₂/MeOH | | | | |
| | 3-methylpiperidin-1- | 25 cm) | | | | | |
| | yl)methyl)-1-methyl-1H- | | | | | | |
| | imidazo[4,5-b]pyridine | | | | | | |
| 24 | 2-((Cis-4-(2-chloro-4- | Chiralpak | 85/15 | 65 | g/min | 6.84 | 8.15 |
| | (trifluoromethoxy)phenyl)- | AD-H (3 cm × | CO₂/MeOH | | | | |
| | 3-methylpiperidin-1- | 25 cm) | | | | | |
| | yl)methyl)-1-methyl-1H- | | | | | | |
| | imidazo[4,5-c]pyridine | | | | | | |
| 25 | 2-((Cis-4-(4-fluoro-2- | Chiralpak | 85/15 | 40 | mL/min | 10.50 | 12.07 |
| | (trifluoromethyl)phenyl)- | AD-H (3 cm × | Heptane/MeOH | | | | |
| | 3-methylpiperidin-1- | 25 cm) | 0.2% | | | | |
| | yl)methyl)-1-methyl-1H- | | DEA | | | | |
| | imidazo[4,5-b]pyridine | | | | | | |
| 26 | 2-((Cis-4-(2-chloro-4- | Chiralpak | 50/50 | 1 | mL/min | 4.67 | 7.00 |
| | (difluoromethoxy)phenyl)- | AD (4.6 cm × | Heptane/EtOH | | | | |
| | 3-methylpiperidin-1- | 25 cm) | 0.2% DEA | | | | |
| | yl)methyl)-1-methyl-1H- | | | | | | |
| | imidazo[4,5-b]pyridine | | | | | | |
| 27 | 2-((Cis-4-(2-chloro-4- | Chiralpak | 50/50 | 1 | mL/min | 6.87 | 8.98 |
| | (difluoromethoxy)phenyl)- | AD (4.6 cm × | Heptane/EtOH | | | | |
| | 3-methylpiperidin-1- | 25 cm) | 0.2% DEA | | | | |
| | yl)methyl)-1-methyl-1H- | | | | | | |
| | imidazo[4,5-c]pyridine | | | | | | |
| 28 | 2-((Cis-4-(2-chloro-4- | Chiralpak | 50/50 | 1 | mL/min | 6.31 | 8.95 |
| | (difluoromethoxy)phenyl)- | AD (4.6 cm × | Heptane/EtOH | | | | |
| | 3-methylpiperidin-1- | 25 cm) | 0.2% DEA | | | | |
| | yl)methyl)-1-methyl-1H- | | | | | | |
| | imidazo[4,5-c]pyridine | | | | | | |
| 29 | 2-((Cis-4-(3-chloro-4- | Chiralpak | 75/25 | 10 | mL/min | 6.38 | 7.02 |
| | isopropoxyphenyl)-3- | AD-H (10 cm × | CO₂/MeOH | | | | |
| | methylpiperidin-1- | 250 cm) | | | | | |
| | yl)methyl)-1-methyl-1H- | | | | | | |
| | imidazo[4,5-b]pyridine | | | | | | |
| 30 | 2-((Cis-4-(4- | Chiralcel | 85/15 | 65 | g/min | 6.41 | 9.33 |
| | (difluoromethoxy)-2- | OD-H (3 cm × | CO₂/MeOH | | | | |
| | fluorophenyl)-3- | 25 cm) | | | | | |
| | methylpiperidin-1- | | | | | | |
| | yl)methyl)-1-methyl-1H- | | | | | | |
| | imidazo[4,5-c]pyridine | | | | | | |
| 31 | 2-((Cis-4-(4- | Chiralpak | 50/50 | 40 | mL/min | 6.72 | 8.59 |
| | (difluoromethoxy)-2- | AD-H (3 cm × | Heptane/EtOH | | | | |
| | chlorophenyl)-3- | 25 cm) | | | | | |
| | methylpiperidin-1- | | | | | | |
| | yl)methyl)-1-methyl-1H- | | | | | | |
| | imidazo[4,5-c]pyridine | | | | | | |
| 32 | 2-((Cis-4-(4- | Chiralpak | 50/50 | 40 | mL/min | 4.65 | 7.18 |
| | (difluoromethoxy)-2- | AD-H (3 cm × | Heptane/EtOH | | | | |
| | chlorophenyl)-3- | 25 cm) | 0.2% DEA | | | | |
| | methylpiperidin-1- | | | | | | |
| | yl)methyl)-1-methyl-1H- | | | | | | |
| | imidazo[4,5-b]pyridine | | | | | | |
| 33 | 2-((Cis-4-(4-fluoro-2- | Chiralpak | 80/20 | 500 | mL/min | 10.61 | 12.99 |
| | methoxyphenyl)-3- | AD (10 cm × | Heptane/EtOH | | | | |
| | methylpiperidin-1- | 50 cm) | | | | | |
| | yl)methyl)-1-methyl-1H- | | | | | | |
| | imidazo[4,5-c]pyridine | | | | | | |
| 34 | 2-((Cis-4-(3-chloro-4- | Chiralpak | 75/25 | 10 | mL/min | 3.99 | 5.34 |
| | (trifluoromethoxy)phenyl)- | AD-H (10 cm × | CO₂/MeOH | | | | |
| | 3-methylpiperidin-1- | 250 cm) | | | | | |
| | yl)methyl)-1-methyl-1H- | | | | | | |
| | imidazo[4,5-c]pyridine | | | | | | |
| 35 | 2-(((3S,4S)-4-(2- | Chiralpak | 90/10 | 40 | mL/min | 12.1 | 14.4 |
| | methoxy-4- | AD-H | Heptane/EtOH | | | | |
| | (trifluoromethoxy)phenyl)- | (3 cm × 25 cm) | | | | | |
| | 3-methylpiperidin-1- | | | | | | |
| | yl)methyl)-1-methyl-1H- | | | | | | |
| | imidazo[4,5-b]pyridine | | | | | | |
| 36 | 2-(((3S,4S)-4-(2- | Chiralpak | 70/30 | 40 | mL/min | 6.24 | 8.91 |
| | methoxy-4- | AD-H | Heptane/EtOH | | | | |
| | (trifluoromethoxy)phenyl)- | (3 cm × 25 cm) | | | | | |
| | 3-methylpiperidin-1- | | | | | | |
| | yl)methyl)-1-methyl-1H- | | | | | | |
| | imidazo[4,5-c]pyridine | | | | | | |
| 37 | 2-((Cis-4-(4-chloro-2- | Chiralpak | 70/30 | 40 | mL/min | 7.56 | 13.64 |
| | (trifluoromethyl)phenyl)- | AD-H | Heptane/EtOH | | | | |
| | 3-methylpiperidin-1- | (3 cm × 25 cm) | | | | | |
| | yl)methyl)-1-methyl-1H- | | | | | | |
| | imidazo[4,5-c]pyridine | | | | | | |

Enantiomer 1 is designated to the material eluted from column with shorter retention time and enantiomer 2 is designated to the material eluted from column with longer retention time.

TABLE 8
Examples with Data
The following specific compounds were prepared following the similar procedures to
the preparations and examples described above, using the appropriate intermediates and
reagents.

| | | EC50 | Molecular | Caculated | Observed | Retention | |
|---|---|---|---|---|---|---|---|
| Entry # | Compound Name | (uM) | Formular | Mass | Mass | time | Procedure |
| 1 | 2-({4-[2-(2- | 0.409 | C24H30N4O | 390.24 | 390.2413 | 1.09 | J |
| | cyclopropylethoxy)phenyl]- | | | | | | |
| | piperidin-1- | | | | | | |
| | yl}methyl)-3-methyl-3H- | | | | | | |
| | imidazo[4,5-b]pyridine | | | | | | |
| 2 | 2-{[4-(2- | 1.89 | C23H30N4O | 378.24 | 378.2413 | 1.08 | J |
| | isobutoxyphenyl)piperidin- | | | | | | |
| | 1-yl]methyl}-3- | | | | | | |
| | methyl-3H-imidazo[4,5- | | | | | | |
| | b]pyridine | | | | | | |
| 3 | 3-methyl-2-[(4-{2-[(1- | 4.72 | C24H30N4O | 390.24 | 390.2413 | 1.08 | J |
| | methylcyclopropyl)methoxy]- | | | | | | |
| | phenyl}piperidin- | | | | | | |
| | 1-yl)methyl]-3H- | | | | | | |
| | imidazo[4,5-b]pyridine | | | | | | |
| 4 | 3-methyl-2-({4-[2-(prop- | 0.447 | C22H24N4O | 360.2 | 360.1945 | 0.93 | J |
| | 2-yn-1- | | | | | | |
| | yloxy)phenyl]piperidin- | | | | | | |
| | 1-yl}methyl)-3H- | | | | | | |
| | imidazo[4,5-b]pyridine | | | | | | |
| 5 | 3-methyl-2-({4-[3- | 0.54 | C20H21F3N4 | 374.17 | 375.172 | 2.17 | H |
| | (trifluoromethyl)phenyl]- | | | | | | |
| | piperidin-1-yl}methyl)- | | | | | | |
| | 3H-imidazo[4,5- | | | | | | |
| | b]pyridine | | | | | | |
| 6 | 2-{[4-(2,5- | 2.18 | C21H26N4O2 | 366.21 | 367.206 | 1.82 | H |
| | dimethoxyphenyl)piperidin- | | | | | | |
| | 1-yl]methyl}-3- | | | | | | |
| | methyl-3H-imidazo[4,5- | | | | | | |
| | b]pyridine | | | | | | |
| 7 | 3-methyl-2-({4-[4- | 0.285 | C20H21F3N4 | 374.17 | 375.172 | 2.2 | H |
| | (trifluoromethyl)phenyl]- | | | | | | |
| | piperidin-1-yl}methyl)- | | | | | | |
| | 3H-imidazo[4,5- | | | | | | |
| | b]pyridine | | | | | | |
| 8 | 2-{[4-(2- | 0.742 | C19H21FN4 | 324.18 | 325.175 | 1.77 | H |
| | fluorophenyl)piperidin- | | | | | | |
| | 1-yl]methyl}-3-methyl- | | | | | | |
| | 3H-imidazo[4,5- | | | | | | |
| | b]pyridine | | | | | | |
| 9 | (4aS,10bS)-3-[(3- | 2.1 | C21H24N4 | 332.2 | 333.2 | 1.89 | H |
| | methyl-3H-imidazo[4,5- | | | | | | |
| | b]pyridin-2-yl)methyl]- | | | | | | |
| | 1,2,3,4,4a,5,6,10b- | | | | | | |
| | octahydrobenzo[f]isoquinoline | | | | | | |
| 10 | 2-{[4-(2- | 0.963 | C20H24N4O | 336.2 | 337.195 | 1.82 | H |
| | methoxyphenyl)piperidin- | | | | | | |
| | 1-yl]methyl}-3- | | | | | | |
| | methyl-3H-imidazo[4,5- | | | | | | |
| | b]pyridine | | | | | | |
| 11 | 2-{[4-(5-chloro-2-{[(2S)- | 0.548 | C24H31ClN4O | 426.22 | 426.218 | 1.54 | J |
| | 2- | | | | | | |
| | methylbutyl]oxy}phenyl)- | | | | | | |
| | piperidin-1-yl]methyl}- | | | | | | |
| | 1-methyl-1H- | | | | | | |
| | imidazo[4,5-b]pyridine | | | | | | |
| 12 | 2-{[4-(5-chloro-2-{[(2S)- | 1.72 | C24H31ClN4O | 426.22 | 426.218 | 1.57 | J |
| | 2- | | | | | | |
| | methylbutyl]oxy}phenyl)- | | | | | | |
| | piperidin-1-yl]methyl}- | | | | | | |
| | 1-methyl-1H- | | | | | | |
| | imidazo[4,5-c]pyridine | | | | | | |
| 13 | 2-{[4-(5-chloro-2-{[(2S)- | 1.76 | C24H31ClN4O | 426.22 | 426.218 | 1.68 | J |
| | 2- | | | | | | |
| | methylbutyl]oxy}phenyl)- | | | | | | |
| | piperidin-1-yl]methyl}- | | | | | | |
| | 3-methyl-3H- | | | | | | |
| | imidazo[4,5-b]pyridine | | | | | | |
| 14 | 2-{[4-(5-chloro-2- | 0.344 | C23H29ClN4O | 412.2 | 412.2024 | 1.46 | J |
| | isobutoxyphenyl)piperidin- | | | | | | |
| | 1-yl]methyl}-1- | | | | | | |
| | methyl-1H-imidazo[4,5- | | | | | | |
| | b]pyridine | | | | | | |
| 15 | 2-{[4-(5-chloro-2- | 0.804 | C23H29ClN4O | 412.2 | 412.2024 | 1.49 | J |
| | isobutoxyphenyl)piperidin- | | | | | | |
| | 1-yl]methyl}-1- | | | | | | |
| | methyl-1H-imidazo[4,5- | | | | | | |
| | c]pyridine | | | | | | |
| 16 | 2-{[4-(5-chloro-2- | 1.06 | C23H29ClN4O | 412.2 | 412.2024 | 1.6 | J |
| | isobutoxyphenyl)piperidin- | | | | | | |
| | 1-yl]methyl}-3- | | | | | | |
| | methyl-3H-imidazo[4,5- | | | | | | |
| | b]pyridine | | | | | | |
| 17 | 2-{[4-(2-butoxy-5- | 0.786 | C23H29ClN4O | 412.2 | 412.2024 | 1.45 | J |
| | chlorophenyl)piperidin- | | | | | | |
| | 1-yl]methyl}-1-methyl- | | | | | | |
| | 1H-imidazo[4,5- | | | | | | |
| | b]pyridine | | | | | | |
| 18 | 2-{[4-(2-butoxy-5- | 0.407 | C23H29ClN4O | 412.2 | 412.2024 | 1.49 | J |
| | chlorophenyl)piperidin- | | | | | | |
| | 1-yl]methyl}-1-methyl- | | | | | | |
| | 1H-imidazo[4,5- | | | | | | |
| | c]pyridine | | | | | | |
| 19 | 2-{[4-(2-butoxy-5- | 0.647 | C23H29ClN4O | 412.2 | 412.2024 | 1.59 | J |
| | chlorophenyl)piperidin- | | | | | | |
| | 1-yl]methyl}-3-methyl- | | | | | | |
| | 3H-imidazo[4,5- | | | | | | |
| | b]pyridine | | | | | | |
| 20 | 2-({4-[5-chloro-2-(2- | 0.214 | C24H29ClN4O | 424.2 | 424.2024 | 1.45 | J |
| | cyclopropylethoxy)phenyl]- | | | | | | |
| | piperidin-1- | | | | | | |
| | yl}methyl)-1-methyl-1H- | | | | | | |
| | imidazo[4,5-b]pyridine | | | | | | |
| 21 | 2-({4-[5-chloro-2-(2- | 0.399 | C24H29ClN4O | 424.2 | 424.2024 | 1.58 | J |
| | cyclopropylethoxy)phenyl]- | | | | | | |
| | piperidin-1- | | | | | | |
| | yl}methyl)-3-methyl-3H- | | | | | | |
| | imidazo[4,5-b]pyridine | | | | | | |
| 22 | 2-({4-[5-chloro-2-(2- | 0.224 | C24H29ClN4O | 424.2 | 424.2024 | 1.47 | J |
| | cyclopropylethoxy)phenyl]- | | | | | | |
| | piperidin-1- | | | | | | |
| | yl}methyl)-1-methyl-1H- | | | | | | |
| | imidazo[4,5-c]pyridine | | | | | | |
| 23 | 2-({4-[5-chloro-2- | 0.15 | C24H29ClN4O | 424.2 | 424.2024 | 1.49 | J |
| | (cyclobutylmethoxy)phenyl]- | | | | | | |
| | piperidin-1- | | | | | | |
| | yl}methyl-1-methyl-1H- | | | | | | |
| | imidazo[4,5-b]pyridine | | | | | | |
| 24 | 2-({4-[5-chloro-2- | 0.759 | C24H29ClN4O | 424.2 | 424.2024 | 1.52 | J |
| | (cyclobutylmethoxy)phenyl]- | | | | | | |
| | piperidin-1- | | | | | | |
| | yl}methyl)-1-methyl-1H- | | | | | | |
| | imidazo[4,5-c]pyridine | | | | | | |
| 25 | 2-({4-[5-chloro-2- | 0.986 | C24H29ClN4O | 424.2 | 424.2024 | 1.63 | J |
| | (cyclobutylmethoxy)phenyl]- | | | | | | |
| | piperidin-1- | | | | | | |
| | yl}methyl)-3-methyl-3H- | | | | | | |
| | imidazo[4,5-b]pyridine | | | | | | |
| 26 | 2-[(4-{5-chloro-2-[(2- | 0.194 | C24H29ClN4O | 424.2 | 424.2024 | 1.45 | J |
| | methylcyclopropyl)methoxy]- | | | | | | |
| | phenyl}piperidin- | | | | | | |
| | 1-yl)methyl]-1-methyl- | | | | | | |
| | 1H-imidazo[4,5- | | | | | | |
| | b]pyridine | | | | | | |
| 27 | 2-[(4-{5-chloro-2-[(2- | 0.647 | C24H29ClN4O | 424.2 | 424.2024 | 1.47 | J |
| | methylcyclopropyl)methoxy]- | | | | | | |
| | phenyl}piperidin- | | | | | | |
| | 1-yl)methyl]-1-methyl- | | | | | | |
| | 1H-imidazo[4,5- | | | | | | |
| | c]pyridine | | | | | | |
| 28 | 2-[(4-{5-chloro-2-[(2- | 0.96 | C24H29ClN4O | 424.2 | 424.2024 | 1.58 | J |
| | methylcyclopropyl)methoxy]- | | | | | | |
| | phenyl}piperidin- | | | | | | |
| | 1-yl)methyl]-3-methyl- | | | | | | |
| | 3H-imidazo[4,5- | | | | | | |
| | b]pyridine | | | | | | |
| 29 | 2-({4-[5-chloro-2- | 1.53 | C24H29ClN4O2 | 440.2 | 440.1973 | 1.17 | J |
| | (tetrahydrofuran-3- | | | | | | |
| | ylmethoxy)phenyl]piperidin- | | | | | | |
| | 1-yl}methyl)-1- | | | | | | |
| | methyl-1H-imidazo[4,5- | | | | | | |
| | b]pyridine | | | | | | |
| 30 | 2-({4-[5-chloro-2- | 2.5 | C24H29ClN4O2 | 440.2 | 440.1973 | 1.26 | J |
| | (tetrahydrofuran-3- | | | | | | |
| | ylmethoxy)phenyl]piperidin- | | | | | | |
| | 1-yl}methyl)-3- | | | | | | |
| | methyl-3H-imidazo[4,5- | | | | | | |
| | b]pyridine | | | | | | |
| 31 | 2-({4-[5-chloro-2- | 3.24 | C24H29ClN4O2 | 440.2 | 440.1973 | 1.17 | J |
| | (tetrahydrofuran-3- | | | | | | |
| | ylmethoxy)phenyl]piperidin- | | | | | | |
| | 1-yl}methyl)-1- | | | | | | |
| | methyl-1H-imidazo[4,5- | | | | | | |
| | c]pyridine | | | | | | |
| 32 | 2-({4-[5-chloro-2- | 4.58 | C24H29ClN4O2 | 440.2 | 440.1973 | 1.18 | J |
| | (tetrahydrofuran-2- | | | | | | |
| | ylmethoxy)phenyl]piperidin- | | | | | | |
| | 1-yl}methyl)-1- | | | | | | |
| | methyl-1H-imidazo[4,5- | | | | | | |
| | b]pyridine | | | | | | |
| 33 | 2-({4-[5-chloro-2-(prop- | 0.345 | C22H23ClN4O | 394.16 | 394.1556 | 1.02 | J |
| | 2-yn-1- | | | | | | |
| | yloxy)phenyl]piperidin- | | | | | | |
| | 1-yl}methyl)-3-methyl- | | | | | | |
| | 3H-imidazo[4,5- | | | | | | |
| | b]pyridine | | | | | | |
| 34 | 2-({4-[5-chloro-2- | 2.5 | C24H29ClN4O2 | 440.2 | 440.1973 | 1.27 | J |
| | (tetrahydrofuran-2- | | | | | | |
| | ylmethoxy)phenyl]piperidin- | | | | | | |
| | 1-yl}methyl)-3- | | | | | | |
| | methyl-3H-imidazo[4,5- | | | | | | |
| | b]pyridine | | | | | | |
| 35 | 2-({4-[5-chloro-2-(prop- | 0.362 | C22H23ClN4O | 394.16 | 394.1556 | 0.86 | J |
| | 2-yn-1- | | | | | | |
| | yloxy)phenyl]piperidin- | | | | | | |
| | 1-yl}methyl)-1-methyl- | | | | | | |
| | 1H-imidazo[4,5- | | | | | | |
| | c]pyridine | | | | | | |
| 36 | 2-({4-[5-chloro-2- | 0.966 | C25H31ClN4O2 | 454.21 | 454.2129 | 1.29 | J |
| | (tetrahydro-2H-pyran- | | | | | | |
| | 2- | | | | | | |
| | ylmethoxy)phenyl]piperidin- | | | | | | |
| | 1-yl}methyl)-1- | | | | | | |
| | methyl-1H-imidazo[4,5- | | | | | | |
| | b]pyridine | | | | | | |
| 37 | 2-({4-[5-chloro-2- | 1.22 | C25H31ClN4O2 | 454.21 | 454.2129 | 1.41 | J |
| | (tetrahydro-2H-pyran- | | | | | | |
| | 2- | | | | | | |
| | ylmethoxy)phenyl]piperidin- | | | | | | |
| | 1-yl}methyl)-3- | | | | | | |
| | methyl-3H-imidazo[4,5- | | | | | | |
| | b]pyridine | | | | | | |
| 38 | 2-{[4-(5-chloro-2- | 0.392 | C22H27ClN4O | 398.19 | 398.1868 | 1.37 | J |
| | propoxyphenyl)piperidin- | | | | | | |
| | 1-yl]methyl}-1- | | | | | | |
| | methyl-1H-imidazo[4,5- | | | | | | |
| | b]pyridine | | | | | | |
| 39 | 2-{[4-(5-chloro-2- | 0.396 | C22H27ClN4O | 398.19 | 398.1868 | 1.38 | J |
| | propoxyphenyl)piperidin- | | | | | | |
| | 1-yl]methyl}-1- | | | | | | |
| | methyl-1H-imidazo[4,5- | | | | | | |
| | c]pyridine | | | | | | |
| 40 | 2-{[4-(5-chloro-2- | 0.41 | C22H27ClN4O | 398.19 | 398.1868 | 1.49 | J |
| | propoxyphenyl)piperidin- | | | | | | |
| | 1-yl]methyl}-3- | | | | | | |
| | methyl-3H-imidazo[4,5- | | | | | | |
| | b]pyridine | | | | | | |
| 41 | 2-{[4-(5-chloro-2- | 4.81 | C21H25ClN4O | 384.17 | 384.1712 | 1.26 | J |
| | ethoxyphenyl)piperidin- | | | | | | |
| | 1-yl]methyl}-1-methyl- | | | | | | |
| | 1H-imidazo[4,5- | | | | | | |
| | b]pyridine | | | | | | |
| 42 | 2-{[4-(5-chloro-2- | 0.501 | C21H25ClN4O | 384.17 | 384.1712 | 1.38 | J |
| | ethoxyphenyl)piperidin- | | | | | | |
| | 1-yl]methyl}-3-methyl- | | | | | | |
| | 3H-imidazo[4,5- | | | | | | |
| | b]pyridine | | | | | | |
| 43 | 2-{[4-(5-chloro-2- | 0.203 | C20H23ClN4O | 370.16 | 370.1556 | 0.88 | J |
| | methoxyphenyl)piperidin- | | | | | | |
| | 1-yl]methyl}-1- | | | | | | |
| | methyl-1H-imidazo[4,5- | | | | | | |
| | b]pyridine | | | | | | |
| 44 | 2-{[4-(5-chloro-2- | >0.808 | C20H23ClN4O | 370.16 | 370.1556 | 0.98 | J |
| | methoxyphenyl)piperidin- | | | | | | |
| | 1-yl]methyl}-3- | | | | | | |
| | methyl-3H-imidazo[4,5- | | | | | | |
| | b]pyridine | | | | | | |
| 45 | 2-({4-[5-chloro-2-(prop- | 0.0726 | C22H23ClN4O | 394.16 | 394.1556 | 0.91 | J |
| | 2-yn-1- | | | | | | |
| | yloxy)phenyl]piperidin- | | | | | | |
| | 1-yl}methyl)-1-methyl- | | | | | | |
| | 1H-imidazo[4,5- | | | | | | |
| | b]pyridine | | | | | | |
| 46 | 2-{[4-(2- | 1.45 | C20H24N4O | 336.2 | 337.195 | 1.83 | H |
| | methoxyphenyl)piperidin- | | | | | | |
| | 1-yl]methyl}-1- | | | | | | |
| | methyl-1H-imidazo[4,5- | | | | | | |
| | c]pyridine | | | | | | |
| 47 | 1-methyl-2-({4-[4- | 0.168 | C20H21F3N4 | 374.17 | 375.2 | | H, I |
| | (trifluoromethyl)phenyl]- | | | | | | |
| | piperidin-1-yl}methyl)- | | | | | | |
| | 1H-imidazo[4,5- | | | | | | |
| | c]pyridine | | | | | | |
| 48 | 1-methyl-2-({4-[2- | 0.481 | C20H21F3N4 | 374.17 | 375.172 | 2.24 | H |
| | (trifluoromethyl)phenyl]- | | | | | | |
| | piperidin-1-yl}methyl)- | | | | | | |
| | 1H-imidazo[4,5- | | | | | | |
| | c]pyridine | | | | | | |
| 49 | 2-{[4-(2- | 1.05 | C19H21FN4 | 324.18 | 325.175 | 1.69 | H |
| | fluorophenyl)piperidin- | | | | | | |
| | 1-yl]methyl}-1-methyl- | | | | | | |
| | 1H-imidazo[4,5- | | | | | | |
| | c]pyridine | | | | | | |
| 50 | 3-methyl-2-({4-[2- | 0.406 | C20H21F3N4 | 374.17 | 375.172 | 2.95 | H |
| | (trifluoromethyl)phenyl]- | | | | | | |
| | piperidin-1-yl}methyl)- | | | | | | |
| | 3H-imidazo[4,5- | | | | | | |
| | b]pyridine | | | | | | |
| 51 | 2-{[4-(2- | 1.54 | C20H24N4O | 336.2 | 337.195 | 2.06 | H |
| | methoxyphenyl)piperidin- | | | | | | |
| | 1-yl]methyl}-1- | | | | | | |
| | methyl-1H-imidazo[4,5- | | | | | | |
| | b]pyridine | | | | | | |
| 52 | 1-methyl-2-({4-[4- | 0.0479 | C20H21F3N4 | 374.17 | 375.1 | | H, I |
| | (trifluoromethyl)phenyl]- | | | | | | |
| | piperidin-1-yl}methyl)- | | | | | | |
| | 1H-imidazo[4,5- | | | | | | |
| | b]pyridine | | | | | | |
| 53 | 1-methyl-2-({4-[2- | 0.105 | C20H21F3N4 | 374.17 | 374.1714 | 1.26 | H |
| | (trifluoromethyl)phenyl]- | | | | | | |
| | piperidin-1-yl}methyl)- | | | | | | |
| | 1H-imidazo[4,5- | | | | | | |
| | b]pyridine | | | | | | |
| 54 | 2-{[4-(2- | 1.59 | C19H21FN4 | 324.17 | 325.1 | | H, I |
| | fluorophenyl)piperidin- | | | | | | |
| | 1-yl]methyl}-1-methyl- | | | | | | |
| | 1H-imidazo[4,5- | | | | | | |
| | b]pyridine | | | | | | |
| 55 | 2-({4-[2-methoxy-5- | 0.211 | C21H23F3N4O | 404.18 | 422.43 | 3.36 | H |
| | (trifluoromethyl)phenyl]- | | | | | | |
| | piperidin-1-yl}methyl)- | | | | | | |
| | 1-methyl-1H- | | | | | | |
| | imidazo[4,5-b]pyridine | | | | | | |
| 56 | 2-{[(3R,4R)-4-(2- | 3.82 | C22H28N4O | 364.23 | 365.53 | 2.29 | H |
| | methoxyphenyl)-3,4- | | | | | | |
| | dimethylpiperidin-1- | | | | | | |
| | yl]methyl}-1-methyl-1H- | | | | | | |
| | imidazo[4,5-b]pyridine | | | | | | |
| 57 | 2-({4-[2-fluoro-5- | 0.448 | C20H20F4N4 | 392.16 | 393.2 | 3.29 | H |
| | (trifluoromethyl)phenyl]- | | | | | | |
| | piperidin-1-yl}methyl)- | | | | | | |
| | 1-methyl-1H- | | | | | | |
| | imidazo[4,5-c]pyridine | | | | | | |
| 58 | 2-({4-[2-fluoro-5- | 5.95 | C20H20F4N4 | 392.16 | 393.26 | 3.43 | H |
| | (trifluoromethyl)phenyl]- | | | | | | |
| | piperidin-1-yl}methyl)- | | | | | | |
| | 1-methyl-1H- | | | | | | |
| | imidazo[4,5-b]pyridine | | | | | | |
| 59 | 2-{[4-(2- | 0.349 | C21H26N4O | 350.21 | 351.25 | 2.71 | I |
| | methoxyphenyl)-3- | | | | | | |
| | methylpiperidin-1- | | | | | | |
| | yl]methyl}-3-methyl-3H- | | | | | | |
| | imidazo[4,5-b]pyridine | | | | | | |
| 60 | 2-{[4-(2- | 0.302 | C21H26N4O | 350.21 | 351.25 | 1.99 | I |
| | methoxyphenyl)-3- | | | | | | |
| | methylpiperidin-1- | | | | | | |
| | yl]methyl}-1-methyl-1H- | | | | | | |
| | imidazo[4,5-b]pyridine | | | | | | |
| 61 | 2-{[4-(2,5- | 0.29 | C21H26N4 | 334.22 | 335.1 | | I |
| | dimethylphenyl)piperidin- | | | | | | |
| | 1-yl]methyl}-1- | | | | | | |
| | methyl-1H-imidazo[4,5- | | | | | | |
| | b]pyridine | | | | | | |
| 62 | 1-methyl-2-{[4-(3- | 0.617 | C20H24N4 | 320.2 | 321.1 | | I |
| | methylphenyl)piperidin- | | | | | | |
| | 1-yl]methyl}-1H- | | | | | | |
| | imidazo[4,5-b]pyridine | | | | | | |
| 63 | 2-({4-[2-methoxy-4- | 0.834 | C21H23F3N4O | 404.18 | 405.2 | 2.69 | H, I |
| | (trifluoromethyl)phenyl]- | | | | | | |
| | piperidin-1-yl}methyl)- | | | | | | |
| | 1-methyl-1H- | | | | | | |
| | imidazo[4,5-c]pyridine | | | | | | |
| 64 | 2-({4-[2-methoxy-4- | 0.0111 | C21H23F3N4O | 404.18 | 405.2 | | H, I |
| | (trifluoromethyl)phenyl]- | | | | | | |
| | piperidin-1-yl}methyl)- | | | | | | |
| | 1-methyl-1H- | | | | | | |
| | imidazo[4,5-b]pyridine | | | | | | |
| 65 | 2-({4-[3,5- | 0.144 | C21H20F6N4 | 442.16 | 443.1 | | I |
| | bis(trifluoromethyl)phenyl]- | | | | | | |
| | piperidin-1- | | | | | | |
| | yl}methyl)-1-methyl-1H- | | | | | | |
| | imidazo[4,5-b]pyridine | | | | | | |
| 66 | 2-{[4-(4- | 1.26 | C19H21FN4 | 324.17 | 325.29 | 2.69 | H, I |
| | fluorophenyl)piperidin- | | | | | | |
| | 1-yl]methyl}-3-methyl- | | | | | | |
| | 3H-imidazo[4,5- | | | | | | |
| | b]pyridine | | | | | | |
| 67 | 2-{[4-(4- | 2.36 | C19H21FN4 | 324.17 | 325 | | H, I |
| | fluorophenyl)piperidin- | | | | | | |
| | 1-yl]methyl}-1-methyl- | | | | | | |
| | 1H-imidazo[4,5- | | | | | | |
| | b]pyridine | | | | | | |
| 68 | 2-{[4-(4- | 1.8 | C19H21FN4 | 324.17 | 325.27 | 2.29 | H, I |
| | fluorophenyl)piperidin- | | | | | | |
| | 1-yl]methyl}-1-methyl- | | | | | | |
| | 1H-imidazo[4,5- | | | | | | |
| | c]pyridine | | | | | | |
| 69 | 2-{[4-(4-fluoro-2- | 0.353 | C20H23FN4 | 338.19 | 339.1 | | I |
| | methylphenyl)piperidin- | | | | | | |
| | 1-yl]methyl}-1-methyl- | | | | | | |
| | 1H-imidazo[4,5- | | | | | | |
| | b]pyridine | | | | | | |
| 70 | 1-methyl-2-({3-methyl- | 0.111 | C21H23F3N4 | 388.19 | 389.2 | | I |
| | 4-[4- | | | | | | |
| | (trifluoromethyl)phenyl]- | | | | | | |
| | piperidin-1-yl}methyl)- | | | | | | |
| | 1H-imidazo[4,5- | | | | | | |
| | b]pyridine | | | | | | |
| 71 | 1-methyl-2-({cis-3- | 0.075 | C21H23F3N4 | 388.19 | 389.2 | | I |
| | methyl-4-[4- | | | | | | |
| | (trifluoromethyl)phenyl]- | | | | | | |
| | piperidin-1-yl}methyl)- | | | | | | |
| | 1H-imidazo[4,5- | | | | | | |
| | c]pyridine | | | | | | |
| 72 | 2-{[4-(2-fluorophenyl)- | 0.301 | C20H23FN4 | 338.19 | 339.4 | | I |
| | 3-methylpiperidin-1- | | | | | | |
| | yl]methyl}-1-methyl-1H- | | | | | | |
| | imidazo[4,5-b]pyridine | | | | | | |
| 73 | 2-{[Cis-4-(2- | 0.242 | C20H23FN4 | 338.19 | 339.3 | | I |
| | fluorophenyl)-3- | | | | | | |
| | methylpiperidin-1- | | | | | | |
| | yl]methyl}-1-methyl-1H- | | | | | | |
| | imidazo[4,5-c]pyridine | | | | | | |
| 74 | 2-{[4-(2,5- | 0.782 | C19H20F2N4 | 342.17 | 342.1 | | I |
| | difluorophenyl)piperidin- | | | | | | |
| | 1-yl]methyl}-1-methyl- | | | | | | |
| | 1H-imidazo[4,5- | | | | | | |
| | b]pyridine | | | | | | |
| 75 | 2-{[4-(5-chloro-2- | 0.324 | C19H20ClFN4 | 358.14 | 359 | | I |
| | fluorophenyl)piperidin- | | | | | | |
| | 1-yl]methyl}-1-methyl- | | | | | | |
| | 1H-imidazo[4,5- | | | | | | |
| | b]pyridine | | | | | | |
| 76 | 2-({3,3-dimethyl-4-[4- | 0.745 | C22H25F3N4 | 402.2 | 403.4 | | I |
| | (trifluoromethyl)phenyl]- | | | | | | |
| | piperidin-1-yl}methyl)- | | | | | | |
| | 1-methyl-1H- | | | | | | |
| | imidazo[4,5-b]pyridine | | | | | | |
| 77 | 2-{1-[(1-methyl-1H- | >5.70 | C20H21N5 | 331.18 | 332.3 | | I |
| | imidazo[4,5-b]pyridin-2- | | | | | | |
| | yl)methyl]piperidin-4- | | | | | | |
| | yl}benzonitrile | | | | | | |
| 78 | 2-{[4-(3- | 0.88 | C19H21ClN4 | 340.15 | 341.2 | | I |
| | chlorophenyl)piperidin- | | | | | | |
| | 1-yl]methyl}-1-methyl- | | | | | | |
| | 1H-imidazo[4,5- | | | | | | |
| | b]pyridine | | | | | | |
| 79 | 2-{[4-(3-tert- | 0.124 | C23H30N4 | 362.25 | 363.3 | | I |
| | butylphenyl)piperidin-1- | | | | | | |
| | yl]methyl}-1-methyl-1H- | | | | | | |
| | imidazo[4,5-b]pyridine | | | | | | |
| 80 | 2-{[4-(4- | >9.11 | C19H21ClN4 | 340.15 | 342.2 | | I |
| | chlorophenyl)piperidin- | | | | | | |
| | 1-yl]methyl}-1-methyl- | | | | | | |
| | 1H-imidazo[4,5- | | | | | | |
| | b]pyridine | | | | | | |
| 81 | 2-{[4-(4- | 9.77 | C20H24N4O | 336.2 | 337.3 | | I |
| | methoxyphenyl)piperidin- | | | | | | |
| | 1-yl]methyl}-1- | | | | | | |
| | methyl-1H-imidazo[4,5- | | | | | | |
| | b]pyridine | | | | | | |
| 82 | 2-{[4-(2,4- | 0.251 | C19H20F2N4 | 342.17 | 343.2 | | I |
| | difluorophenyl)piperidin- | | | | | | |
| | 1-yl]methyl}-1-methyl- | | | | | | |
| | 1H-imidazo[4,5- | | | | | | |
| | b]pyridine | | | | | | |
| 83 | 1-methyl-2-({4-[2- | 0.221 | C20H21F3N4O | 390.17 | 391.2 | | I |
| | (trifluoromethoxy)phenyl]- | | | | | | |
| | piperidin-1- | | | | | | |
| | yl}methyl)-1H- | | | | | | |
| | imidazo[4,5-b]pyridine | | | | | | |
| 84 | 2-{[4-(2,4- | 0.218 | C19H20F2N4 | 342.7 | 343.3 | | I |
| | difluorophenyl)piperidin- | | | | | | |
| | 1-yl]methyl}-1-methyl- | | | | | | |
| | 1H-imidazo[4,5- | | | | | | |
| | c]pyridine | | | | | | |
| 85 | 2-{[4-(2,4- | 0.199 | C19H20F2N4 | 342.17 | 343.2 | | I |
| | difluorophenyl)piperidin- | | | | | | |
| | 1-yl]methyl}-3-methyl- | | | | | | |
| | 3H-imidazo[4,5- | | | | | | |
| | b]pyridine | | | | | | |
| 86 | 1-[(1-methyl-1H- | >2.75 | C20H21F3N4O | 390.17 | 391.1 | | I |
| | imidazo[4,5-b]pyridin-2- | | | | | | |
| | yl)methyl]-4-[4- | | | | | | |
| | (trifluoromethyl)phenyl]- | | | | | | |
| | piperidin-3-ol | | | | | | |
| 87 | 2-{[4-(4-fluoro-2- | 0.212 | C20H23FN4O | 354.19 | 355.2 | | I |
| | methoxyphenyl)piperidin- | | | | | | |
| | 1-yl]methyl}-1- | | | | | | |
| | methyl-1H-imidazo[4,5- | | | | | | |
| | b]pyridine | | | | | | |
| 88 | 2-{[4-(4-fluoro-2- | 0.372 | C20H23FN4O | 354.19 | 355.4 | | I |
| | methoxyphenyl)piperidin- | | | | | | |
| | 1-yl]methyl}-1- | | | | | | |
| | methyl-1H-imidazo[4,5- | | | | | | |
| | c]pyridine | | | | | | |
| 89 | 1-methyl-2-{[4-(2- | 0.823 | C20H24N4 | 320.2 | 321.2 | | I |
| | methylphenyl)piperidin- | | | | | | |
| | 1-yl]methyl}-1H- | | | | | | |
| | imidazo[4,5-b]pyridine | | | | | | |
| 90 | 2-{[4-(3- | 1.99 | C19H21FN4 | 324.17 | 325.1 | | I |
| | fluorophenyl)piperidin- | | | | | | |
| | 1-yl]methyl}-1-methyl- | | | | | | |
| | 1H-imidazo[4,5- | | | | | | |
| | b]pyridine | | | | | | |
| 91 | 2-{[4-(3,5- | 0.985 | C19H20F2N4 | 342.17 | 343.2 | | I |
| | difluorophenyl)piperidin- | | | | | | |
| | 1-yl]methyl}-1-methyl- | | | | | | |
| | 1H-imidazo[4,5- | | | | | | |
| | b]pyridine | | | | | | |
| 92 | 2-{[4-(3-fluoro-4- | 2.8 | C20H23FN4O | 354.19 | 355.2 | | I |
| | methoxyphenyl)piperidin- | | | | | | |
| | 1-yl]methyl}-1- | | | | | | |
| | methyl-1H-imidazo[4,5- | | | | | | |
| | b]pyridine | | | | | | |
| 93 | 3-methyl-2-({4-[4- | 0.196 | C20H21F3N4 | 374.17 | 375.2 | | I |
| | (trifluoromethyl)phenyl]- | | | | | | |
| | piperidin-1-yl}methyl)- | | | | | | |
| | 3H-imidazo[4,5- | | | | | | |
| | c]pyridine | | | | | | |
| 94 | 2-{[4-(2- | 1.35 | C19H21FN4 | 324.17 | 325.4 | | I |
| | fluorophenyl)piperidin- | | | | | | |
| | 1-yl]methyl}-3-methyl- | | | | | | |
| | 3H-imidazo[4,5- | | | | | | |
| | c]pyridine | | | | | | |
| 95 | 2-{[4-(2- | 1.38 | C20H24N4O | 336.2 | 337.4 | | I |
| | methoxyphenyl)piperidin- | | | | | | |
| | 1-yl]methyl}-3- | | | | | | |
| | methyl-3H-imidazo[4,5- | | | | | | |
| | c]pyridine | | | | | | |
| 96 | 2-{[4-(4- | 5.2 | C19H21FN4 | 324.17 | 325.4 | | I |
| | fluorophenyl)piperidin- | | | | | | |
| | 1-yl]methyl}-3-methyl- | | | | | | |
| | 3H-imidazo[4,5- | | | | | | |
| | c]pyridine | | | | | | |
| 97 | 2-{[4-(2-fluoro-6- | 0.756 | C20H23FN4O | 354.19 | 355.1 | | I |
| | methoxypheny)piperidin- | | | | | | |
| | 1-yl]methyl}-1- | | | | | | |
| | methyl-1H-imidazo[4,5- | | | | | | |
| | b]pyridine | | | | | | |
| 98 | 2-{[4-(4- | 2.27 | C21H26N4O | 350.21 | 351.3 | | I |
| | ethoxyphenyl)piperidin- | | | | | | |
| | 1-yl]methyl}-1-methyl- | | | | | | |
| | 1H-imidazo[4,5- | | | | | | |
| | b]pyridine | | | | | | |
| 99 | 2-({4-fluoro-4-[4- | 0.434 | C20H20F4N4 | 392.16 | 393.1 | | I |
| | (trifluoromethyl)phenyl]- | | | | | | |
| | piperidin-1-yl}methyl)- | | | | | | |
| | 1-methyl-1H- | | | | | | |
| | imidazo[4,5-b]pyridine | | | | | | |
| 100 | 2-({3-fluoro-4-[4- | 0.315 | C20H20F4N4 | 392.16 | 393.2 | | I |
| | (trifluoromethyl)phenyl]- | | | | | | |
| | piperidin-1-yl}methyl)- | | | | | | |
| | 1-methyl-1H- | | | | | | |
| | imidazo[4,5-b]pyridine | | | | | | |
| 101 | 2-{[4-(3-chloro-4- | 0.313 | C19H20ClFN4 | 358.14 | 359.3 | | I |
| | fluorophenyl)piperidin- | | | | | | |
| | 1-yl]methyl}-1-methyl- | | | | | | |
| | 1H-imidazo[4,5- | | | | | | |
| | b]pyridine | | | | | | |
| 102 | 2-{[4-(3-chloro-4- | 0.482 | C19H20ClFN4 | 358.14 | 359.3 | | I |
| | fluorophenyl)piperidin- | | | | | | |
| | 1-yl]methyl}-1-methyl- | | | | | | |
| | 1H-imidazo[4,5- | | | | | | |
| | c]pyridine | | | | | | |
| 103 | 2-{[4-(3-chloro-4- | 0.367 | C19H20ClFN4 | 358.14 | 359.3 | | I |
| | fluorophenyl)piperidin- | | | | | | |
| | 1-yl]methyl}-3-methyl- | | | | | | |
| | 3H-imidazo[4,5- | | | | | | |
| | b]pyridine | | | | | | |
| 104 | 1-methyl-2-{[4-(4- | 0.71 | C20H24N4 | 320.2 | 321 | | I |
| | methylphenyl)piperidin- | | | | | | |
| | 1-yl]methyl}-1H- | | | | | | |
| | imidazo[4,5-b]pyridine | | | | | | |
| 105 | 3-{1-[(1-methyl-1H- | 3.66 | C24H27N7O | 429.23 | 430.1 | | I |
| | imidazo[4,5-b]pyridin-2- | | | | | | |
| | yl)methyl]piperidin-4- | | | | | | |
| | yl}-N-(5-methyl-1H- | | | | | | |
| | pyrazol-3-yl)benzamide | | | | | | |
| 106 | 2-{[4-(4-chloro-3- | 0.19 | C19H20ClFN4 | 358.14 | 359.2 | | I |
| | fluorophenyl)piperidin- | | | | | | |
| | 1-yl]methyl}-1-methyl- | | | | | | |
| | 1H-imidazo[4,5- | | | | | | |
| | b]pyridine | | | | | | |
| 107 | 2-{[4-(4-chloro-3- | 0.315 | C19H20ClFN4 | 358.14 | 359.2 | | I |
| | fluorophenyl)piperidin- | | | | | | |
| | 1-yl]methyl}-1-methyl- | | | | | | |
| | 1H-imidazo[4,5- | | | | | | |
| | c]pyridine | | | | | | |
| 108 | 2-{[4-(2,3- | 0.307 | C19H20F2N4 | 342.17 | 343.2 | 1.42 | I |
| | difluorophenyl)piperidin- | | | | | | |
| | 1-yl]methyl}-1-methyl- | | | | | | |
| | 1H-imidazo[4,5- | | | | | | |
| | b]pyridine | | | | | | |
| 109 | 2-{[4-(4-chloro-3- | 0.378 | C19H20ClFN4 | 358.14 | 359.2 | 1.7 | I |
| | fluorophenyl)piperidin- | | | | | | |
| | 1-yl]methyl}-3-methyl- | | | | | | |
| | 3H-imidazo[4,5- | | | | | | |
| | b]pyridine | | | | | | |
| 110 | 2-{[4-(4-chloro-2- | 0.0271 | C19H20ClFN4 | 358.14 | 359.1 | 1.46 | I |
| | fluorophenyl)piperidin- | | | | | | |
| | 1-yl]methyl}-1-methyl- | | | | | | |
| | 1H-imidazo[4,5- | | | | | | |
| | b]pyridine | | | | | | |
| 111 | 2-{[Cis-4-(3-chloro-4- | 0.203 | C20H22ClFN4 | 372.15 | 373.1 | 1.7 | I |
| | fluorophenyl)-3- | | | | | | |
| | methylpiperidin-1- | | | | | | |
| | yl]methyl}-1-methyl-1H- | | | | | | |
| | imidazo[4,5-b]pyridine | | | | | | |
| 112 | 2-{[4-(4-chloro-2- | 0.0585 | C19H20ClFN4 | 358.14 | 359.3 | | I |
| | fluorophenyl)piperidin- | | | | | | |
| | 1-yl]methyl}-3-methyl- | | | | | | |
| | 3H-imidazo[4,5- | | | | | | |
| | b]pyridine | | | | | | |
| 113 | 2-{[4-(4-chloro-2- | 0.081 | C19H20ClFN4 | 358.14 | 359.1 | 1.3 | I |
| | fluorophenyl)piperidin- | | | | | | |
| | 1-yl]methyl}-1-methyl- | | | | | | |
| | 1H-imidazo[4,5- | | | | | | |
| | c]pyridine | | | | | | |
| 114 | 2-({Cis-4-[2-methoxy-4- | 0.0428 | C22H25F3N4O | 418.2 | 419.1 | | I |
| | (trifluoromethyl)phenyl]- | | | | | | |
| | 3-methylpiperidin-1- | | | | | | |
| | yl}methyl)-1-methyl-1H- | | | | | | |
| | imidazo[4,5-b]pyridine | | | | | | |
| 115 | 2-{[Cis-4-(4-chloro-2- | 0.0195 | C20H22ClFN4 | 372.15 | 373.1 | 1.72 | I |
| | fluorophenyl)-3- | | | | | | |
| | methylpiperidin-1- | | | | | | |
| | yl]methyl}-1-methyl-1H- | | | | | | |
| | imidazo[4,5-b]pyridine | | | | | | |
| 116 | 2-{[Cis-4-(4-chloro-2- | 0.0498 | C20H22ClFN4 | 372.15 | 373.3 | 1.56 | I |
| | fluorophenyl)-3- | | | | | | |
| | methylpiperidin-1- | | | | | | |
| | yl]methyl}-1-methyl-1H- | | | | | | |
| | imidazo[4,5-c]pyridine | | | | | | |
| 117 | 2-{[Cis-4-(2- | >0.359 | C20H23FN4 | 338.19 | 339.3 | 0.91 | I |
| | fluorophenyl)-3- | | | | | | |
| | methylpiperidin-1- | | | | | | |
| | yl]methyl}-1-methyl-1H- | | | | | | |
| | imidazo[4,5-c]pyridine | | | | | | |
| 118 | 1-methyl-2-({Cis-3- | 0.0334 | C21H23F3N4 | 388.19 | 389.3 | | K |
| | methyl-4-[4- | | | | | | |
| | (trifluoromethyl)phenyl]- | | | | | | |
| | piperidin-1-yl}methyl)- | | | | | | |
| | 1H-imidazo[4,5- | | | | | | |
| | b]pyridine, enantiomer | | | | | | |
| | #1 | | | | | | |
| 119 | 1-methyl-2-({Cis-3- | >1.99 | C21H23F3N4 | 388.19 | 389.3 | | K |
| | methyl-4-[4- | | | | | | |
| | (trifluoromethyl)phenyl]- | | | | | | |
| | piperidin-1-yl}methyl)- | | | | | | |
| | 1H-imidazo[4,5- | | | | | | |
| | b]pyridine, enantiomer | | | | | | |
| | #2 | | | | | | |
| 120 | 2-{[Cis-4-(4- | 0.639 | C22H28N4O | 364.23 | 365.1 | 1.51 | I |
| | ethoxyphenyl)-3- | | | | | | |
| | methylpiperidin-1- | | | | | | |
| | yl]methyl}-1-methyl-1H- | | | | | | |
| | imidazo[4,5-b]pyridine | | | | | | |
| 121 | 2-{[Cis-4-(3,4- | 0.753 | C20H22F2N4 | 356.18 | 357.1 | 1.29 | I |
| | difluorophenyl)-3- | | | | | | |
| | methylpiperidin-1- | | | | | | |
| | yl]methyl}-1-methyl-1H- | | | | | | |
| | imidazo[4,5-b]pyridine | | | | | | |
| 122 | 2-{[Cis-4-(4-chloro-2- | 0.0288 | C20H22ClFN4 | 372.15 | 373.1 | 1.9 | I |
| | fluorophenyl)-3- | | | | | | |
| | methylpiperidin-1- | | | | | | |
| | yl]methyl}-1-methyl-1H- | | | | | | |
| | imidazo[4,5-b]pyridine, | | | | | | |
| | enantiomer #1 | | | | | | |
| 123 | 2-{[(3R,4R)-4-(4- | >1.81 | C20H22ClFN4 | 372.15 | 373.1 | 1.9 | I |
| | chloro-2-fluorophenyl)- | | | | | | |
| | 3-methylpiperidin-1- | | | | | | |
| | yl]methyl}-1-methyl-1H- | | | | | | |
| | imidazo[4,5-b]pyridine | | | | | | |
| 124 | 2-{[Cis-4-(2,4- | 0.118 | C20H22F2N4 | 356.18 | 357.1 | | I |
| | difluorophenyl)-3- | | | | | | |
| | methylpiperidin-1- | | | | | | |
| | yl]methyl}-1-methyl-1H- | | | | | | |
| | imidazo[4,5-b]pyridine | | | | | | |
| 125 | 2-{[Cis-4-(2,4- | 10.3 | C20H22F2N4 | 356.18 | 357.1 | | I |
| | difluorophenyl)-3- | | | | | | |
| | methylpiperidin-1- | | | | | | |
| | yl]methyl}-1-methyl-1H- | | | | | | |
| | imidazo[4,5-c]pyridine | | | | | | |
| 126 | 2-({4-[4- | 0.0626 | C22H23F3N4 | 400.19 | 401.1 | 2.16 | I |
| | (trifluoromethyl)phenyl]- | | | | | | |
| | piperidin-1-yl}methyl)- | | | | | | |
| | 5,6-dihydro-4H- | | | | | | |
| | imidazo[4,5,1-ij]-1,7- | | | | | | |
| | naphthyridine | | | | | | |
| 127 | 2-{[Cis-4-(2,4- | 0.0471 | C22H24F2N4 | 382.2 | 383.1 | | I |
| | difluorophenyl)-3- | | | | | | |
| | methylpiperidin-1- | | | | | | |
| | yl]methyl}-5,6-dihydro- | | | | | | |
| | 4H-imidazo[4,5,1-ij]- | | | | | | |
| | 1,7-naphthyridine | | | | | | |
| 128 | 2-{[Cis-4-(3-chloro-4- | 0.134 | C22H24ClFN4 | 398.17 | 399 | 2.16 | I |
| | fluorophenyl)-3- | | | | | | |
| | methylpiperidin-1- | | | | | | |
| | yl]methyl}-5,6-dihydro- | | | | | | |
| | 4H-imidazo[4,5,1-ij]- | | | | | | |
| | 1,7-naphthyridine | | | | | | |
| 129 | 2-{[Cis-4-(4-chloro-2- | 0.0125 | C22H24ClFN4 | 398.17 | 399 | 1.9 | I |
| | fluorophenyl)-3- | | | | | | |
| | methylpiperidin-1- | | | | | | |
| | yl]methyl}-5,6-dihydro- | | | | | | |
| | 4H-imidazo[4,5,1-ij]- | | | | | | |
| | 1,7-naphthyridine | | | | | | |
| 130 | 2-({Cis-4-[2-methoxy-4- | 0.787 | C22H25F3N4O | 418.2 | 419.1 | | I |
| | (trifluoromethyl)phenyl]- | | | | | | |
| | 3-methylpiperidin-1- | | | | | | |
| | yl}methyl)-1-methyl-1H- | | | | | | |
| | imidazo[4,5-b]pyridine, | | | | | | |
| | Enantiomer #1 | | | | | | |
| 131 | 2-({Cis-4-[2-methoxy-4- | 0.0219 | C22H25F3N4O | 418.2 | 419.1 | | I |
| | (trifluoromethyl)phenyl]- | | | | | | |
| | 3-methylpiperidin-1- | | | | | | |
| | yl}methyl)-1-methyl-1H- | | | | | | |
| | imidazo[4,5-b]pyridine, | | | | | | |
| | Enantiomer #2 | | | | | | |
| 132 | 2-{[Cis-4-(4-fluoro-2- | 0.0785 | C21H25FN4O | 368.2 | 369.2 | | I |
| | methoxyphenyl)-3- | | | | | | |
| | methylpiperidin-1- | | | | | | |
| | yl]methyl}-1-methyl-1H- | | | | | | |
| | imidazo[4,5-b]pyridine | | | | | | |
| 133 | 2-{[Cis-4-(4-fluoro-2- | 0.0537 | C21H25FN4O | 368.2 | 369.1 | 1.6 | I |
| | methoxyphenyl)-3- | | | | | | |
| | methylpiperidin-1- | | | | | | |
| | yl]methyl}-1-methyl-1H- | | | | | | |
| | imidazo[4,5-c]pyridine | | | | | | |
| 134 | 2-{[Cis-4-(4-fluoro-2- | 0.0302 | C23H27FN4O | 394.22 | 395.1 | 1.9 | I |
| | methoxyphenyl)-3- | | | | | | |
| | methylpiperidin-1- | | | | | | |
| | yl]methyl}-5,6-dihydro- | | | | | | |
| | 4H-imidazo[4,5,1-ij]- | | | | | | |
| | 1,7-naphthyridine | | | | | | |
| 135 | 2-({4-[2-methoxy-4- | <0.00543 | C23H25F3N4O | 430.2 | 431.1 | 2.42 | I |
| | (trifluoromethyl)phenyl]- | | | | | | |
| | piperidin-1-yl}methyl)- | | | | | | |
| | 5,6-dihydro-4H- | | | | | | |
| | imidazo[4,5,1-ij]-1,7- | | | | | | |
| | naphthyridine | | | | | | |
| 138 | 2-({Cis-4-[2-fluoro-4- | <0.00925 | C21H22F4N4 | 406.18 | 407.1 | 2.1 | K |
| | (trifluoromethyl)phenyl]- | | | | | | |
| | 3-methylpiperidin-1- | | | | | | |
| | yl}methyl)-1-methyl-1H- | | | | | | |
| | imidazo[4,5-b]pyridine, | | | | | | |
| | enantiomer #1 | | | | | | |
| 139 | 2-({Cis-4-[2-fluoro-4- | 0.993 | C21H22F4N4 | 406.18 | 407.1 | 2.1 | K |
| | (trifluoromethyl)phenyl]- | | | | | | |
| | 3-methylpiperidin-1- | | | | | | |
| | yl}methyl)-1-methyl-1H- | | | | | | |
| | imidazo[4,5-b]pyridine, | | | | | | |
| | enantiomer #2 | | | | | | |
| 140 | 2-{[Cis-4-(4-chloro-2- | 2.05 | C20H22ClFN4 | 372.15 | 373.1 | 1.9 | K |
| | fluorophenyl)-3- | | | | | | |
| | methylpiperidin-1- | | | | | | |
| | yl]methyl}-1-methyl-1H- | | | | | | |
| | imidazo[4,5-c]pyridine, | | | | | | |
| | enantiomer #1 | | | | | | |
| 141 | 2-({cis-4-[2-methoxy-4- | 1.31 | C22H25F3N4O | 418.2 | 419.2 | 1.8 | K |
| | (trifluoromethyl)phenyl]- | | | | | | |
| | 3-methylpiperidin-1- | | | | | | |
| | yl}methyl)-1-methyl-1H- | | | | | | |
| | imidazo[4,5-c]pyridine, | | | | | | |
| | enantiomer #1 | | | | | | |
| 142 | 2-({Cis-4-[2-methoxy-4- | >0.314 | C22H25F3N4O | 418.2 | 419.2 | 1.8 | K |
| | (trifluoromethyl)phenyl]- | | | | | | |
| | 3-methylpiperidin-1- | | | | | | |
| | yl}methyl)-1-methyl-1H- | | | | | | |
| | imidazo[4,5-c]pyridine, | | | | | | |
| | enantiomer #2 | | | | | | |
| 143 | 2-({Cis-4-[2-fluoro-4- | 1.18 | C21H22F4N4 | 406.18 | 407.1 | 1.9 | K |
| | (trifluoromethyl)phenyl]- | | | | | | |
| | 3-methylpiperidin-1- | | | | | | |
| | yl}methyl)-1-methyl-1H- | | | | | | |
| | imidazo[4,5-c]pyridine, | | | | | | |
| | enantiomer #1 | | | | | | |
| 144 | 2-({Cis-4-[2-fluoro-4- | 0.023 | C21H22F4N4 | 406.42 | 407.1 | 1.9 | K |
| | (trifluoromethyl)phenyl]- | | | | | | |
| | 3-methylpiperidin-1- | | | | | | |
| | yl}methyl)-1-methyl-1H- | | | | | | |
| | imidazo[4,5-c]pyridine, | | | | | | |
| | Enantiomer #2 | | | | | | |
| 145 | 2-({Cis-4-[2-fluoro-4- | 0.026 | C21H22F4N4O | 422.17 | 423.3 | 2 | I |
| | (trifluoromethoxy)phenyl]- | | | | | | |
| | 3-methylpiperidin-1- | | | | | | |
| | yl}methyl)-1-methyl-1H- | | | | | | |
| | imidazo[4,5-b]pyridine | | | | | | |
| 146 | 2-({Cis-4-[2-fluoro-4- | 0.0555 | C21H22F4N4O | 422.17 | 423.3 | 1.6 | I |
| | (trifluoromethoxy)phenyl]- | | | | | | |
| | 3-methylpiperidin-1- | | | | | | |
| | yl}methyl)-1-methyl-1H- | | | | | | |
| | imidazo[4,5-c]pyridine | | | | | | |
| 147 | 2-({Cis-4-[3-fluoro-4- | 0.0413 | C21H22F4N4 | 406.18 | 407.3 | 1.9 | I |
| | (trifluoromethyl)phenyl]- | | | | | | |
| | 3-methylpiperidin-1- | | | | | | |
| | yl}methyl)-1-methyl-1H- | | | | | | |
| | imidazo[4,5-b]pyridine | | | | | | |
| 148 | 2-({Cis-4-[3-fluoro-4- | 0.0714 | C21H22F4N4 | 406.18 | 407.3 | 1.6 | I |
| | (trifluoromethyl)phenyl]- | | | | | | |
| | 3-methylpiperidin-1- | | | | | | |
| | yl}methyl)-1-methyl-1H- | | | | | | |
| | imidazo[4,5-c]pyridine | | | | | | |
| 149 | 2-({Cis-4-[2,4- | 0.0248 | C22H22F6N4 | 456.17 | 457.3 | 2.2 | I |
| | bis(trifluoromethyl)phenyl]- | | | | | | |
| | 3-methylpiperidin-1- | | | | | | |
| | yl}methyl)-1-methyl-1H- | | | | | | |
| | imidazo[4,5-b]pyridine | | | | | | |
| 150 | 2-({Cis-4-[2,4- | 0.207 | C22H22F6N4 | 456.17 | 457.3 | 2 | I |
| | bis(trifluoromethyl)phenyl]- | | | | | | |
| | 3-methylpiperidin-1- | | | | | | |
| | yl}methyl)-1-methyl-1H- | | | | | | |
| | imidazo[4,5-c]pyridine | | | | | | |
| 151 | 2-({Cis-4-[2-chloro-4- | 0.03 | C21H22ClF3N4 | 422.15 | 423.4 | 2 | I |
| | (trifluoromethyl)phenyl]- | | | | | | |
| | 3-methylpiperidin-1- | | | | | | |
| | yl}methyl)-1-methyl-1H- | | | | | | |
| | imidazo[4,5-b]pyridine | | | | | | |
| 152 | 2-({Cis-4-[2-chloro-4- | 0.0663 | C21H22ClF3N4 | 422.15 | 423.3 | 1.53 | I |
| | (trifluoromethyl)phenyl]- | | | | | | |
| | 3-methylpiperidin-1- | | | | | | |
| | yl}methyl)-1-methyl-1H- | | | | | | |
| | imidazo[4,5-c]pyridine | | | | | | |
| 153 | 2-({Cis-4-[2-fluoro-4- | 0.0273 | C21H22F4N4O | 422.17 | 423.4 | 1.9 | K |
| | (trifluoromethoxy)phenyl]- | | | | | | |
| | 3-methylpiperidin-1- | | | | | | |
| | yl}methyl)-1-methyl-1H- | | | | | | |
| | imidazo[4,5-b]pyridine, | | | | | | |
| | Eantiomer #1 | | | | | | |
| 154 | 2-({Cis-4-[2-fluoro-4- | 0.748 | C21H22F4N4O | 422.17 | 423.4 | 1.9 | K |
| | (trifluoromethoxy)phenyl]- | | | | | | |
| | 3-methylpiperidin-1- | | | | | | |
| | yl}methyl)-1-methyl-1H- | | | | | | |
| | imidazo[4,5-b]pyridine, | | | | | | |
| | Enantiomer #2 | | | | | | |
| 155 | 2-{[Cis-4-(2,4- | 0.0229 | C20H22Cl2N4 | 388.12 | 389.1 | 2.4 | I |
| | dichlorophenyl)-3- | | | | | | |
| | methylpiperidin-1- | | | | | | |
| | yl]methyl}-1-methyl-1H- | | | | | | |
| | imidazo[4,5-b]pyridine | | | | | | |
| 156 | 2-{[Cis-4-(2,4- | 0.0882 | C20H22Cl2N4 | 388.12 | 389.1 | 2 | I |
| | dichlorophenyl)-3- | | | | | | |
| | methylpiperidin-1- | | | | | | |
| | yl]methyl}-1-methyl-1H- | | | | | | |
| | imidazo[4,5-c]pyridine | | | | | | |
| 157 | 1-methyl-2-({Cis-3- | 0.277 | C21H23F3N4O | 404.18 | 405.1 | 2 | I |
| | methyl-4-[4- | | | | | | |
| | (trifluoromethoxy)phenyl]- | | | | | | |
| | piperidin-1- | | | | | | |
| | yl}methyl)-1H- | | | | | | |
| | imidazo[4,5-b]pyridine | | | | | | |
| 158 | 1-methyl-2-({Cis-3- | 0.262 | C21H23F3N4O | 404.18 | 405.1 | 1.8 | I |
| | methyl-4-[4- | | | | | | |
| | (trifluoromethoxy)phenyl]- | | | | | | |
| | piperidin-1- | | | | | | |
| | yl}methyl)-1H- | | | | | | |
| | imidazo[4,5-c]pyridine | | | | | | |
| 159 | 2-({Cis-4-[2-fluoro-4- | <0.0313 | C21H22F4N4O | 422.17 | 423.1 | 1.9 | K |
| | (trifluoromethoxy)phenyl]- | | | | | | |
| | 3-methylpiperidin-1- | | | | | | |
| | yl}methyl)-1-methyl-1H- | | | | | | |
| | imidazo[4,5-c]pyridine, | | | | | | |
| | enantiomer #2 | | | | | | |
| 160 | 2-{[Cis-4-(4-chloro-2- | 0.102 | C21H25ClN4 | 368.18 | 369.1 | 2.3 | I |
| | methylphenyl)-3- | | | | | | |
| | methylpiperidin-1- | | | | | | |
| | yl]methyl}-1-methyl-1H- | | | | | | |
| | imidazo[4,5-b]pyridine | | | | | | |
| 161 | 2-{[Cis-4-(4-chloro-2- | 0.393 | C21H25ClN4 | 368.18 | 369.2 | 1.8 | I |
| | methylphenyl)-3- | | | | | | |
| | methylpiperidin-1- | | | | | | |
| | yl]methyl}-1-methyl-1H- | | | | | | |
| | imidazo[4,5-c]pyridine | | | | | | |
| 162 | 2-({Cis-4-[2-chloro-4- | <0.0180 | C21H22ClF3N4 | 422.15 | 423.1 | 2.2 | K |
| | (trifluoromethyl)phenyl]- | | | | | | |
| | 3-methylpiperidin-1- | | | | | | |
| | yl}methyl)-1-methyl-1H- | | | | | | |
| | imidazo[4,5-b]pyridine, | | | | | | |
| | Enantiomer #1 | | | | | | |
| 163 | 2-({Cis-4-[2-chloro-4- | 0.396 | C21H22ClF3N4 | 422.15 | 423.1 | 2.2 | K |
| | (trifluoromethyl)phenyl]- | | | | | | |
| | 3-methylpiperidin-1- | | | | | | |
| | yl}methyl)-1-methyl-1H- | | | | | | |
| | imidazo[4,5-b]pyridine, | | | | | | |
| | Enantiomer #2 | | | | | | |
| 164 | 1-methyl-2-({cis-3- | <0.0743 | C22H25F3N4O | 418.2 | 419.2 | 2 | I |
| | methyl-4-[2-methyl-4- | | | | | | |
| | (trifluoromethoxy)phenyl]- | | | | | | |
| | piperidin-1- | | | | | | |
| | yl}methyl)-1H- | | | | | | |
| | imidazo[4,5-b]pyridine | | | | | | |
| 165 | 1-methyl-2-({cis-3- | 0.514 | C22H25F3N4O | 418.2 | 418.2 | 2.2 | I |
| | methyl-4-[2-methyl-4- | | | | | | |
| | (trifluoromethoxy)phenyl]- | | | | | | |
| | piperidin-1- | | | | | | |
| | yl}methyl)-1H- | | | | | | |
| | imidazo[4,5-c]pyridine | | | | | | |
| 166 | 2-{[Cis-4-(2-fluoro-4- | 0.0823 | C23H29FN4O | 396.23 | 397.2 | 1.9 | I |
| | isopropoxyphenyl)-3- | | | | | | |
| | methylpiperidin-1- | | | | | | |
| | yl]methyl}-1-methyl-1H- | | | | | | |
| | imidazo[4,5-b]pyridine | | | | | | |
| 167 | 2-{[Cis-4-(2-fluoro-4- | >0.172 | C23H29FN4O | 396.23 | 397.2 | 1.8 | I |
| | isopropoxyphenyl)-3- | | | | | | |
| | methylpiperidin-1- | | | | | | |
| | yl]methyl}-1-methyl-1H- | | | | | | |
| | imidazo[4,5-c]pyridine | | | | | | |
| 169 | 2-{[Cis-4-(2-chloro-4- | 0.174 | C23H29ClN4O | 412.2 | 413.4 | 1.91 | I |
| | isopropoxyphenyl)-3- | | | | | | |
| | methylpiperidin-1- | | | | | | |
| | yl]methyl}-1-methyl-1H- | | | | | | |
| | imidazo[4,5-b]pyridine | | | | | | |
| 170 | 2-{[Cis-4-(2-chloro-4- | 0.284 | C23H29ClN4O | 412.2 | 413.4 | 1.56 | I |
| | isopropoxyphenyl)-3- | | | | | | |
| | methylpiperidin-1- | | | | | | |
| | yl]methyl}-1-methyl-1H- | | | | | | |
| | imidazo[4,5-c]pyridine | | | | | | |
| 171 | 2-({Cis-4-[2-chloro-4- | 1.52 | C21H22ClF3N4 | 422.15 | 423.3 | 1.9 | K |
| | (trifluoromethyl)phenyl]- | | | | | | |
| | 3-methylpiperidin-1- | | | | | | |
| | yl}methyl)-1-methyl-1H- | | | | | | |
| | imidazo[4,5-c]pyridine, | | | | | | |
| | Enantiomer #1 | | | | | | |
| 172 | 2-({Cis-4-[2-chloro-4- | 0.0485 | C21H22ClF3N4 | 422.15 | 423.3 | 1.9 | K |
| | (trifluoromethyl)phenyl]- | | | | | | |
| | 3-methylpiperidin-1- | | | | | | |
| | yl}methyl)-1-methyl-1H- | | | | | | |
| | imidazo[4,5-c]pyridine, | | | | | | |
| | Enantiomer #2 | | | | | | |
| 173 | 2-({Cis-4-[4-methoxy-2- | 0.214 | C22H25F3N4O | 418.2 | 419.4 | 1.7 | I |
| | (trifluoromethyl)phenyl]- | | | | | | |
| | 3-methylpiperidin-1- | | | | | | |
| | yl}methyl)-1-methyl-1H- | | | | | | |
| | imidazo[4,5-b]pyridine | | | | | | |
| 174 | 2-({Cis-4-[4-methoxy-2- | >1.69 | C22H25F3N4O | 418.2 | 419.4 | 1.3 | I |
| | (trifluoromethyl)phenyl]- | | | | | | |
| | 3-methylpiperidin-1- | | | | | | |
| | yl}methyl)-1-methyl-1H- | | | | | | |
| | imidazo[4,5-c]pyridine | | | | | | |
| 175 | 1-methyl-2-({Cis-3- | >3.20 | C21H23F3N4O | 404.18 | 405.3 | 1.6 | K |
| | methyl-4-[4- | | | | | | |
| | (trifluoromethoxy)phenyl]- | | | | | | |
| | piperidin-1- | | | | | | |
| | yl}methyl)-1H- | | | | | | |
| | imidazo[4,5-c]pyridine, | | | | | | |
| | enantiomer #1 | | | | | | |
| 176 | 1-methyl-2-({Cis-3- | 0.0989 | C21H23F3N4O | 404.18 | 405.3 | 1.6 | K |
| | methyl-4-[4- | | | | | | |
| | (trifluoromethoxy)phenyl]- | | | | | | |
| | piperidin-1- | | | | | | |
| | yl}methyl)-1H- | | | | | | |
| | imidazo[4,5-c]pyridine, | | | | | | |
| | enantiomer #2 | | | | | | |
| 177 | 2-({Cis-4-[4- | 0.429 | C21H24F2N4O | 386.19 | 387.1 | 2.1 | I |
| | (difluoromethoxy)phenyl]- | | | | | | |
| | 3-methylpiperidin-1- | | | | | | |
| | yl}methyl)-1-methyl-1H- | | | | | | |
| | imidazo[4,5-b]pyridine | | | | | | |
| 178 | 2-({Cis-4-[4- | >1.35 | C21H24F2N4O | 386.19 | 387.1 | 2 | I |
| | (difluoromethoxy)phenyl]- | | | | | | |
| | 3-methylpiperidin-1- | | | | | | |
| | yl}methyl)-1-methyl-1H- | | | | | | |
| | imidazo[4,5-c]pyridine | | | | | | |
| 179 | 1-methyl-2-{[Cis-3- | 0.381 | C20H21F3N4 | 374.17 | 375.3 | 1.6 | I |
| | methyl-4-(2,4,5- | | | | | | |
| | trifluorophenyl)piperidin- | | | | | | |
| | 1-yl]methyl}-1H- | | | | | | |
| | imidazo[4,5-b]pyridine | | | | | | |
| 180 | 1-methyl-2-{[Cis-3- | 0.762 | C20H21F3N4 | 374.17 | 375.4 | 0.9 | I |
| | methyl-4-(2,4,5- | | | | | | |
| | trifluorophenyl)piperidin- | | | | | | |
| | 1-yl]methyl}-1H- | | | | | | |
| | imidazo[4,5-c]pyridine | | | | | | |
| 181 | 2-{[Cis-4-(3-fluoro-4- | 1.09 | C23H29FN4O | 396.23 | 397.1 | 1.6 | I |
| | isopropoxyphenyl)-3- | | | | | | |
| | methylpiperidin-1- | | | | | | |
| | yl]methyl}-1-methyl-1H- | | | | | | |
| | imidazo[4,5-c]pyridine | | | | | | |
| 182 | 2-{[Cis-4-(3-fluoro-4- | 0.577 | C23H29FN4O | 396.23 | 397.1 | 1.9 | I |
| | isopropoxyphenyl)-3- | | | | | | |
| | methylpiperidin-1- | | | | | | |
| | yl]methyl}-1-methyl-1H- | | | | | | |
| | imidazo[4,5-b]pyridine | | | | | | |
| 183 | 2-({Cis-4-[3-fluoro-4- | 0.0532 | C21H22F4N4 | 406.18 | 407 | 2 | K |
| | (trifluoromethyl)phenyl]- | | | | | | |
| | 3-methylpiperidin-1- | | | | | | |
| | yl}methyl)-1-methyl-1H- | | | | | | |
| | imidazo[4,5-c]pyridine, | | | | | | |
| | Enantiomer #2 | | | | | | |
| 184 | 2-({Cis-4-[3-fluoro-4- | >2.71 | C21H22F4N4 | 406.18 | 407 | 2 | K |
| | (trifluoromethyl)phenyl]- | | | | | | |
| | 3-methylpiperidin-1- | | | | | | |
| | yl}methyl)-1-methyl-1H- | | | | | | |
| | imidazo[4,5-c]pyridine, | | | | | | |
| | Enantiomer #1 | | | | | | |
| 185 | 2-{[Cis-4-(2,4- | 0.0642 | C20H22Cl2N4 | 388.12 | 389 | 2.2 | K |
| | dichlorophenyl)-3- | | | | | | |
| | methylpiperidin-1- | | | | | | |
| | yl]methyl}-1-methyl-1H- | | | | | | |
| | imidazo[4,5-b]pyridine, | | | | | | |
| | enantiomer #1 | | | | | | |
| 186 | 2-{[Cis-4-(2,4- | 0.611 | C20H22Cl2N4 | 388.12 | 389 | 2.2 | K |
| | dichlorophenyl)-3- | | | | | | |
| | methylpiperidin-1- | | | | | | |
| | yl]methyl}-1-methyl-1H- | | | | | | |
| | imidazo[4,5-b]pyridine, | | | | | | |
| | enantiomer #2 | | | | | | |
| 187 | 2-({Cis-4-[2-chloro-4- | 0.063 | C21H22ClF3N4O | 438.14 | 438.9 | 2.2 | I |
| | (trifluoromethoxy)phenyl]- | | | | | | |
| | 3-methylpiperidin-1- | | | | | | |
| | yl}methyl)-1-methyl-1H- | | | | | | |
| | imidazo[4,5-b]pyridine | | | | | | |
| 188 | 2-({Cis-4-[2-chloro-4- | 0.194 | C21H22ClF3N4O | 438.14 | 438.9 | 2.1 | I |
| | (trifluoromethoxy)phenyl]- | | | | | | |
| | 3-methylpiperidin-1- | | | | | | |
| | yl}methyl)-1-methyl-1H- | | | | | | |
| | imidazo[4,5-c]pyridine | | | | | | |
| 189 | 1-methyl-2-{[Cis-3- | 0.621 | C24H30N4 | 374.25 | 375.1 | 2.2 | I |
| | methyl-4-(5,6,7,8- | | | | | | |
| | tetrahydronaphthalen- | | | | | | |
| | 2-yl)piperidin-1- | | | | | | |
| | yl]methyl}-1H- | | | | | | |
| | imidazo[4,5-b]pyridine | | | | | | |
| 190 | 1-methyl-2-{[Cis-3- | 0.573 | C24H30N4 | 374.25 | 375.1 | 1.9 | I |
| | methyl-4-(5,6,7,8- | | | | | | |
| | tetrahydronaphthalen- | | | | | | |
| | 2-yl)piperidin-1- | | | | | | |
| | yl]methyl}-1H- | | | | | | |
| | imidazo[4,5-c]pyridine | | | | | | |
| 191 | 2-{[Cis-4-(4-fluoro-2- | 0.61 | C23H29FN4O | 396.23 | 397.1 | 2.2 | I |
| | isopropoxyphenyl)-3- | | | | | | |
| | methylpiperidin-1- | | | | | | |
| | yl]methyl}-1-methyl-1H- | | | | | | |
| | imidazo[4,5-b]pyridine | | | | | | |
| 192 | 2-{[Cis-4-(4-fluoro-2- | 1.17 | C23H29FN4O | 396.23 | 397.1 | 2.1 | I |
| | isopropoxyphenyl)-3- | | | | | | |
| | methylpiperidin-1- | | | | | | |
| | yl]methyl}-1-methyl-1H- | | | | | | |
| | imidazo[4,5-c]pyridine | | | | | | |
| 193 | 2-{[Cis-4-(3-chloro-4- | 0.59 | C23H29ClN4O | 412.2 | 413.1 | 1.9 | I |
| | isopropoxyphenyl)-3- | | | | | | |
| | methylpiperidin-1- | | | | | | |
| | yl]methyl}-1-methyl-1H- | | | | | | |
| | imidazo[4,5-c]pyridine | | | | | | |
| 194 | 2-{[Cis-4-(4-ethoxy-3- | >5.36 | C22H27FN4O | 382.22 | 383.2 | 2 | I |
| | fluorophenyl)-3- | | | | | | |
| | methylpiperidin-1- | | | | | | |
| | yl]methyl}-1-methyl-1H- | | | | | | |
| | imidazo[4,5-c]pyridine | | | | | | |
| 195 | 2-{[Cis-4-(3-chloro-4- | 0.506 | C23H29ClN4O | 412.2 | 413.1 | 2 | I |
| | isopropoxyphenyl)-3- | | | | | | |
| | methylpiperidin-1- | | | | | | |
| | yl]methyl}-1-methyl-1H- | | | | | | |
| | imidazo[4,5-b]pyridine | | | | | | |
| 196 | 2-({Cis-4-[2-fluoro-4- | 0.056 | C21H22F4N4O | 422.17 | 423.3 | 2 | K |
| | (trifluoromethoxy)phenyl]- | | | | | | |
| | 3-methylpiperidin-1- | | | | | | |
| | yl}methyl)-1-methyl-1H- | | | | | | |
| | imidazo[4,5-c]pyridine, | | | | | | |
| | enantiomer #2 | | | | | | |
| 197 | 2-({Cis-4-[4-fluoro-3- | 0.128 | C21H22F4N4 | 406.18 | 407.3 | 1.8 | I |
| | (trifluoromethyl)phenyl]- | | | | | | |
| | 3-methylpiperidin-1- | | | | | | |
| | yl}methyl)-1-methyl-1H- | | | | | | |
| | imidazo[4,5-b]pyridine | | | | | | |
| 198 | 2-({Cis-4-[4-fluoro-3- | 0.176 | C21H22F4N4 | 406.18 | 407.3 | 1.1 | I |
| | (trifluoromethyl)phenyl]- | | | | | | |
| | 3-methylpiperidin-1- | | | | | | |
| | yl}methyl)-1-methyl-1H- | | | | | | |
| | imidazo[4,5-c]pyridine | | | | | | |
| 199 | 2-({Cis-4-[4- | 0.122 | C21H23F3N4O | 404.18 | 405.2 | | I |
| | (difluoromethoxy)-2- | | | | | | |
| | fluorophenyl]-3- | | | | | | |
| | methylpiperidin-1- | | | | | | |
| | yl}methyl)-1-methyl-1H- | | | | | | |
| | imidazo[4,5-c]pyridine | | | | | | |
| 200 | 2-{[Cis-4-(2,3-dihydro- | 0.469 | C22H26N4O | 362.21 | 363.4 | 1.1 | I |
| | 1-benzofuran-5-yl)-3- | | | | | | |
| | methylpiperidin-1- | | | | | | |
| | yl]methyl}-1-methyl-1H- | | | | | | |
| | imidazo[4,5-b]pyridine | | | | | | |
| 201 | 2-{[Cis-4-(2,3-dihydro- | >1.07 | C22H26N4O | 362.21 | 363.4 | 0.5 | I |
| | 1-benzofuran-5-yl)-3- | | | | | | |
| | methylpiperidin-1- | | | | | | |
| | yl]methyl}-1-methyl-1H- | | | | | | |
| | imidazo[4,5-c]pyridine | | | | | | |
| 202 | 2-({Cis-4-[2-chloro-4- | 0.125 | C21H23ClF2N4O | 420.15 | 421.1 | | I |
| | (difluoromethoxy)phenyl]- | | | | | | |
| | 3-methylpiperidin-1- | | | | | | |
| | yl}methyl)-1-methyl-1H- | | | | | | |
| | imidazo[4,5-c]pyridine | | | | | | |
| 203 | 2-({Cis-4-[2-chloro-4- | 0.0436 | C21H23ClF2N4O | 420.15 | 421.1 | | I |
| | (difluoromethoxy)phenyl]- | | | | | | |
| | 3-methylpiperidin-1- | | | | | | |
| | yl}methyl)-1-methyl-1H- | | | | | | |
| | imidazo[4,5-b]pyridine | | | | | | |
| 204 | 2-({Cis-4-[4-fluoro-2- | 0.0283 | C21H22F4N4 | 406.18 | 407.1 | 2.1 | I |
| | (trifluoromethyl)phenyl]- | | | | | | |
| | 3-methylpiperidin-1- | | | | | | |
| | yl}methyl)-1-methyl-1H- | | | | | | |
| | imidazo[4,5-b]pyridine | | | | | | |
| 205 | 2-{[Cis-4-(4-fluoro-2- | 0.243 | C21H25FN4 | 352.21 | 353.3 | 0.9 | I |
| | methylphenyl)-3- | | | | | | |
| | methylpiperidin-1- | | | | | | |
| | yl]methyl}-1-methyl-1H- | | | | | | |
| | imidazo[4,5-c]pyridine | | | | | | |
| 206 | 2-{[Cis-4-(4-fluoro-2- | 0.0886 | C21H25FN4 | 352.21 | 353.3 | | I |
| | methylphenyl)-3- | | | | | | |
| | methylpiperidin-1- | | | | | | |
| | yl]methyl}-1-methyl-1H- | | | | | | |
| | imidazo[4,5-b]pyridine | | | | | | |
| 207 | 2-({Cis-4-[4-chloro-2- | 0.108 | C21H22ClF3N4 | 422.15 | 423.2 | 2.1 | I |
| | (trifluoromethyl)phenyl]- | | | | | | |
| | 3-methylpiperidin-1- | | | | | | |
| | yl}methyl)-1-methyl-1H- | | | | | | |
| | imidazo[4,5-b]pyridine | | | | | | |
| 208 | 2-({Cis-4-[4-chloro-2- | 0.11 | C21H22ClF3N4 | 422.15 | 423.2 | 1.6 | I |
| | (trifluoromethyl)phenyl]- | | | | | | |
| | 3-methylpiperidin-1- | | | | | | |
| | yl}methyl)-1-methyl-1H- | | | | | | |
| | imidazo[4,5-c]pyridine | | | | | | |
| 209 | 2-{[Cis-4-(2-chloro-4- | 0.159 | C21H25ClN4 | 368.91 | 369.3 | 1.3 | I |
| | methylphenyl)-3- | | | | | | |
| | methylpiperidin-1- | | | | | | |
| | yl]methyl}-1-methyl-1H- | | | | | | |
| | imidazo[4,5-c]pyridine | | | | | | |
| 210 | 2-{[Cis-4-(2-chloro-4- | 0.0626 | C21H25ClN4 | 368.91 | 369.3 | 1.9 | I |
| | methylphenyl)-3- | | | | | | |
| | methylpiperidin-1- | | | | | | |
| | yl]methyl}-1-methyl-1H- | | | | | | |
| | imidazo[4,5-b]pyridine | | | | | | |
| 211 | 2-({Cis-3-methoxy-4-[4- | 0.478 | C21H23F3N4O | 404.18 | 405.3 | 1.6 | I |
| | (trifluoromethyl)phenyl]- | | | | | | |
| | piperidin-1-yl}methyl)- | | | | | | |
| | 1-methyl-1H- | | | | | | |
| | imidizo[4,5-b]pyridine | | | | | | |
| 212 | 2-({Cis-4-[3-chloro-4- | 0.209 | C21H22ClF3N4O | 438.14 | 439 | 2.2 | I |
| | (trifluoromethoxy)phenyl]- | | | | | | |
| | 3-methylpiperidin-1- | | | | | | |
| | yl}methyl)-1-methyl-1H- | | | | | | |
| | imidazo[4,5-c]pyridine | | | | | | |
| 213 | 2-({Cis-4-[3-chloro-4- | 0.253 | C21H22ClF3N4O | 438.14 | 439 | 2.5 | I |
| | (trifluoromethoxy)phenyl]- | | | | | | |
| | 3-methylpiperidin-1- | | | | | | |
| | yl}methyl)-1-methyl-1H- | | | | | | |
| | imidazo[4,5-b]pyridine | | | | | | |
| 214 | 2-({Cis-4-[2-methoxy-4- | 0.124 | C22H25F3N4O2 | 434.19 | 435.3 | 1.8 | I |
| | (trifluoromethoxy)phenyl]- | | | | | | |
| | 3-methylpiperidin-1- | | | | | | |
| | yl}methyl)-1-methyl-1H- | | | | | | |
| | imidazo[4,5-c]pyridine | | | | | | |
| 215 | 2({Cis-4-[2-methoxy-4- | 0.0594 | C22H25F3N4O2 | 434.19 | 435.3 | 2 | I |
| | (trifluoromethoxy)phenyl]- | | | | | | |
| | 3-methylpiperidin-1- | | | | | | |
| | yl}methyl)-1-methyl-1H- | | | | | | |
| | imidazo[4,5-b]pyridine | | | | | | |
| 216 | 2-({Cis-4-[4- | 0.287 | C24H29F3N4O | 446.23 | 447.4 | 1.7 | I |
| | isopropoxy-2- | | | | | | |
| | (trifluoromethyl)phenyl]- | | | | | | |
| | 3-methylpiperidin-1- | | | | | | |
| | yl}methyl)-1-methyl-1H- | | | | | | |
| | imidazo[4,5-c]pyridine | | | | | | |
| 217 | 2-{[Cis-4-(2-fluoro-4- | 0.0298 | C23H29FN4O | 396.23 | 397.3 | 1.8 | K |
| | isopropoxyphenyl)-3- | | | | | | |
| | methylpiperidin-1- | | | | | | |
| | yl]methyl}-1-methyl-1H- | | | | | | |
| | imidazo[4,5-b]pyridine, | | | | | | |
| | enantiomer #1 | | | | | | |
| 218 | 2-{[Cis-4-(2-fluoro-4- | >0.909 | C23H29FN4O | 396.23 | 397.3 | 1.8 | K |
| | isopropoxyphenyl)-3- | | | | | | |
| | methylpiperidin-1- | | | | | | |
| | yl]methyl}-1-methyl-1H- | | | | | | |
| | imidazo[4,5-b]pyridine, | | | | | | |
| | enantiomer #2 | | | | | | |
| 219 | 2-{[Cis-4-(2-fluoro-4- | 0.0606 | C23H29FN4O | 396.23 | 397.3 | 1.3 | K |
| | isopropoxyphenyl)-3- | | | | | | |
| | methylpiperidin-1- | | | | | | |
| | yl]methyl}-1-methyl-1H- | | | | | | |
| | imidazo[4,5-c]pyridine, | | | | | | |
| | enantiomer #1 | | | | | | |
| 220 | 2-{[Cis-4-(2-fluoro-4- | >1.19 | C23H29FN4O | 396.23 | 397.3 | 1.3 | K |
| | isopropoxyphenyl)-3- | | | | | | |
| | methylpiperidin-1- | | | | | | |
| | yl]methyl}-1-methyl-1H- | | | | | | |
| | imidazo[4,5-c]pyridine, | | | | | | |
| | enantiomer #2 | | | | | | |
| 221 | 2-{[Cis-4-(2-chloro-4- | 0.0778 | C23H29ClN4O | 412.2 | 413.3 | 1.9 | K |
| | isopropoxyphenyl)-3- | | | | | | |
| | methylpiperidin-1- | | | | | | |
| | yl]methyl}-1-methyl-1H- | | | | | | |
| | imidazo[4,5-b]pyridine, | | | | | | |
| | enantiomer #1 | | | | | | |
| 222 | 2-{[Cis-4-(2-chloro-4- | >0.603 | C23H29ClN4O | 412.2 | 413.3 | 1.9 | K |
| | isopropoxyphenyl)-3- | | | | | | |
| | methylpiperidin-1- | | | | | | |
| | yl]methyl)-1-methyl-1H- | | | | | | |
| | imidazo[4,5-b]pyridine, | | | | | | |
| | enantiomer # 2 | | | | | | |
| 223 | 2-({Cis-4-[4- | >0.129 | C21H24F2N4O | 386.19 | 387.3 | 1.6 | K |
| | (difluoromethoxy)phenyl]- | | | | | | |
| | 3-methylpiperidin-1- | | | | | | |
| | yl}methyl)-1-methyl-1H- | | | | | | |
| | imidazo[4,5-b]pyridine, | | | | | | |
| | enantiomer #2 | | | | | | |
| 224 | 2-({Cis-4-[4- | >1.92 | C21H24F2N4O | 386.19 | 387.3 | 1.6 | K |
| | (difluoromethoxy)phenyl]- | | | | | | |
| | 3-methylpiperidin-1- | | | | | | |
| | yl}methyl)-1-methyl-1H- | | | | | | |
| | imidazo[4,5-b]pyridine, | | | | | | |
| | enantiomer #1 | | | | | | |
| 225 | 1-methyl-2-{[Cis-3- | 0.095 | C20H21F3N4 | 374.17 | 375.3 | 1.1 | K |
| | methyl-4-(2,4,5- | | | | | | |
| | trifluorophenyl)piperidin- | | | | | | |
| | 1-yl]methyl}-1H- | | | | | | |
| | imidazo[4,5-c]pyridine, | | | | | | |
| | enantiomer #2 | | | | | | |
| 226 | 1-methyl-2-{[Cis-3- | >1.26 | C20H21F3N4 | 374.17 | 375.3 | 1.1 | K |
| | methyl-4-(2,4,5- | | | | | | |
| | trifluorophenyl)piperidin- | | | | | | |
| | 1-yl]methyl}-1H- | | | | | | |
| | imidazo[4,5-c]pyridine, | | | | | | |
| | enantiomer #1 | | | | | | |
| 227 | 2-({Cis-3-methoxy-4-[4- | 0.606 | C21H23F3N4O | 404.43 | 405.2 | | I |
| | (trifluoromethyl)phenyl]- | | | | | | |
| | piperidin-1-yl}methyl)- | | | | | | |
| | 1-methyl-1H- | | | | | | |
| | imidazo[4,5-c]pyridine | | | | | | |
| 228 | 2-({4-[2-fluoro-6- | >0.458 | C20H20F4N4 | 392.16 | 393.3 | 0.9 | I |
| | (trifluoromethyl)phenyl]- | | | | | | |
| | piperidin-1-yl}methyl)- | | | | | | |
| | 1-methyl-1H- | | | | | | |
| | imidazo[4,5-c]pyridine | | | | | | |
| 229 | 2-({4-[2-fluoro-6- | 0.212 | C20H20F4N4 | 392.16 | 393.3 | 1.6 | I |
| | (trifluoromethyl)phenyl]- | | | | | | |
| | piperidin-1-yl}methyl)- | | | | | | |
| | 1-methyl-1H- | | | | | | |
| | imidazo[4,5-b]pyridine | | | | | | |
| 230 | 2-({4-[2-fluoro-4- | 0.0249 | C20H20F4N4O | 404.18 | 405.3 | 1.1 | I |
| | (trifluoromethoxy)phenyl]- | | | | | | |
| | piperidin-1- | | | | | | |
| | yl}methyl)-1-methyl-1H- | | | | | | |
| | imidazo[4,5-b]pyridine | | | | | | |
| 231 | 1-methyl-2-({4-[4- | 0.0845 | C20H21F3N4O | 390.17 | 391 | 1.8 | I |
| | (trifluoromethoxy)phenyl]- | | | | | | |
| | piperidin-1- | | | | | | |
| | yl}methyl)-1H- | | | | | | |
| | imidazo[4,5-b]pyridine | | | | | | |
| 232 | 2-{[Cis-4-(2.4- | 0.731 | C20H22Cl2N4 | 388.12 | 389.2 | 1.7 | K |
| | dichlorophenyl)-3- | | | | | | |
| | methylpiperidin-1- | | | | | | |
| | yl]methyl}-1-methyl-1H- | | | | | | |
| | imidazo[4,5-c]pyridine, | | | | | | |
| | enantiomer #1 | | | | | | |
| 233 | 2-{[Cis-4-(2.4- | 0.0723 | C20H22Cl2N4 | 389.33 | 389.2 | 1.7 | K |
| | dichlorophenyl)-3- | | | | | | |
| | methylpiperidin-1- | | | | | | |
| | yl]methyl}-1-methyl-1H- | | | | | | |
| | imidazo[4,5-c]pyridine, | | | | | | |
| | enantiomer #2 | | | | | | |
| 234 | 2-({Cis-4-[3-chloro-4- | 0.101 | C21H22ClF3N4 | 422.15 | 423.3 | 2 | I |
| | (trifluoromethyl)phenyl]- | | | | | | |
| | 3-methylpiperidin-1- | | | | | | |
| | yl}methyl)-1-methyl-1H- | | | | | | |
| | imidazo[4,5-b]pyridine | | | | | | |
| 235 | 2-({Cis-4-[3-chloro-4- | 0.135 | C21H22ClF3N4 | 422.15 | 423.3 | 1.8 | I |
| | (trifluoromethyl)phenyl]- | | | | | | |
| | 3-methylpiperidin-1 | | | | | | |
| | yl}methyl)-1-methyl-1H- | | | | | | |
| | imidazo[4,5-c]pyridine | | | | | | |
| 236 | 2-({4-[2-fluoro-4- | 0.0508 | C20H20F4N4 | 392.16 | 393 | 1.8 | I |
| | (trifluoromethyl)phenyl]- | | | | | | |
| | piperidin-1-yl}methyl)- | | | | | | |
| | 1-methyl-1H- | | | | | | |
| | imidazo[4,5-c]pyridine | | | | | | |
| 237 | 1-methyl-2({4-[4- | 0.16 | C20H21F3N4O | 390.17 | 391 | 1 | I |
| | (trifluoromethoxy)phenyl]- | | | | | | |
| | piperidin-1- | | | | | | |
| | yl}methyl)-1H- | | | | | | |
| | imidazo[4,5-c]pyridine | | | | | | |
| 238 | 1-methyl-2-({Cis-3- | 0.192 | C22H25F3N4 | 402.2 | 403.3 | 1.3 | I |
| | methyl-4-[2-methyl-4- | | | | | | |
| | (trifluoromethyl)phenyl]- | | | | | | |
| | piperidin-1-yl}methyl)- | | | | | | |
| | 1H-imdazo[4,5- | | | | | | |
| | c]pyridine | | | | | | |
| 239 | 1-methyl-2-({Cis-3- | 0.088 | C22H25F3N4 | 402.2 | 403.3 | 1.9 | I |
| | methyl-4-[2-methyl-4- | | | | | | |
| | (trifluoromethyl)phenyl]- | | | | | | |
| | piperidin-1-yl}methyl)- | | | | | | |
| | 1H-imidazo[4,5- | | | | | | |
| | b]pyridine | | | | | | |
| 240 | 1-methyl-2-({4-[2- | 0.0968 | C21H23F3N4O | 404.18 | 405.1 | | I |
| | methyl-4- | | | | | | |
| | (trifluoromethoxy)phenyl]- | | | | | | |
| | piperidin-1- | | | | | | |
| | yl}methyl)-1H- | | | | | | |
| | imidazo[4,5-c]pyridine | | | | | | |
| 241 | 1-methyl-2-({4-[2- | 0.0507 | C21H23F3N4O | 404.18 | 405.1 | | I |
| | methyl-4- | | | | | | |
| | (trifluoromethoxy)phenyl]- | | | | | | |
| | piperidin-1- | | | | | | |
| | yl}methyl)-1H- | | | | | | |
| | imidazo[4,5-b]pyridine | | | | | | |
| 242 | 1-methyl-2-({4-[2- | 0.0858 | C21H23F3N4 | 388.19 | 389.3 | 1.5 | I |
| | methyl-4- | | | | | | |
| | (trifluoromethyl)phenyl]- | | | | | | |
| | piperidin-1-yl}methyl)- | | | | | | |
| | 1H-imidazo[4,5- | | | | | | |
| | c]pyridine | | | | | | |
| 243 | 1-methyl-2-({4-[2- | 0.0511 | C21H23F3N4 | 388.19 | 389.3 | 1.7 | I |
| | methyl-4- | | | | | | |
| | (trifluoromethyl)phenyl]- | | | | | | |
| | piperidin-1-yl}methyl)- | | | | | | |
| | 1H-imidazo[4,5- | | | | | | |
| | b]pyridine | | | | | | |
| 244 | 2-({Cis-4-[2-chloro-4- | 0.036 | C21H23ClF2N4O | 420.15 | 421.1 | 4.6 (chiral) | K |
| | (difluoromethoxy)phenyl]- | | | | | | |
| | 3-methylpiperidin-1- | | | | | | |
| | yl}methyl)-1-methyl-1H- | | | | | | |
| | imidazo[4,5-b]pyridine, | | | | | | |
| | enantiomer #1 | | | | | | |
| 245 | 2-({Cis-4-[2-chloro-4- | 0.262 | C21H23ClF2N4O | 420.15 | 421.1 | 7.2 (Chiral) | K |
| | (difluoromethoxy)phenyl]- | | | | | | |
| | 3-methylpiperidin-1- | | | | | | |
| | yl}methyl)-1-methyl-1H- | | | | | | |
| | imidazo[4,5-b]pyridine, | | | | | | |
| | enantiomer #2 | | | | | | |
| 246 | 2-({Cis-4-[2-chloro-4- | 0.0999 | C21H23ClF2N4O | 420.15 | 421.1 | 6.7 (chiral) | K |
| | (difluoromethoxy)phenyl]- | | | | | | |
| | 3-methylpiperidin-1- | | | | | | |
| | yl}methyl)-1-methyl-1H- | | | | | | |
| | imidazo[4,5-c]pyridine, | | | | | | |
| | enantiomer #1 | | | | | | |
| 247 | 2-({Cis-4-[2-chloro-4- | >1.41 | C21H23ClF2N4O | 420.15 | 421.1 | 8.6 (chiral) | K |
| | (difluoromethoxy)phenyl]- | | | | | | |
| | 3-methylpiperidin-1- | | | | | | |
| | yl}methyl)-1-methyl-1H- | | | | | | |
| | imidazo[4,5-c]pyridine, | | | | | | |
| | enantiomer #2 | | | | | | |
| 249 | 1-methyl-2-{[Cis-3- | 0.113 | C20H21F3N4 | 374.17 | 375.3 | 1.6 | I |
| | methyl-4-(2,3,4- | | | | | | |
| | trifluorophenyl)piperidin- | | | | | | |
| | 1-yl]methyl}-1H- | | | | | | |
| | imidazo[4,5-b]pyridine | | | | | | |
| 250 | 1-methyl-2-{[Cis-3- | >0.221 | C20H21F3N4 | 374.17 | 375.3 | 0.9 | I |
| | methyl-4-(2,3,4- | | | | | | |
| | trifluorophenyl)piperidin- | | | | | | |
| | 1-yl]methyl}-1H- | | | | | | |
| | imidazo[4,5-c]pyridine | | | | | | |
| 251 | 2-({Cis-4-[4-fluoro-2- | 0.021 | C21H22F4N4 | 406.18 | 406.9 | 2 | K |
| | (trifluoromethyl)phenyl]- | | | | | | |
| | 3-methylpiperidin-1- | | | | | | |
| | yl}methyl)-1-methyl-1H- | | | | | | |
| | imidazo[4,5-b]pyridine, | | | | | | |
| | enantiomer #1 | | | | | | |
| 252 | 2-({Cis-4-[4-fluoro-2- | 0.288 | C21H22F4N4 | 406.18 | 406.9 | 1.9 | K |
| | (trifluoromethyl)phenyl]- | | | | | | |
| | 3-methylpiperidin-1- | | | | | | |
| | yl}methyl)-1-methyl-1H- | | | | | | |
| | imidazo[4,5-b]pyridine, | | | | | | |
| | enantiomer #2 | | | | | | |
| 253 | 2-({Cis-4-[2-chloro-4- | 1.82 | C21H22ClF3N4O | 438.14 | 439.2 | 1.9 | K |
| | (trifluoromethoxy)phenyl]- | | | | | | |
| | 3-methylpiperidin-1- | | | | | | |
| | yl}methyl)-1-methyl-1H- | | | | | | |
| | imidazo[4,5-c]pyridine, | | | | | | |
| | enantiomer #1 | | | | | | |
| 254 | 2-({Cis-4-[2-chloro-4- | 0.0866 | C21H22ClF3N4O | 438.14 | 439.2 | 1.9 | K |
| | (trifluoromethoxy)phenyl]- | | | | | | |
| | 3-methylpiperidin-1- | | | | | | |
| | yl}methyl)-1-methyl-1H- | | | | | | |
| | imidazo[4,5-c]pyridine, | | | | | | |
| | enantiomer #2 | | | | | | |
| 255 | 2-({Cis-4-[2-chloro-4- | 0.0769 | C21H22ClF3N4O | 438.14 | 439.2 | 2 | K |
| | (trifluoromethoxy)phenyl]- | | | | | | |
| | 3-methylpiperidin-1- | | | | | | |
| | yl}methyl)-1-methyl-1H- | | | | | | |
| | imidazo[4,5-b]pyridine, | | | | | | |
| | enantiomer #1 | | | | | | |
| 256 | 2-({Cis-4-[2-chloro-4- | >0.704 | C21H22ClF3N4O | 438.14 | 439.2 | 2 | K |
| | (trifluoromethoxy)phenyl]- | | | | | | |
| | 3-methylpiperidin-1- | | | | | | |
| | yl}methyl)-1-methyl-1H- | | | | | | |
| | imidazo[4,5-b]pyridine, | | | | | | |
| | enantiomer #2 | | | | | | |
| 257 | 2-({Cis-4-[4- | 0.0677 | C21H23F3N4O | 404.18 | 405.1 | | I |
| | (difluoromethoxy)-2- | | | | | | |
| | fluorophenyl]-3- | | | | | | |
| | methylpiperidin-1- | | | | | | |
| | yl}methyl)-1-methyl-1H- | | | | | | |
| | imidazo[4,5-b]pyridine | | | | | | |
| 258 | 2-({Cis-4-[4-fluoro-2- | 0.125 | C21H22F4N4 | 406.18 | 407 | 1.9 | I |
| | (trifluoromethyl)phenyl]- | | | | | | |
| | 3-methylpiperidin-1- | | | | | | |
| | yl}methyl)-1-methyl-1H- | | | | | | |
| | imidazo[4,5-c]pyridine | | | | | | |
| 259 | 2-{[Cis-4-(4-fluoro-2- | 1.46 | C21H25FN4O | 368.2 | 369.3 | 1 | K |
| | methoxyphenyl)-3- | | | | | | |
| | methylpiperidin-1- | | | | | | |
| | yl]methyl}-1-methyl-1H- | | | | | | |
| | imidazo[4,5-c]pyridine, | | | | | | |
| | enantiomer #1 | | | | | | |
| 260 | 2-{[Cis-4-(4-fluoro-2- | 0.125 | C21H25FN4O | 368.2 | 369.3 | 1 | K |
| | methoxyphenyl)-3- | | | | | | |
| | methylpiperidin-1- | | | | | | |
| | yl]methyl}-1-methyl-1H- | | | | | | |
| | imidazo[4,5-c]pyridine, | | | | | | |
| | enantiomer #2 | | | | | | |
| 261 | 2-{[4-(2-fluoro-4- | 0.125 | C22H27FN4O | 382.22 | 383.3 | 1.3 | I |
| | isopropoxyphenyl)piperidin- | | | | | | |
| | 1-yl]methyl}-1- | | | | | | |
| | methyl-1H-imidazo[4,5- | | | | | | |
| | c]pyridine | | | | | | |
| 262 | 2-{[4-(2-fluoro-4- | 0.127 | C22H27FN4O | 382.22 | 383.3 | 1.6 | I |
| | isopropoxyphenyl)piperidin- | | | | | | |
| | 1-yl]methyl}-1- | | | | | | |
| | methyl-1H-imidazo[4,5- | | | | | | |
| | b]pyridine | | | | | | |
| 263 | 2-({4-[4- | 0.906 | C20H22F2N4O | 372.18 | 373.3 | 0.4 | I |
| | (difluoromethoxy)phenyl]- | | | | | | |
| | piperidin-1-yl}methyl)- | | | | | | |
| | 1-methyl-1H- | | | | | | |
| | imidazo[4,5-c]pyridine | | | | | | |
| 264 | 2-({4-[4- | 0.554 | C20H22F2N4O | 372.18 | 373 | 0.9 | I |
| | (difluoromethoxy)phenyl]- | | | | | | |
| | piperidin-1-yl}methyl)- | | | | | | |
| | 1-methyl-1H- | | | | | | |
| | imidazo[4,5-b]pyridine | | | | | | |
| 265 | 2-({4-[2-chloro-4- | 0.164 | C20H21ClF2N4O | 406.14 | 406.9 | 1.7 | I |
| | (difluoromethoxy)phenyl]- | | | | | | |
| | piperidin-1-yl}methyl)- | | | | | | |
| | 1-methyl-1H- | | | | | | |
| | imidazo[4,5-c]pyridine | | | | | | |
| 266 | 2-({4-[2-chloro-4- | 0.0549 | C20H21ClF2N4O | 406.14 | 407 | 1.9 | I |
| | (difluoromethoxy)phenyl]- | | | | | | |
| | piperidin-1-yl}methyl)- | | | | | | |
| | 1-methyl-1H- | | | | | | |
| | imidazo[4,5-b]pyridine | | | | | | |
| 267 | 2-{[4-(2-chloro-4- | 0.0855 | C22H27ClN4O | 398.19 | 399 | 1.7 | I |
| | isopropoxyphenyl)piperidin- | | | | | | |
| | 1-yl]methyl}-1- | | | | | | |
| | methyl-1H-imidazo[4,5- | | | | | | |
| | c]pyridine | | | | | | |
| 268 | 2-({4-[2-fluoro-4- | 0.0127 | C20H20F4N4 | 392.16 | 393 | 2 | I |
| | (trifluoromethyl)phenyl]- | | | | | | |
| | piperidin-1-yl}methyl)- | | | | | | |
| | 1-methyl-1H- | | | | | | |
| | imidazo[4,5-b]pyridine | | | | | | |
| 269 | 2-{[4-(2-chloro-4- | 0.0657 | C22H27ClN4O | 398.19 | 399 | 1.8 | I |
| | isopropoxyphenyl)piperidin- | | | | | | |
| | 1-yl]methyl}-1- | | | | | | |
| | methyl-1H-imidazo[4,5- | | | | | | |
| | b]pyridine | | | | | | |
| 270 | 2-({4-[4-methoxy-2- | 0.494 | C21H23F3N4O | 404.18 | 405 | 1.4 | I |
| | (trifluoromethyl)phenyl]- | | | | | | |
| | piperidin-1-yl}methyl)- | | | | | | |
| | 1-methyl-1H- | | | | | | |
| | imidazo[4,5-c]pyridine | | | | | | |
| 271 | 2-({4-[4-methoxy-2- | 0.494 | C21H23F3N4O | 404.18 | 405 | 1.7 | I |
| | (trifluoromethyl)phenyl]- | | | | | | |
| | piperidin-1-yl}methyl)- | | | | | | |
| | 1-methyl-1H- | | | | | | |
| | imidazo[4,5-b]pyridine | | | | | | |
| 272 | 2-({4-[2-chloro-4- | 0.0241 | C20H20ClF3N4 | 408.13 | 408.9 | 2.3 | I |
| | (trifluoromethyl)phenyl]- | | | | | | |
| | piperidin-1-yl}methyl)- | | | | | | |
| | 1-methyl-1H- | | | | | | |
| | imidazo[4,5-b]pyridine | | | | | | |
| 273 | 2-({4-[2-chloro-4- | 0.085 | C20H20ClF3N4 | 408.13 | 408.9 | 2.2 | I |
| | (trifluoromethyl)phenyl]- | | | | | | |
| | piperidin-1-yl}methyl)- | | | | | | |
| | 1-methyl-1H- | | | | | | |
| | imidazo[4,5-c]pyridine | | | | | | |
| 274 | 2-({Cis-4-[2- | 0.0755 | C21H23F3N4O | 404.18 | 405.3 | 2 | I |
| | (difluoromethoxy)-4- | | | | | | |
| | fluorophenyl]-3- | | | | | | |
| | methylpiperidin-1- | | | | | | |
| | yl}methyl)-1-methyl-1H- | | | | | | |
| | imidazo[4,5-b]pyridine | | | | | | |
| 275 | 2-({Cis-4-[2- | 0.0965 | C21H23F3N4O | 404.18 | 405 | 1.5 | I |
| | (difluoromethoxy)-4- | | | | | | |
| | fluorophenyl]-3- | | | | | | |
| | methylpiperidin-1- | | | | | | |
| | yl}methyl)-1-methyl-1H- | | | | | | |
| | imidazo[4,5-c]pyridine | | | | | | |
| 276 | 1-methyl-2-({4-[3- | 0.0885 | C21H23F3N4 | 388.19 | 389 | 1.6 | I |
| | methyl-4- | | | | | | |
| | (trifluoromethyl)phenyl]- | | | | | | |
| | piperidin-1-yl}methyl)- | | | | | | |
| | 1H-imidazo[4,5- | | | | | | |
| | c]pyridine | | | | | | |
| 277 | 1-methyl-2-({4-[3- | 0.0605 | C21H23F3N4 | 388.19 | 389 | 2.4 | I |
| | methyl-4- | | | | | | |
| | (trifluoromethyl)phenyl]- | | | | | | |
| | piperidin-1-yl}methyl)- | | | | | | |
| | 1H-imidazo[4,5- | | | | | | |
| | b]pyridine | | | | | | |
| 278 | 2-({4-[3-methoxy-4- | 0.245 | C21H23F3N4O | 404.18 | 405 | 1.3 | I |
| | (trifluoromethyl)phenyl]- | | | | | | |
| | piperidin-1-yl}methyl)- | | | | | | |
| | 1-methyl-1H- | | | | | | |
| | imidazo[4,5-c]pyridine | | | | | | |
| 279 | 2-({4-[3-methoxy-4- | 0.289 | C21H23F3N4O | 404.18 | 405 | 1.9 | I |
| | (trifluoromethyl)phenyl]- | | | | | | |
| | piperidin-1-yl}methyl)- | | | | | | |
| | 1-methyl-1H- | | | | | | |
| | imidazo[4,5-b]pyridine | | | | | | |
| 280 | 2-({4-[2-methoxy-4- | 0.0339 | C21H23F3N4O2 | 420.18 | 421.1 | 1.6 | I |
| | (trifluoromethoxy)phenyl]- | | | | | | |
| | piperidin-1- | | | | | | |
| | yl}methyl)-1-methyl-1H- | | | | | | |
| | imidazo[4,5-c]pyridine | | | | | | |
| 281 | 2-({4-[2-methoxy-4- | 0.0997 | C21H23F3N4O2 | 420.18 | 421.1 | 1.8 | I |
| | (trifluoromethoxy)phenyl]- | | | | | | |
| | piperidin-1- | | | | | | |
| | yl}methyl)-1-methyl-1H- | | | | | | |
| | imidazo[4,5-b]pyridine | | | | | | |

TABLE 9
Examples with Data
The following specific compounds were prepared following the similar procedures to
the preparations and examples described above, using the appropriate intermediates and
reagents.

| | | | | | | | |
|---|---|---|---|---|---|---|---|
| 126 | 2-({4-[4- | 0.0626 | C22H23F3N4 | 400.19 | 401.1 | 2.16 | I |
| | (trifluoromethyl)phenyl]- | | | | | | |
| | piperidin-1-yl}methyl)- | | | | | | |
| | 5,6-dihydro-4H- | | | | | | |
| | imidazo[4,5,1-ij]-1,7- | | | | | | |
| | naphthyridine | | | | | | |
| 127 | 2-{[Cis-4-(2,4- | 0.0471 | C22H24F2N4 | 382.2 | 383.1 | | I |
| | difluorophenyl)-3- | | | | | | |
| | methylpiperidin-1- | | | | | | |
| | yl]methyl}-5,6-dihydro- | | | | | | |
| | 4H-imidazo[4,5,1-ij]- | | | | | | |
| | 1,7-naphthyridine | | | | | | |
| 128 | 2-{[Cis-4-(3-chloro-4- | 0.134 | C22H24ClFN4 | 398.17 | 399 | 2.16 | I |
| | fluorophenyl)-3- | | | | | | |
| | methylpiperidin-1- | | | | | | |
| | yl]methyl}-5,6-dihydro- | | | | | | |
| | 4H-imidazo[4,5,1-ij]- | | | | | | |
| | 1,7-naphthyridine | | | | | | |
| 129 | 2-{[Cis-4-(4-chloro-2- | 0.0125 | C22H24ClFN4 | 398.17 | 399 | 1.9 | I |
| | fluorophenyl)-3- | | | | | | |
| | methylpiperidin-1- | | | | | | |
| | yl]methyl}-5,6-dihydro- | | | | | | |
| | 4H-imidazo[4,5,1-ij]- | | | | | | |
| | 1,7-naphthyridine | | | | | | |
| 134 | 2-{[Cis-4-(4-fluoro-2- | 0.0302 | C23H27FN4O | 394.22 | 395.1 | 1.9 | I |
| | methoxyphenyl)-3- | | | | | | |
| | methylpiperidin-1- | | | | | | |
| | yl]methyl}-5,6-dihydro- | | | | | | |
| | 4H-imidazo[4,5,1-ij]- | | | | | | |
| | 1,7-naphthyridine | | | | | | |
| 135 | 2-({4-[2-methoxy-4- | <0.00543 | C23H25F3N4O | 430.2 | 431.1 | 2.42 | I |
| | (trifluoromethyl)phenyl]- | | | | | | |
| | piperidin-1-yl}methyl)- | | | | | | |
| | 5,6-dihydro-4H- | | | | | | |
| | imidazo[4,5,1-ij]-1,7- | | | | | | |
| | naphthyridine | | | | | | |

### O. Biological Protocols

In Vitro Assays

Procedure for mGluR2 Potentiator Screen NLB methods EC10-EC20 Challenge Cell Culture and Plating:

Cells used for this screen are HEK cells stably transfected with the mGluR2 receptor (metabotropic glutamate receptor 2) and the Gα15 G protein. Clones were identified by functional activity (FLIPR). Cells are grown in growth media containing: DMEM High Glucose with Glutamine and Na Pyruvate (GIBCO), 10% (v/v) Heat inactivate FBS (GIBCO), G418 500 ug/ml (from 50 mg/ml stock) (GIBCO) and Blasticidin 3 ug/ml (from 5 mg/ml stock made in H2O) (Invitrogen).

2 days before the assay cell are trypsinized with 0.25% trysin/EDTA (GIBCO), spun down at 1000 rpm for 5 minutes, resuspended in growth media and plated on polystyrene 384 well black wall 1 clear bottom poly-D-lysine coated plates at a density of approximately 18,000 cells/well in a volume of 50 μL per well. One day before the assay the growth media is removed from the plates by flicking, and replaced with media containing DMEM High Glucose without Glutamine and Na Pyruvate (GIBCO) and 10% (v/v) dialyzed FBS (GIBCO). The reason for the removal of glutamine the day before the assay is to minimize the amount of glutamate that will be present during the assay, as endogenous glutarnate released from the cells can reduce the fluorescent response and interfere with the FLIPR screen.

FLIPR Methods and Data Analysis:

On the day of the assay, the FLIPR assay is performed using the following methods.

Assay buffer:

| Compound | g/L | MW | [concentration] |
|---|---|---|---|
| NaCl | 8.47 | 58.44 | 145 mM |
| Glucose | 1.8 | 180.2 | 10 mM |
| KCl | .37 | 74.56 | 5 mM |
| MgSO₄ | 1 ml 1M Stock | 246.48 | 1 mM |
| HEPES | 2.38 | 238.3 | 10 mM |
| CaCl₂ | 2 ml 1M Stock | 110.99 | 2 mM |

The pH is adjusted to 7.4 with 1 M NaOH. Prepare a 2 mM (approx.) stock solution of Fluo-4,am (Molecular Probes) dye in DMSO-22 μl DMSO per 50 ug vial (440 μL per 1 mg vial). Make a 1 mM (approx.) flou-4, PA working solution per vial by adding 22 μl of 20% pluronic acid (PA), (Molecular Probes) in DMSO to each 50 ug vial (440 μL per 1 mg vial). Prepare a 250 mM Probenecid (Sigma) stock solution by dissolving 0.71 g into 5 ml 1N NaOH and 5 ml assay buffer (for each liter of assay wash buffer)L Make 4 uM (approx.) dye incubation media by adding 250 ug vials per 11 ml DMEM high glucose without glutamine (220 ml per 1 mg vial). Add 110 μL probenecid stock per 11 ml (2.5 mM final [concentration]). To the dye media add 3 units/ml of glutamic-pyruvic transaminase (GPT, Sigma) and 3 mM Na Pyruvate. The assay has worked with dye concentrations from 2 uM to 8 uM dye as well. To the assay buffer from drug preparation, add 1.83 mls DMSO and 400 μL 15.8% P104 (from New Leads biology) per liter for final concentrations of 0.18% DMSO and 0.006% P104. To the assay buffer for cell washing, add probenecid in the same manner and concentration that was used for the dye media.

Remove growth media from cell plates by flicking. Add 50 μl/well dye solution. Incubate 1 hour at 37° C. and 5% CO₂. Remove dye solution and wash 3 times with assay buffer+probenecid (100 μl probenecid stock per 10 ml buffer), leaving 30 μL/well assay buffer. Wait at least 10-15 minutes. Compounds and agonist challenge additions are performed with the FLIPR. The 1^{st} addition is for test compounds, which are added as 15 μL of 4×[concentration] of potentiator. The second 2^{nd} addition is 15 μL of 4×[concentration] of agonist or challenge. This achieves 1× concentration of all compounds only after 2^{nd} addition. The 1^{st} and 2^{nd} additions are performed separately using the FLIPR, which give 2 different data files. Compounds are pretreated at least 30 minutes before agonist addition,

Results are analyzed by dividing the peak fluorescent value of the FLIPR response by the time point after agonist addition to achieve a ratio response. The ratios are then analyzed by curve fitting programs. Since potent compounds can give an inverted U dose response curve (due to effects on endogenous glutamate by the potentiators), points are deleted at concentrations higher than the concentration that gives the maximum effect. Maximum values for dose response curves (forced fitting) are derived from standards on the plate.

Compound Preparation and Glutamate Challenge:

Compounds are delivered as 10 mM DMSO stocks or as powders, Powders are solubilized in DMSO at 10 mM (as solubility allows). Compounds are sonicated in a heated water bath (35-40° C.) for at least 20 minutes. Compounds are then added to assay drug buffer as 40 μL top [concentration] (4× the 10 uM top screening concentration).

In order to test compounds against an EC10 to EC20 concentration of glutamate, multiple glutamate challenge plates for the 2^{nd} FLIPR addition are prepared. The best challenge for a particular assay is determined by examining the glutamate dose response and 1-4 test plates.

EC₅₀ values of the compounds of the invention are preferably 10 micromolar or less more preferably 1 micromolar or less, even more preferably 100 nanomolar or less.

When introducing elements of the present invention or the exemplary embodiment(s) thereof, the articles “a,” “an,” “the” and “said” are intended to mean that there are one or more of the elements. The terms “comprising,” “including” and “having” are intended to be inclusive and mean that there may be additional elements other than the listed elements. Although this invention has been described with respect to specific embodiments, the details of these embodiments are not to be construed as limitations to the invention, the scope of which is defined by the appended claims.

1. A compound of formula I, or a pharmaceutically acceptable salt thereof, 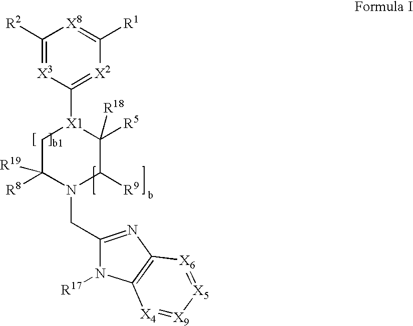
wherein:
X³═CR⁸
X²═CR⁴
X⁸═CR³
R¹, R², R³, R⁴ and R⁸ are each independently selected from the group consisting of hydrogen, halogen, —CN, —OR¹⁰¹, alkyl, alkenyl, cycloalkyl, cycloalkenyl, heterocycloalkylaryl, heteroaryl —C(O)OR¹⁰¹, —C(O)NR¹⁰¹R¹⁰², —NR¹⁰¹R¹⁰², and —NR¹⁰¹S(O)₂R¹⁰³ wherein each of R¹, R², R³, R⁴ and R⁶ alkyl, alkenyl, cycloalkyl, cycloalkenyl, heterocycloalkyl, aryl or heteroaryl is optionally independently substituted with one or more substituents independently selected from the group consisting of halogen, cyano, —R¹⁰¹, —OR¹⁰¹, —NR¹⁰¹R¹⁰², —S(O)_{q}R¹⁰³, —S(O)₂NR¹⁰¹R¹⁰², —NR¹⁰¹S(O)₂R¹⁰³, OC(O)R¹⁰³, —C(O)OR¹⁰³; —C(O)NR¹⁰¹R¹⁰², —NR¹⁰¹C(O)R¹⁰³, and C(O)R¹⁰³.
or two substituents bonded to adjacent carbon atoms of the ring containing X², X³ and X⁸, together with the adjacent carbon atoms, farm an heterocyclic or carbocyclic ring which is optionally substituted with—one or more R¹⁰, wherein each R¹⁰ is independently selected from the group consisting of hydrogen, cyano, halogen, —C(O)R¹⁰¹, —C(O)NR¹⁰¹R¹⁰², —NR¹⁰¹R¹⁰², —OR¹⁰¹, or —R¹⁰¹;
q is 0, 1 or 2;
each R¹⁰¹ and each R¹⁰² is independently selected from the group consisting of hydrogen, alkyl, alkenyl, alkynyl, cycloalkyl, aryl, heterocycloalkyl and heteroaryl;
wherein each R¹⁰¹ and R¹⁰² alkyl, alkenyl, alkynyl, cycloalkyl, aryl, heterocycloalkyl or heteroaryl is optionally independently substituted with one or more substituents independently selected from the group consisting of halogen, hydroxy, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl;
R¹⁰³ independently selected from the group consisting of alkyl, alkenyl, cycloalkyl, aryl, heterocycloalkyl and heteroaryl and is optionally substituted with one or more substituents independently selected from the group consisting of halogen, hydroxy, cyano, nitro, amino, alkylamino, dialkylamino, alkyl optionally substituted with one or more halogen or alkoxy or aryloxy aryl optionally substituted with one or more halogen or alkoxy or alkyl or trihaloalkyl, heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl;
X¹═CR⁷;
b=0, 1 or 2;
b1=1 or 2;
each of R⁵, R⁸ and R⁹ is independently selected from the group consisting of halogen, cyano, —R⁴⁰¹, —OR⁴⁰¹, —C(O)OR⁴⁰¹ and —NR⁴⁰¹R⁴⁰²;
R⁷ is hydrogen, halogen, hydroxyl, alkyl, alkoxy, cyano or alkyl-CO—;
or R⁵ and R⁷ taken together form a second bond;
R¹⁸ is hydrogen, halogen or alkyl;
R¹⁹ is H or —R⁸ and —R¹⁹ together may form ═O;
wherein R⁴⁰¹ and R⁴⁰² are independently selected from the group consisting of hydrogen, alkyl alkenyl, cycloalkyl, aryl, heterocycloalkyl and heteroaryl;
wherein each of the R⁴⁰¹ and R⁴⁰² alkyl, alkenyl, cycloalkyl, aryl, heterocycloalkyl and heteroaryl substituents is optionally independently substituted with one or more substituents independently selected from the group consisting of halogen, hydroxy, cyano, nitro, —R⁴¹¹, —C(O)R⁴¹³, —C(O)OR⁴¹³, —C(O)NR⁴¹¹R⁴¹², —OR⁴¹¹, —OC(O)R⁴¹³, —NR⁴¹¹R⁴¹², —NR⁴¹¹C(O)R⁴¹³, —NR⁴¹¹C(O)OR⁴¹³, —NR⁴¹¹S(O)₂R⁴¹³, —S(O)ₜR⁴¹³, —S(O)₂NR⁴¹¹R⁴¹²;
t is 0, 1 or 2;
R⁴¹¹ and R⁴¹² are independently selected from the group consisting of hydrogen, alkyl, cycloalkyl, aryl, heterocycloalkyl and heteroaryl;
R⁴¹³ is independently selected from the group consisting of alkyl, cycloalkyl, aryl, heterocycloalkyl and heteroaryl;
wherein the R⁴¹¹, R⁴¹² and R⁴¹³ alkyl, cycloalkyl, aryl, heterocycloalkyl and heteroaryl substituents are each optionally independently substituted with one or more substituents independently selected from the group consisting of halogen, hydroxy, cyano, nitro, alkyl, aryl, heterocycloalkyl, heteroaryl, haloalkyl, hydroxyalkyl, carboxy, alkoxy and alkoxycarbonyl;
or R⁴ and R⁵ together with the atoms connecting R⁴ and R⁵ form a 5-7-membered carbocyclic or heterocyclic ring optionally containing a heteroatom selected from O, N and S;
or if b−1 and b1=1, R⁵ and R⁹ together with the atoms connecting R⁵ and R⁹ form a 5-7-membered carbocyclic or heterocyclic ring containing up to two heteroatoms selected from O, N and S, wherein the carbocyclic or heterocyclic ring is optionally substituted with one or more substitutents selected from halogen, cyano, alkyl, cycloalkyl, heterocycloalkyl, aryl, heteroaryl or —C(O)R²⁰, wherein R²⁰ is alkyl, cycloalkyl, heterocycloalkyl, aryl or heteroaryl and R²⁰ is optionally substituted with one or more substituents independently selected from the group consisting of alkyl, alkoxy, aryloxy, cyano, —CO₂-alkyl, and —OC(O)alkyl;
or R⁴ and R⁷ together with the atoms connecting R⁴ and R⁷ form a 5-7-membered carbocyclic or heterocyclic ring, wherein if the ring formed by R⁴ and R⁷ together with the atoms connecting R⁴ and R⁷ is a heterocyclic ring, the heterocyclic ring formed by R⁴ and R⁷ together with the atoms connecting R⁴ and R⁷ contains a heteroatom selected from the group of O, N and S;
or R⁵ and R⁷ together with the atoms connecting R⁴ and R⁷ form a 3-7-membered carbocyclic or heterocyclic ring, wherein if the ring formed by R⁵ and R⁷ together with the atoms connecting R⁵ and R⁷ is a heterocyclic ring, the heterocyclic ring formed by R⁵ and R⁷ together with the atoms connecting R⁵ and R⁷ contains a heteroatom selected from the group of O, N and S;
wherein the carbocyclic or heterocyclic ring formed by R⁴ and R⁷ together with the atoms connecting R⁴ and R⁷, or by R⁶ and R⁷ together with the atoms connecting R⁶ and R⁷ is optionally substituted with one or more substitutents independently selected from halogen, cyano, alkyl, cycloalkyl, heterocycloalkyl, aryl, heteroaryl and C(O)R²⁰, wherein R²⁰ is alkyl, cycloalkyl, heterocycloalkyl, aryl or heteroaryl and R²⁰ is optionally substituted with one or more alkyl, alkoxy aryloxy, cyano, CO₂-alkyl, or OC(O)alkyl;
R¹⁷ is selected from the group consisting of alkyl, alkenyl, cycloalkyl, and cycloalkenyl, wherein the R¹⁷ alkyl, alkenyl, cycloalkyl, or cycloalkenyl is optionally substituted with one or more substituents independently selected from the group consisting of halogen, hydroxy, cyano, nitro, —R⁵⁰³, —OR⁵⁰³, —NR⁵⁰¹R⁵⁰², —S(O)ᵥR⁵⁰³, —S(O)₂NR⁵⁰¹R⁵⁰², —NR⁵⁰¹S(O)₂R⁵⁰³, —OC(O)R⁵⁰³, —C(O)OR⁵⁰³, —C(O)NR⁵⁰¹R⁵⁰², —NR⁵⁰³C(O)R⁵⁰³, and —C(O)R⁵⁰³;
u is 0, 1 or 2;
wherein each R⁵⁰¹ and each R⁵⁰² is independently selected from the group consisting of hydrogen, alkyl, alkenyl, cycloalkyl, cycloalkenyl, aryl, heterocycloalkyl and heteroaryl;
X⁴═N or CR¹¹;
X⁹═N or CR¹²;
X⁵═N or CR¹³;
X⁸═N or CR¹⁴;
wherein one or two of X⁴, X⁵, X⁸ and X⁹ are N;
R¹¹, R¹², R¹³ and R¹⁴ are each independently selected from the group consisting of halogen, cyano, —R⁶⁰¹, —(O)OR⁶⁰¹, C(O)NR⁶⁰¹R⁶⁰², —OR⁶⁰¹, —NR⁶⁰¹R⁶⁰², and —NR⁶⁰¹C(O)R⁶⁰²;
wherein each R⁶⁰¹ and each R⁶⁰² is independently selected from the group consisting of hydrogen, alkyl, alkenyl, cycloalkyl, cycloalkenyl, aryl, heterocycloalkyl and heteroaryl;
wherein the R⁶⁰¹ and R⁶⁰² alkyl, alkenyl, cycloalkyl, aryl, heterocycloalkyl and heteroaryl substituents are each independently optionally substituted with one or more substituents independently selected from the group consisting of halogen, hydroxy, cyano, nitro, —R⁶¹¹, —C(O)R⁶¹³—C(O)OR⁶¹³, —NR⁶¹¹R⁶¹², —OR⁶¹¹, —OR(O)R⁶¹³, —NR⁶¹¹R⁶¹², —NR⁶¹¹C(O)R⁶¹³, —NR⁶¹¹C(O)OR⁶¹³, —NR⁶¹¹S(O)₂R⁶¹³, —S(O)ᵤR⁶¹³, —S(O)₂NR⁶¹¹R⁶¹²;
u is 0, 1 or 2,
each R⁶¹¹ and each R⁶¹² is independently selected from the group consisting of hydrogen, alkyl, cycloalkyl, aryl, heterocycloalkyl and heteroaryl;
each R⁶¹³ is independently selected from the group consisting of alkyl, cycloalkyl, aryl, heterocycloalkyl and heteroaryl;
wherein the R⁶¹¹, R⁶¹² and R⁶¹³ alkyl, cycloalkyl, aryl, heterocycloalkyl and heteroaryl substituents are each independently optionally substituted with one or more substituents independently selected from the group consisting of halogen, hydroxy, cyano, nitro, alkyl, aryl, heterocycloalkyl, heteroaryl, haloalkyl, hydroxyalkyl, carboxy, alkoxy and alkoxycarbonyl.

2. The compound of claim 1, or a pharmaceutically acceptable salt thereof, wherein R¹⁷ is selected from the group consisting of alkyl and cycloalkyl; wherein the R¹⁷ alkyl and cycloalkyl substituents are optionally substituted with one or more substituents independently selected from the group consisting of halogen, cyano, —OR⁵⁰¹, and NR⁵⁰¹R⁵⁰².

3. The compound of claim 1, or a pharmaceutically acceptable salt thereof, wherein R⁷ is hydrogen, fluoro or alkyl.

4. The compound of claim 1, or a pharmaceutically acceptable salt thereof, wherein two of R¹¹, R¹², R¹³ and R¹⁴ are independently selected from the group consisting of hydrogen, halogen, cyano, alkyl, alkoxy, cycloalkyl, aryl, heterocycloalkyl and heteroaryl, wherein the two R¹¹, R¹², R¹³ or R¹⁴ alkyl, cycloalkyl, aryl, heterocycloalkyl and heteroaryl substituents are optionally independently substituted as in the compound of formula I.

5. The compound of claim 4, or a pharmaceutically acceptable salt thereof, wherein two of R¹¹, R¹², R¹³ and R¹⁴ are independently selected from the group consisting of hydrogen, cyano and halogen.

6. The compound of claim 1, or a pharmaceutically acceptable salt thereof, wherein three of R¹¹, R¹², R¹³ and R¹⁴ are independently selected from the group consisting of hydrogen, halogen, cyano, alkyl, alkoxy, cycloalkyl, aryl, heterocycloalkyl and heteroaryl, wherein the three R¹¹, R¹², R¹³ or R¹⁴ alkyl, cycloalkyl, aryl, heterocycloalkyl and heteroaryl substituents are optionally independently substituted as in the compound of formula I.

7. The compound of claim 6, or a pharmaceutically acceptable salt thereof, wherein three of R¹¹, R¹², R¹³ and R¹⁴ are independently selected from the group consisting of hydrogen, cyano and halogen.

8. The compound of claim 1, or a pharmaceutically acceptable salt thereof, wherein b=1 and b1=0.

9. The compound of claim 1, or a pharmaceutically acceptable salt thereof, wherein b=1 and b1=1.

10. The compound of claim 1, or a pharmaceutically acceptable salt thereof, wherein the compound of formula I has the formula II 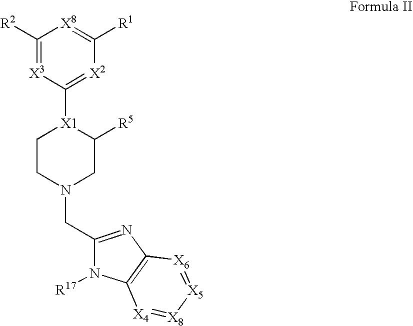
wherein,
R¹, R², R³, R⁴ and R⁶ are each independently selected from the group consisting of hydrogen, halogen, —CN, —OR¹⁰¹, alkyl, alkenyl, cycloalkyl, cycloalkenyl, heterocycloalkylaryl, heteroaryl, —C(O)R¹⁰¹, —C(O)OR¹⁰¹, —C(O)NR¹⁰¹R¹⁰², —NR¹⁰¹R¹⁰², and —NR¹⁰¹S(O)₂R¹⁰³ or, wherein each of R¹, R², R³, R⁴ and R⁶ alkyl, alkenyl, cycloalkyl, cycloalkenyl, heterocycloalkyl, aryl or heteroaryl is optionally independently substituted with one or more substituents independently selected from the group consisting of halogen, cyano, —R¹⁰¹, —OR¹⁰¹, —NR¹⁰¹R¹⁰², —S(O)_{q}R¹⁰³, —S(O)₂NR¹⁰¹R¹⁰², —NR¹⁰¹S(O)₂R¹⁰³, OC(O)R¹⁰³, —C(O)OR¹⁰³, —C(O)NR¹⁰¹R¹⁰², —NR¹⁰¹C(O)R¹⁰³, and —C(O)R¹⁰³;
R⁵ is selected from the group consisting of halogen, R⁴⁰¹, —OR⁴⁰¹, and —NR⁴⁰¹R⁴⁰²;
R⁷ is hydrogen, halogen, hydroxyl, alkyl, or alkoxy;
or R⁴ and R⁷ together with the atoms connecting R⁴ and R⁷ form a 5-7-membered carbocyclic or heterocyclic ring, wherein if the ring formed by R⁴ and R⁷ together with the atoms connecting R⁴ and R⁷ is a heterocyclic ring, the heterocyclic ring formed by R⁴ and R⁷ together with the atoms connecting R⁴ and R⁷ contains a heteroatom selected from the group of O, N and S;
or R⁵ and R⁷ together with the atoms connecting R⁵ and R⁷ form a 3-7-membered carbocyclic or heterocyclic ring, wherein if the ring formed by R⁵ and R⁷ together with the atoms connecting R⁵ and R⁷ is a heterocyclic ring, the heterocyclic ring formed by R⁵ and R⁷ together with the atoms connecting R⁵ and R⁷ contains a heteroatom selected from the group of O, N and S;
wherein the carbocyclic or heterocyclic ring formed by R⁴ and R⁷ together with the atoms connecting R⁴ and R⁷, or by R⁵ and R⁷ together with the atoms connecting R⁵ and R⁷, is optionally substituted with one or more substitutents independently selected from halogen, cyano, alkyl, cycloalkyl, heterocycloalkyl, aryl, heteroaryl and —C(O)R²⁰, wherein R²⁰ is alkyl, cycloalkyl, heterocycloalkyl, aryl or heteroaryl and R²⁰ is optionally substituted with one or more alkyl, alkoxy, aryloxy, cyano, —CO₂-alkyl, or —OC(O)alkyl.

11. The compound of claim 10, or a pharmaceutically acceptable salt thereof, wherein R⁷ is hydrogen or fluoro.

12. The compound of claim 10, or a pharmaceutically acceptable salt thereof, wherein R⁶ is hydrogen, halogen or alkyl optionally substituted with one or more fluorines.

13. The compound of claim 10, or a pharmaceutically acceptable salt thereof, wherein R¹⁷ is selected from the group consisting of alkyl and cycloalkyl, wherein the R alkyl and cycloalkyl substituent is optionally substituted as in the compound of formula II.

14. The compound of claim 10, or a pharmaceutically acceptable salt thereof, wherein two of X⁴, X⁵, X⁶ and X⁹ are N, and two of R¹¹, R¹², R¹³ and R¹⁴ are independently selected from the group consisting of hydrogen, halogen, cyano, alkyl, alkoxy, cycloalkyl, aryl, heterocycloalkyl and heteroaryl, wherein the two R¹¹, R¹², R¹³ or R¹⁴ alkyl, cycloalkyl, aryl, heterocycloalkyl and heteroaryl substituents are optionally independently substituted as in the compound of formula II.

15. The compound of claim 14, or a pharmaceutically acceptable salt thereof, wherein two of R¹¹, R¹², R¹³ and R¹⁴ are independently selected from the group consisting of hydrogen, cyano and halogen.

16. The compound of claim 10, or a pharmaceutically acceptable salt thereof, wherein one of X⁴, X⁵, X⁶ and X⁹ is N, and three of R¹¹, R¹², R¹³ and R¹⁴ are independently selected from the group consisting of hydrogen, halogen, cyano, alkyl, alkoxy, cycloalkyl, aryl, heterocycloalkyl and heteroaryl, wherein the three R¹¹, R¹², R¹³ or R¹⁴ alkyl, cycloalkyl, aryl, heterocycloalkyl and heteroaryl substituents are optionally independently substituted as in the compound of formula II.

17. The compound of claim 10, or a pharmaceutically acceptable salt thereof, wherein R⁵ and R⁷ together with the atoms connecting R⁵ and R⁷ form a 5-7-membered carbocyclic or heterocyclic ring, wherein the carbocyclic or heterocyclic ring is optionally substituted as in the compound of formula II.

18. The compound of claim 10, or a pharmaceutically acceptable salt thereof, wherein the compound of formula II has the formula III, 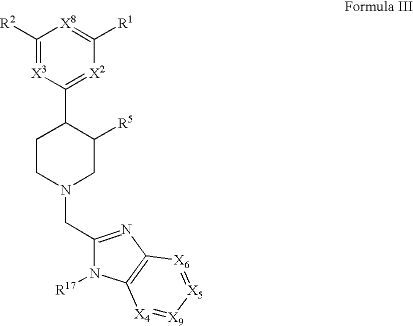
wherein
R¹, R², R³, R⁴ and R⁵ are each independently selected from the group consisting of hydrogen, halogen, —CN, —OR¹⁰¹, alkyl, alkenyl, cycloalkyl, cycloalkenyl, heterocycloalkylaryl, heteroaryl, —C(O)OR¹⁰¹, —C(O)NR¹⁰¹R¹⁰², —NR¹⁰¹R¹⁰², and —NR¹⁰¹S(O)₂R¹⁰³ or, wherein each of R¹, R², R³, R⁴ and R⁶ alkyl, alkenyl, cycloalkyl, cycloalkenyl, heterocycloalkyl, aryl or heteroaryl is optionally independently substituted with one or more substituents independently selected from the group consisting of halogen, cyano, R¹⁰¹, —OR¹⁰¹, —NR¹⁰¹R¹⁰², —S(O)_{q}R¹⁰³, —S(O)₂NR¹⁰¹R¹⁰², —NR¹⁰¹S(O)₂R¹⁰³, —OC(O)R¹⁰³, —C(O)OR¹⁰³, —C(O)NR¹⁰¹R¹⁰², —NR¹⁰³C(O)R¹⁰³, and —C(O)R¹⁰³;
and
R⁵ is hydrogen, halogen or alkyl optionally substituted with one or more fluorines

19. The compound of claim 18, or a pharmaceutically acceptable salt thereof, wherein one of X⁴, X⁵, X⁶ and X⁹ is N, and three of R¹¹, R¹², R¹³ or R¹⁴ are independently selected from the group consisting of hydrogen, halogen, cyano, alkyl, amino, heterocycloalkyl, aryl, and heteroaryl.

20. The compound of claim 18, or a pharmaceutically acceptable salt thereof, wherein one of X⁴, X⁵, X⁶ and X⁹ is N, and three of R¹¹, R¹², R¹³ and R¹⁴ are each independently selected from the group consisting of alkyl, cycloalkyl, heterocycloalkyl, heteroaryl and aryl each optionally independently substituted with one or more substituents independently selected from the group consisting of halogen, alkyl, haloalkyl, alkoxy and alkoxycarbonyl.

21. The compound of claim 18, or a pharmaceutically acceptable salt thereof, wherein two of X⁴, X⁵, X⁶ and X⁹ are N, and two of R¹¹, R¹², R¹³ and R¹⁴ are independently selected from the group consisting of hydrogen, halogen, cyano, alkyl, amino, heterocycloalkyl, aryl, and heteroaryl.

22. The compound of claim 18, or a pharmaceutically acceptable salt thereof, wherein two of X⁴, X⁵, X⁶ and X⁹ are N, and two of R¹¹, R¹², R¹³ and R¹⁴ are each independently selected from the group consisting of alkyl, cycloalkyl, heterocycloalkyl, heteroaryl and aryl each optionally independently substituted with one or more substituents independently selected from the group consisting of halogen, alkyl, haloalkyl, alkoxy and alkoxycarbonyl.

23. The compound of claim 18, or a pharmaceutically acceptable salt thereof, wherein R⁵ is hydrogen.

24. The compound of claim 18, or a pharmaceutically acceptable salt thereof, wherein R⁶ is alkyl.

25. The compound of claim 18, or a pharmaceutically acceptable salt thereof, wherein R⁶ and the aromatic ring containing X², X³ and X⁸ are cis- to each other.

26. The compound of claim 18, or a pharmaceutically acceptable salt thereof, wherein R¹⁷ is alkyl or cycloalkyl, wherein the R¹⁷ alkyl or cycloalkyl substituent is optionally substituted as in the compound of formula II.

27. The compound of claim 18, or a pharmaceutically acceptable salt thereof, wherein one of X⁴, X⁵, X⁶ and X⁹ is N, and three of R¹¹, R¹², R¹³ and R¹⁴ are independently selected from the group consisting of hydrogen, cyano, halogen, methyl, amino, methoxy, methoxypyridinyl and phenyl.

28. The compound of claim 18, or a pharmaceutically acceptable salt thereof, wherein two of X⁴, X⁵, X⁶ and X⁹ are N, and two of R¹¹, R¹², R¹³ and R¹⁴ are independently selected from the group consisting of hydrogen, cyano, halogen, methyl, amino, methoxy, methoxypyridinyl and phenyl.

29. The compound of claim 18, or a pharmaceutically acceptable salt thereof, wherein R¹⁷ is methyl, cyclopropyl, fluoroethyl, fluoromethyl, methoxyethyl or methoxymethyl.

30. The compound of claim 18, or a pharmaceutically acceptable salt thereof, wherein R¹⁷ is methyl, cyclopropyl, fluoroethyl, fluoromethyl, methoxyethyl or methoxymethyl;
and either
(a) one of X⁴, X⁵, X⁶ and X⁹ is N, and three of R¹¹, R¹², R¹³ and R¹⁴ are each hydrogen; or
(b) two of X⁴, X⁵, X⁶ and X⁹ are N, and two of R¹¹, R¹², R¹³ and R¹⁴ are each hydrogen.

31. The compound of claim 18, or a pharmaceutically acceptable salt thereof, wherein R¹⁷ is methyl; 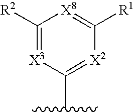
is phenyl optionally substituted with one or more substituents independently selected from the group consisting of halogen, cyano, alkyl, aryl, heterocycloalkyl, heteroaryl, haloalkyl, hydroxyalkyl, carboxy, alkoxy and alkoxycarbonyl; and
X⁶ is N.

32. The compound of claim 18, or a pharmaceutically acceptable salt thereof, wherein R¹⁷ is methyl: 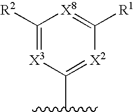
is phenyl optionally substituted with one or more substituents independently selected from the group consisting of halogen, cyano, alkyl, aryl, heterocycloalkyl, heteroaryl, haloalkyl, hydroxyalkyl, carboxy, alkoxy and alkoxycarbonyl; and
X⁵ is N.

33. The compound of claim 18, or a pharmaceutically acceptable salt thereof, wherein R¹⁷ is methyl; 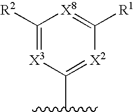
is phenyl optionally substituted with one or more substituents independently selected from the group consisting of halogen, cyano, alkyl, aryl, heterocycloalkyl, heteroaryl, haloalkyl, hydroxyalkyl, carboxy, alkoxy and alkoxycarbonyl; and
X⁴ is N.

34. The compound of claim 18, or a pharmaceutically acceptable salt thereof, wherein R¹⁷ is methyl; 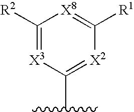
is phenyl optionally substituted with one or more substituents independently selected from the group consisting of halogen, cyano, alkyl, aryl, heterocycloalkyl, heteroaryl, haloalkyl, hydroxyalkyl, carboxy, alkoxy and alkoxycarbonyl; and
X⁹ is N.

35. The compound of claim 1, or a pharmaceutically acceptable salt thereof, wherein the compound of formula I has the formula IV, 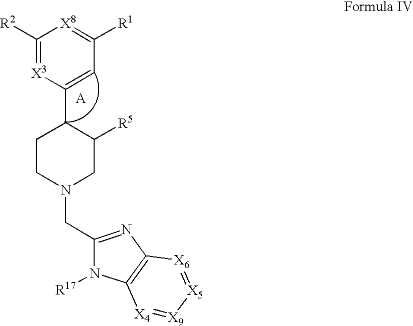
wherein,
X³═CR⁸
X⁸═CR³
R¹, R², R³, and R⁵ are each independently selected from the group consisting of hydrogen, halogen, —CN, —OR¹⁰¹, alkyl, alkenyl, cycloalkyl, cycloalkenyl, heterocycloalkylaryl, heteroaryl, C(O)R¹⁰¹, C(O)NR¹⁰¹R¹⁰², —NR¹⁰¹R¹⁰², or, wherein each of R¹, R², R³, and R⁶ alkyl, alkenyl, cycloalkyl, cycloalkenyl, heterocycloalkyl, aryl or heteroaryl is optionally independently substituted with one or more substituents independently selected from the group consisting of halogen, cyano, —R¹⁰¹, —OR¹⁰¹, —NR¹⁰¹R¹⁰³, —S(O)_{q}R¹⁰³, —S(O)₂NR¹⁰¹R¹⁰², —NR¹⁰¹S(O)₂R¹⁰³, —OC(O)R¹⁰³, —C(O)OR¹⁰³, —C(O)NR¹⁰¹R¹⁰², —NR¹⁰¹C(O)R¹⁰³, and —C(O)R¹⁰³;
R⁵ is hydrogen, halogen or alkyl;
and wherein ring A is a 5-7-membered carbocyclic or heterocyclic ring, wherein A is optionally substituted with one or more substitutents independently selected from halogen, cyano; alkyl optionally substituted with heterocycloalkyl; cycloakyl, heterocycloalkyl, aryl, heteroaryl —C(O)OR²⁰ or —C(O)R²⁰, wherein R²⁰ is alkyl, cycloalkyl, heterocycloalkyl, aryl or heteroaryl and R²⁰ is optionally substituted with one or more alkyl, alkoxy, aryloxy, cyano, —CO₂-alkyl, or —OC(O)alkyl.

36. The compound of claim 35, or a pharmaceutically acceptable salt thereof, wherein the compound of formula IV is a compound of formula IVa; 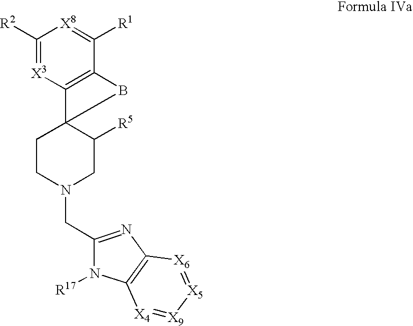
wherein B is a divalent chain selected from the group consisting of ethylene, ethynelene, propylene, butylene, methylenoxy, methylenethioxy, methylenamino, ethylenoxy, ethylenethioxy, and ethylenamino, wherein the carbons or the N of the methylenamino or ethylenamino divalent chain and the carbons of the ethylene, ethynelene, propylene, butylene, metheylenoxy, ethylenoxy, methylenethioxy, and ethylenethioxy divalent chain are each optionally independently substituted with one or more substitutents independently selected from halogen, cyano; alkyl optionally substituted with heterocycloalkyl, cycloalkyl, heterocycloalkyl, aryl, heteroaryl —CO(O)OR²⁰ or —C(O)R²⁰, wherein R²⁰ is alkyl, cycloalkyl, heterocycloalkyl, aryl or heteroaryl and R²⁰ is optionally substituted with one or more alkyl, alkoxy, aryloxy, cyano, —CO₂-alkyl, or —OC(O)alkyl.

37. The compound of claim 36, or a pharmaceutically acceptable salt thereof, wherein the N of the methylenamino or ethylenamino is optionally substituted with one or more substitutents independently selected from halogen, cyano, alkyl, cycloalkyl, heterocycloalkyl, aryl, heteroaryl or —C(O)R²⁰ wherein R²⁰ is alkyl, cycloalkyl, heterocycloalkyl, aryl or heteroaryl and R²⁰ is optionally substituted with one or more alkyl, alkoxy, aryloxy, cyano, —CO₂-alkyl, or —OC(O)alkyl.

38. The compound of claim 1, or a pharmaceutically acceptable salt thereof, wherein 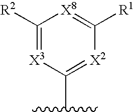
is selected from the group consisting of 4-fluoro-2-methoxyphenyl, 5-fluoro-2-methoxyphenyl, 5-chloro-2-methoxyphenyl, 5-chloro-2-ethoxyphenyl, 5-chloro-2-propoxyphenyl, 5-chloro-2-isobutoxyphenyl, isobutoxyphenyl, butoxyphenyl, 5-Chloro-2-((S)-2-methyl-butoxy)-phenyl, 5-Chloro-2-((R)-2-methyl-butoxy)-phenyl, 2-butoxy-5-chlorophenyl, 5-Chloro-2-(tetrahydro-pyran-2-ylmethoxy phenyl, 5-Chloro-2-(3-methyl-oxetan-3-ylmethoxy)-phenyl, 5-Chloro-2-(tetrahydro-furan-2-ylmethoxy)-phenyl, 5-Chloro-2-(tetrahydro-furan-3-ylmethoxy)-phenyl, 5-Chloro-2-(2-methyl-cyclopropylmethoxy)-phenyl, 5Chloro-2-(2-cyclopropyl-ethoxy)-phenyl, 5-Chloro-2-cyclobutylmethoxy-phenyl, cyclobutylmethoxy-phenyl, 4-fluoro-3-methoxyphenyl, 2-fluoro-6-methoxyphenyl, difluorophenyl, chlorofluorophenyl, chlorophenyl, bromophenyl, dibromophenyl, fluorophenyl, 2-methoxy-4-trifluoromethylphenyl, trifluoromethylphenyl, [dimethylmorpholin-4-yl]methylphenyl, (2-morpholin-4-yl-ethoxy)-phenyl, methylphenyl, dimethylphenyl, 4-chloro-3-trifluoromethylphenyl, methoxyphenyl, dimethoxyphenyl, hydroxyphenyl, phenyl, fluorophenyl, cyclopentylaminocarbonylphenyl, [N-cyclopropylmethyl]propylminocarbonylphenyl, [methylpyridynyl]aminocarbonylphenyl, fluorochromanyl, ethylphenyl, t-butylphenyl, cyanophenyl, trifluoromethoxyphenyl, isopropoxyphenyl, 2-methoxy-5-trifluoromethylphenyl, 2-fluoro-trifluoromethylphenyl, 2-fluoro-4-trifluoromethylphenyl, bis-trifluoromethylphenyl, hydroxyethylphenyl, 4-fluoro-2-methylphenyl, 5-Chloro-2-prop-2-ynyloxy-phenyl, prop-2-ynyloxy-phenyl, naphthalenyl, aminocarbonylnaphthalenyl, (1-phenyl-ethoxy)-phenyl, (indan-2-yloxy)-phenyl, ((S)-(tetrahydro-furan-3-yl)oxy]-phenyl, (tetrahydro-pyran-4-yloxy)-phenyl, ((S)-1-methyl-pyrrolidin-2-ylmethoxy)-phenyl, (2-pyridin-2-yl-ethoxy)-phenyl, ((S)-2-methyl-butoxy)-phenyl, cyclopropyl-ethoxyphenyl, pentoxyphenyl, 3-ethoxypropoxyphenyl, 2-ethoxyethoxyphenyl, 2 isopropoxyethoxyphenyl, 3-dimethylaminopropoxyphenyl, cyclopentylmethoxyphenyl, 2-(2,6-Dimethyl-morpholin-4-yl)-ethoxy]-phenyl, (2,6-Dimethyl-morpholin-4-yl)-phenyl, methoxycarbonylphenyl, methylsulfonyamidophenyl, methyl-cyclopropylmethoxyphenyl, propynyloxyphenyl, 5-chloro-2-propynyloxyphenyl, 5-chloro-2-(3-tetrahydrofuranyl)methoxyphenyl, 5-chloro-2-(3-tetrahydropyranyl)methoxyphenyl, 5-chloro-2-(2-tetrahydrofuranyl)methoxyphenyl, 5-chloro-2-(2-tetrahydropyranyl)methoxyphenyl, ethoxyphenyl, N-(5-methyl-1H-pyrazol-3-yl)aminocarbonylphenyl, 3-fluoro-4-trifluoromethyl-phenyl, 2-fluoro-4-trifluoromethoxyphenyl, 2-methyl-4-trifluoromethoxyphenyl, 4-chloro-4-methylphenyl, 4-fluoro-2-methylphenyl, 2-chloro-4-trifluoromethylphenyl, 2-chloro-4-isopropoxyphenyl, 2-fluoro-4-isopropoxyphenyl, 3-fluoro-4-isopropoxyphenyl, 3-chloro-4-isopropoxyphenyl, 3-chloro-4-ethoxyphenyl, 4-methoxy-2-trifluoromethylphenyl, difluoromethoxyphenyl, 2-fluoro-4-difluoromethoxyphenyl, 2-chloro-4-difluoromethoxyphenyl, trifluorophenyl, tetralinyl, 4-fluoro-2-isopropoxyphenyl, 4-fluoro-3-trifluoromethylphenyl, (2,3-dihydro-1-benzofuran-5-yl), 4-fluoro-2-trifluoromethylphenyl, 4-chloro-2-trifluoromethylphenyl, 2-chloro-4-methylphenyl, 3-chloro-4-trifluoromethoxyphenyl, 2-chloro-4-trifluoromethoxy-phenyl, 2-methoxy-4-trifluoromethoxyphenyl, 2-trifluoromethyl-4-isopropoxyphenyl, 2-fluoro-6-trifluoromethylphenyl, dichlorophenyl, 3-chloro-4-trifluoromethylphenyl, 2-methyl-4-trifluoromethylphenyl, 3-methyl-4-trifluoromethylphenyl, 4-fluoro-2-difluoromethoxyphenyl, 3 methoxy-4-trifluoromethylphenyl, and positional isomers thereof

39. The compound of claim 1, or a pharmaceutically acceptable salt thereof, wherein 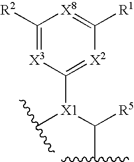
has the structure

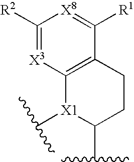

40. The compound of claim 1, or a pharmaceutically acceptable salt thereof, wherein R¹⁷ is selected from the group consisting of cycloalkyl, alkyl optionally substituted with halogen or with alkoxy.

41. The compound of claim 1, or a pharmaceutically acceptable salt thereof, wherein R⁴ and R⁵ together with the atoms connecting R⁴ and R⁵ form a 5-7-membered carbocyclic or heterocyclic ring optionally containing a heteroatom selected from O, N and S in which the carbocyclic or heterocyclic ring and the ring 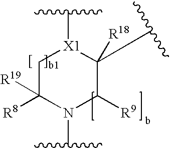
are cis-fused.

42. The compound of claim 1, or a pharmaceutically acceptable salt thereof, wherein R⁴ and R⁵ together with the atoms connecting R⁴ and R⁵ form a 5-7-membered carbocyclic or heterocyclic ring optionally containing a heteroatom selected from O, N and S in which the carbocyclic or heterocyclic ring and the ring 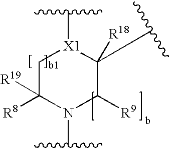
are trans-fused.

43. The compound of claim 1, or a pharmaceutically acceptable salt thereof, wherein the compound is an optically active compound of the formula 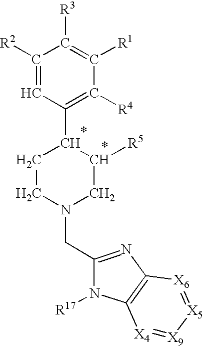
wherein R¹⁷ is as defined in formula I; three of X₆, X₅, X₉ and X₄ are CH and the fourth is N; R¹ and R² are each independently halogen or hydrogen; R³ is halogen, hydrogen, alkyl optionally substituted with halogen, or alkoxy optionally substituted with halogen; R⁴ is halogen, hydrogen, alkyl optionally substituted with halogen, or alkoxy; and R⁵ is alkyl optionally substituted with fluorine, wherein each of the carbons marked with an asterisk independently has the (R) configuration or the (S) configuration, provided that the R⁵ group and the phenyl group substituted with R¹, R², R³ and R⁴ are cis to each other.

44. The compound of claim 1, or a pharmaceutically acceptable salt thereof, wherein the compound is an optically active compound of the formula 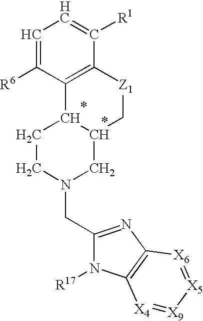
wherein R¹⁷ is as defined in formula I; three of X₆, X₅, X₉ and X₄ are CH and the fourth is N; Z₁ is O or CH₂, R¹ and R² are each independently halogen, hydrogen, or OR¹⁰¹ wherein R¹⁰¹ is alkyl or cycloalkyl, R³ is halogen, hydrogen, alkyl optionally substituted with halogen, or alkoxy optionally substituted with halogen; R⁶ is halogen or hydrogen, wherein each of the carbons marked with an asterisk independently has the (R) configuration or the (S) configuration.

45. A compound selected from the group consisting of the compounds disclosed in Table 8 herein or a pharmaceutically acceptable salt thereof.

46. A compound selected from the group consisting of the compounds disclosed in Table 7 herein or a pharmaceutically acceptable salt thereof.

47. A method for the treatment or prevention of a condition selected from the group consisting of cerebral deficits subsequent to cardiac bypass surgery and grafting, stroke, cerebral ischemia, spinal cord trauma, head trauma, perinatal hypoxia, cardiac arrest, hypoglycemic neuronal damage, dementia, Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine, urinary incontinence, substance tolerance, substance withdrawal, psychosis, schizophrenia, anxiety, mood disorders, trigeminal neuralgia, hearing loss, tinnitus, macular degeneration of the eye, emesis, brain edema, pain, tardive dyskinesia, sleep disorders, attention deficit/hyperactivity disorder, and conduct disorder in a mammal, comprising administering a compound of claim 1 or a pharmaceutically acceptable salt thereof to the mammal.

48. The method of claim 46, wherein the condition is anxiety selected from the group consisting of generalized anxiety disorder, social anxiety disorder, panic disorder, post-traumatic stress disorder and obsessive compulsive disorder.

49. The method of claim 46, wherein the condition is a mood disorder selected from the group consisting of depression, mania, and bipolar disorders.

50. A method for treating or preventing neurological and psychiatric disorders associated with glutamate dysfunction, comprising administering to a patient in need thereof an amount of a compound of claim 1, or a pharmaceutically acceptable salt thereof, effective in treating such disorders.

51. The method of claim 50, wherein further comprising administering a metabotropic glutamate receptor agonist.

52. A pharmaceutical composition comprising a compound of claim 1, or a pharmaceutically acceptable salt thereof, and a pharmaceutically acceptable carrier.

53. A composition for treating or preventing a condition selected from the group consisting of cerebral deficits subsequent to cardiac bypass surgery and grafting, stroke, cerebral ischemia, spinal cord trauma, head trauma, perinatal hypoxia, cardiac arrest, hypoglycemic neuronal damage, dementia, Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine, urinary incontinence, substance tolerance, substance withdrawal, psychosis, schizophrenia, anxiety, mood disorders, trigeminal neuralgia, hearing loss, tinnitus, macular degeneration of the eye, emesis, brain edema, pain, tardive dyskinesia, sleep disorders, attention deficit/hyperactivity disorder, and conduct disorder in a mammal, wherein the composition contains an amount of the compound of claim 1, or a pharmaceutically acceptable salt thereof, that is effective in the treatment or prevention of such conditions.

54. The composition of claim 52, further comprising a metabotropic glutamate receptor agonist.
